(19) United States
(12) Patent Application Publication
(10) Pub. No.: US 2021/0177795 A1
(43) Pub. Date: Jun. 17, 2021

(54) ADMINISTERING COMPOUNDS

(71) Applicant: Ena Therapeutics Pty Ltd, Melbourne, Victoria (AU)

(72) Inventors: David Jackson, Melbourne (AU); Francesca Mercuri, Melbourne (AU); Georgia Deliyannis, Melbourne (AU); Chinn Yi Wong, Melbourne (AU); Christophe Demaison, Melbourne (AU)

(21) Appl. No.: 16/768,278

(22) PCT Filed: Dec. 21, 2018
(86) PCT No.: PCT/AU2018/051401

§ 371 Date: May 29, 2020

(30) Foreign Application Priority Data
Dec. 21, 2017 (AU) 2017905126
Mar. 29, 2018 (AU) 2018901058

(51) Int. Cl. A61K 31/23; A61K 9/00; A61K 47/64; A61K 47/60; A61P 31/16

(52) U.S. Cl. CPC A61K 31/23; A61K 9/0043; A61P 31/16; A61K 47/60; A61K 47/6455

(57) ABSTRACT

The present invention relates to methods of treating or preventing respiratory conditions. In particular, the methods relate to treatment of respiratory conditions associated with a virus, such as influenza. In particular, the present invention provides a method of treating or preventing a respiratory condition associated with an infectious agent in an individual, the method comprising administering a compound comprising a TLR2 agonist to the upper respiratory tract of the individual, thereby treating or preventing a respiratory condition associated with an infectious agent in the individual. The compound is not administered to the lower respiratory tract or to both the upper and lower respiratory tract (i.e. administered to the total respiratory tract).

### CROSS-REFERENCE TO EARLIER APPLICATIONS

This application claims priority to Australian provisional applications AU 2017905126 and AU 2018901058, the entire contents of each are herein incorporated by reference in their entirety.

### FIELD OF THE INVENTION

The present invention relates to methods of treating or preventing respiratory conditions. In particular, the methods relate to treatment of respiratory conditions associated with a virus, such as influenza, or bacterium.

### BACKGROUND OF THE INVENTION

Respiratory infections are among the most common causes of human disease worldwide and are commonly caused by viruses.

Rhinoviruses (RV) are one of the most common types of virus to infect humans and are known to cause the common cold. The human rhinoviruses currently comprise the RV-A, RV-B, and RV-C species of the Enterovirus genus in the Picornaviridae family. Unlike sporadic pandemic and seasonal influenza outbreaks, rhinovirus infections occur throughout the year with multiple different serotypes. On average children experience 5-10 colds per year and well over half of all colds are caused by RV infection.

Viral respiratory infections can worsen the severity of diseases of the respiratory tract leading to exacerbations (attacks). Exacerbations can occur for conditions such as asthma and chronic obstructive pulmonary disease (COPD). Asthma and COPD exacerbations are the most clinically and economically important forms of the diseases. Rhinovirus is the most common viral infection associated with asthma exacerbations and therefore accounts for the greatest burden in terms of morbidity, mortality and health care cost.

Despite widespread vaccination initiatives, influenza remains a major cause of mortality and morbidity. Each year between 250,000 and 500,000 deaths are attributed to seasonal influenza, with associated annual healthcare costs in the US alone reaching billions of dollars.

Vaccination programmes have been developed and are aimed at minimising the burden of seasonal influenza, with the majority of vaccines designed to generate protective antibody-mediated immunity. However, the vaccines currently utilised are highly strain specific, especially in the case of killed virus vaccines.

Influenza viruses can evade established protective immune responses by two distinct mechanisms: either via the gradual antigenic drift of viral surface epitopes, or less commonly, through the emergence of new viral strains arising from re-assortment of influenza virus RNA from different strains in a common host.

There is therefore a need for new or improved therapies for the treatment and/or prevention of respiratory infections and/or respiratory conditions, particularly those associated with an infectious agent such as a virus or bacterium.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

The present invention provides a method of treating or preventing a respiratory condition associated with an infectious agent in an individual, the method comprising administering a compound comprising a Toll-like receptor 2 (TLR2) agonist to the upper respiratory tract of the individual, thereby treating or preventing a respiratory condition associated with an infectious agent in the individual.

In any embodiment, the method further comprises a step of identifying a subject having a respiratory condition associated with an infectious agent.

The present invention further provides for use of a compound comprising a TLR2 agonist in the preparation of a medicament for treating or preventing a respiratory condition associated with an infectious agent in an individual. Preferably, the medicament is adapted for administration to the upper respiratory tract.

In any embodiment, the invention also provides for use of a compound comprising a TLR2 agonist for the treatment or prevention of a respiratory condition associated with an infectious agent in an individual. Preferably, the compound comprising a TLR2 agonist is adapted for use in the upper respiratory tract.

In any aspect of the present invention, a compound comprising a TLR2 agonist is administered to the upper respiratory tract only. In other words, the compound comprising a TLR2 agonist is not administered to the lower respiratory tract or to both the upper and lower respiratory tract (i.e. administered to the total respiratory tract).

The present invention provides a method of inhibiting or reducing the amount of an infectious agent in the lung of an individual, the method comprising administering a compound comprising a TLR2 agonist to the upper respiratory tract of the individual, thereby inhibiting or reducing the amount of an infectious agent in the lung of an individual.

The present invention further provides for use of a compound comprising a TLR2 agonist in the preparation of a medicament for inhibiting or reducing the amount of an infectious agent in the lung of an individual. Preferably, the medicament is adapted for administration to the upper respiratory tract.

In any embodiment, the invention also provides for use of a compound comprising a TLR2 agonist for inhibiting or reducing the amount of an infectious agent in the lung of an individual. Preferably, the compound comprising a TLR2 agonist is adapted for use in the upper respiratory tract.

The present invention also provides a method of inhibiting, delaying or reducing the progression of an infectious agent from the upper respiratory tract to the lungs of an individual, the method comprising administering a compound comprising a TLR2 agonist to the upper respiratory tract of the individual, thereby inhibiting, delaying or reducing the progression of the infectious agent from the upper respiratory tract to the lungs of the individual.

The present invention further provides for use of a compound comprising a TLR2 agonist in the preparation of a medicament for inhibiting, delaying or reducing the progression of an infectious agent from the upper respiratory tract to the lungs of an individual. Preferably, the medicament is adapted for administration to the upper respiratory tract.

In any embodiment, the invention also provides for use of a compound comprising a TLR2 agonist for inhibiting, delaying or reducing the progression of an infectious agent from the upper respiratory tract to the lungs of an individual. Preferably, the compound comprising a TLR2 agonist is adapted for use in the upper respiratory tract.

Preferably, any method of inhibiting, delaying or reducing the amount or the progression of an infectious agent from the upper respiratory tract to the lungs of an individual reduces or prevents replication and dissemination of influenza virus from the upper respiratory tract to the lungs. Preferably, the TLR2 agonist is retained in the upper respiratory tract. In other words, the administration of the TLR2 agonist to the upper respiratory tract prevents or reduces viral dissemination into the lungs.

In any embodiment of the invention, there is no significant increase in levels of one or more pro-inflammatory cytokines as shown in the Examples in the lungs of an individual. Preferably, there is no significant increase of one or more of pro-inflammatory cytokines IL-10, IL-6, KC, MCP-1, RANTES, IL-12 or TNF-α in the lungs or lower respiratory tract when compared to pro-inflammatory cytokine levels of the upper respiratory tract.

As used herein, the upper respiratory tract may include any one or more of the following regions: the nose and nasal passages, paranasal sinuses, the pharynx, and the portion of the larynx above the vocal folds (cords). Typically, the lower respiratory tract includes any one or more of the following regions: the portion of the larynx below the vocal folds, trachea, bronchi and bronchioles. The lungs can be included in the lower respiratory tract and include the respiratory bronchioles, alveolar ducts, alveolar sacs, and alveoli. In any aspect of the present invention, administration to the URT may be administration to any one or more of the nose and nasal passages, paranasal sinuses, the pharynx, and the portion of the larynx above the vocal folds (cords).

Consequently, a compound or composition as described herein may be (a) administered to the nose and nasal passages, (b) administered to the nose, nasal passages and paranasal sinuses, (c) administered to the nose, nasal passages, paranasal sinuses and the pharynx, or (d) administered to the nose, nasal passages, paranasal sinuses, the pharynx and the portion of the larynx above the vocal folds (cords).

In any aspect of the present invention, the infectious agent is a virus or bacteria. Preferably, the infectious agent is a virus. Typically, the virus is influenza.

In any aspect of the present invention, the method or use comprises administering only a compound comprising a TLR2 agonist. In other words, the method does not comprise administering agonists of TLRs other than TLR2 homodimers or heterodimers.

The compound may be administered in a composition. Typically, the composition further comprises a pharmaceutically acceptable carrier, diluent or excipient. The composition may be formulated for administration to the upper respiratory tract, for example by intranasal administration. The composition may be free of compounds that are agonists of TLRs other than TLR2 homodimers or heterodimers. Preferably, the composition consists essentially of, or consists of a compound comprising a TLR2 agonist and a pharmaceutically acceptable carrier, diluent or excipient.

In any embodiment, the influenza infection for which prevention is required is an infection with a virus selected from the group consisting of influenza Types A, B or C. Influenza Type A virus can be subdivided into different serotypes or subtypes based on the antibody response to these viruses. Influenza A viruses are divided into subtypes based on two proteins on the surface of the virus: the hemagglutinin (H) and the neuraminidase (N). There are 18 different hemagglutinin subtypes and 11 different neuraminidase subtypes (H1 through H18 and N1 through N11 respectively). The sub types that have been confirmed in humans are H1N1, H1N2, H2N2, H3N2, H5N1, H7N2, H7N3, H7N7, H9N2 and H10N7.

In any aspect of the invention, the condition may be caused by a rhinovirus. Further, in any aspect of the invention, the viral mediated exacerbation is rhinovirus mediated. The rhinovirus may be any serotype as described herein. Typically, the rhinovirus is a member of the RV-A, RV-B, or RV-C rhinovirus species.

The present invention provides for methods for minimising the severity of a symptom associated with influenza infection, comprising administering a compound comprising a TLR2 agonist to the upper respiratory tract of the individual, wherein the symptoms are selected from the group consisting of chills, fever, sore throat, muscle pains, severe headache, coughing, weakness/fatigue and general discomfort.

In any embodiment of the present invention, the methods or uses are for treating or preventing a respiratory condition in an individual who is susceptible to an infection with a virus, preferably an influenza virus or rhinovirus.

In any aspect of the invention, the TLR2 agonist comprises a lipid, a peptidoglycan, a lipoprotein or a lipopolysaccharide. Preferably, the TLR2 agonist comprises palmitoyl, myristoyl, stearoyl, lauroyl, octanoyl, or decanoyl. The TLR2 agonist may be selected from the group consisting of: Pam2Cys, Pam3Cys, Ste2Cys, Lau2Cys, and Oct2Cys. In a preferred embodiment, the TLR2 agonist comprises Pam2Cys.

In any aspect of the invention, the compound comprises a soluble TLR2 agonist.

In any aspect of the invention, the TLR2 agonist may be conjugated with other compounds or functional groups. Other compounds or functional groups are any of those described herein. Preferred compounds are partially selected on the basis of their ability to assist in dissolving the TLR2 agonist in a carrier, diluent, excipient or solvent.

Depending on the polarity of the solvent, the solubility of the TLR2 agonist may be increased by a solubilising agent. Therefore, the compound may comprise a TLR2 agonist and a solubilising agent. Preferably, the TLR2 agonist and solubilising agent are linked. An exemplary solubilizing agent is polyethylene glycol (PEG) and in that embodiment the TLR2 agonist may be PEGylated. Preferably, the solubilising agent is any molecule as described herein.

The solubilising agent may comprise, consist essentially of, or consist of a positively or negatively charged group. Preferably, the charged group is a branched or linear peptide. Preferably, the positively charged group comprises at least one positively charged amino acid, such as an arginine (R) or lysine residue (K). Preferably, the negatively charged group comprises at least one negatively charged amino acid, such as glutamate (E) or aspartate (D). The charged amino acids may be terminal, preferably N-terminal.

Typically, the solubilising agent comprises polyethylene glycol (PEG), K4, H8 or R4. In any aspect of the invention, the solubilising agent comprises polyethylene glycol (PEG) and R4.

In any aspect of the invention, the compound comprises Pam2Cys conjugated to PEG₁₁. Preferably, the Pam2Cys and PEG₁₁ molecules are separated by one or two serines (S).

In any aspect of the invention, the compound comprising a TLR2 agonist is any one described herein, such that those described below.

In any aspect of the invention, the composition comprising, consisting essentially of or consisting of a compound comprising a TLR2 agonist is any one described herein, such that those described below.

In any aspect of the invention, the TLR2 agonist is not Pam3Cys.

In any aspect of the invention, the compound comprising a TLR2 agonist is INNA-002, INNA-003, Pam2CysSK4, INNA-006 or INNA-011 as described herein.

In any aspect of the invention, the compound comprising the TLR2 agonist may be administered in a single dose or multiple doses. The compound comprising a TLR2 agonist may be administered once or 3 times in a 24 hour period, or once or 3 times in a 7 day period. The frequency and timing of administration may be as described in the Examples.

In any aspect of the invention, the compound comprising the TLR2 agonist to be administered in a single dose or multiple doses is administered in an effective amount. An effective amount for a human subject lies in the range of about 250 nmoles/kg body weight/dose to 0.005 nmoles/kg body weight/dose. Preferably, the range is about 250 nmoles/kg body weight/dose to 0.05 nmoles/kg body weight/dose. In some embodiments, the body weight/dose range is about 250 nmoles/kg, to 0.1 nmoles/kg, about 50 nmoles/kg to 0.1 nmoles/kg, about 5 nmoles/kg to 0.1 nmol/kg, about 2.5 nmoles/kg to 0.25 nmoles/kg, or about 0.5 nmoles/kg to 0.1 nmoles/kg body weight/dose. In some embodiments, the amount is at, or about, 250 nmoles, 50 nmoles, 5 nmoles, 2.5 nmoles, 0.5 nmoles, 0.25 nmoles, 0.1 nmoles or 0.05 nmoles/kg body weight/dose of the compound.

In any aspect of the invention, compounds comprising a TLR2 agonist as described herein may be in compositions formulated for administration to the URT only. Preferably, compounds comprising a TLR2 agonist as described herein are formulated for intranasal administration. In a preferred embodiment, the composition is formulated as a nasal spray or as nasal drops.

In any aspect of the invention where prevention or prophylaxis is intended or required, the compound is administered to the subject before any clinically or biochemically detectable symptoms of viral infection, preferably influenza or rhinovirus infection.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1. Distribution of Evans Blue in the nasal turbinates (nose), trachea, lungs and stomach of mice inoculated intranasally. Mice were inoculated intranasally with a 10 μl or 50 μl solution of Evans Blue dye while anaesthetised using isoflurane. The animals were then killed and the nasal turbinates (nose), trachea, lungs and stomach removed and examined (A) visually and (B) spectrophotometrically for the for the presence of dye.

FIG. 2. Time course of change in body weight of C57131/6 mice challenged with 500 pfu Udorn virus while anaesthetised. Humane end point (80% original weight): The horizontal line at 80% represents the limit of weight loss i.e. 20% acceptable according to the AEC.

FIG. 3. Kinetics of viral growth in nasal turbinates of C57BL/6 mice following intranasal inoculation with 500 pfu Udorn virus.

FIG. 4. Kinetics of viral growth in lungs of C57131/6 mice following challenge with 500 pfu Udorn virus.

FIG. 5. Percentage change in body weight of mice receiving URT treatment with INNA-002. Groups of 5 C57BL/6 mice were treated intranasally with various doses of INNA-002 in 10 μl of saline. After 24 hours mice were challenged intranasally with 500 pfu of Udorn influenza virus in 10 μl of PBS under isoflurane anaesthesia. The horizontal line at 80% represents the limit of weight loss i.e. 20% acceptable according to the AEC.

FIG. 6. Prophylactic INNA-002 URT treatment prior to URT challenge with Udorn virus. Groups of 5 C57BL/6 mice were inoculated intranasally with varying doses of INNA-002 in 10 μl of saline under isoflurane anasthesia. After 24 hours mice were challenged intranasally with 500 pfu of Udorn influenza virus in 10 μl of PBS under isoflurane anaesthesia. Viral titers in the lungs were determined by plaque formation in MDCK cell monolayers 4 days after viral challenge.

FIG. 7. Weight changes in mice following treatment with various doses of INNA-003 and subsequently challenged with 500 pfu Udorn virus. Change in body weight of C57BL/6 mice (n=5/group) that were inoculated intranasally with varying doses of INNA-003 in 10 μl of saline under isoflurane anasthesia and 24 hours later were challenged intranasally with 500 pfu of Udorn influenza virus in 10 μl of PBS under isoflurane anaesthesia. The horizontal line at 80% represents the limit of weight loss i.e. 20% acceptable according to the AEC.

FIG. 8. Prophylactic INNA-003 URT treatment prior to URT challenge with Udorn influenza virus. Groups of 5 C57BL/6 mice were inoculated intranasally with varying doses of INNA-003 in 10 ul of saline under isoflurane anasthesia and 24 hours later mice were challenged intranasally with 500 pfu of Udorn influenza virus in 10 ul of PBS under isoflurane anaesthesia. Viral titers in the lungs were determined by plaque formation in MDCK cells at day 4 post-challenge.

FIG. 9. Percentage change in body weight of mice receiving URT treatment with the different TLR2 agonists. Groups of C57BL/6 mice (n=5) were inoculated intranasally with various doses of (A) INNA-003, (B) Pam2Cys-SK4 or (C) INNA-006 in 10 μl of saline while anaesthetised. After 24 hours, mice were anaesthetized and challenged intranasally with 500 pfu of Udorn virus in 10 μl of saline. Error bars depict s.d. and the horizontal line at 80% represents the limit of weight loss i.e. 20% acceptable according to the AEC.

FIG. 10. Prophylactic URT treatment with TLR2 agonists prior to URT challenge with Udorn virus. Groups of C57BL/6 mice (5 animals per group) were inoculated intranasally with different doses of (A) INNA-003, (B) Pam2Cys-SK4 or (C) INNA-006 in 10 μl of saline while anaesthetised. After 24 hours, mice were anaesthetized by isoflurane inhalation and challenged intranasally with 500 pfu of Udorn influenza virus in 10 μl of saline. Viral titers in the lungs were determined by plaque formation in MDCK cell monolayers 5 days after viral challenge. Error bars depict s.d. and statistical significance (***P=0.0002 & *P=0.0322) were obtained using a one-way ANOVA with Tukey comparing all columns within a test.

FIG. 11. Percentage change in body weight of mice receiving URT treatment with multiple doses of INNA-003 or INNA-006. Groups of 5 C57BL/6 mice were treated intranasally with either 3 doses of agonists on day 0, 2 and 4 or a single dose on day 4. Each dose was administered to anaesthetized mice and contained either 0.5 nmoles or 0.05 nmoles doses of INNA-003 or INNA-006 in 10 μl of saline. All mice were weighed daily. Error bars depict s.d.

FIG. 12a. Cytokine/Chemokine profiles in nasal turbinates, trachea, lungs and sera of mice receiving INNA-006 (0.5 nmole) by the URT route. Groups of 5 C57BL/6 mice were inoculated intranasally with either 1 dose or 3 doses of agonists over a 5-days period with 0.5 nmoles doses of INNA-006 in 10 ul of saline under isoflurane anesthesia. Mice were killed 24 hours after the last dose administered and cytokine/chemokine profiles in the (A) nasal turbinates, (B) trachea, (C) lungs, and (D) sera were determined by cytometric bead array. Error bars depict s.d. Statistical significance (***P=0.0002, **P=0.0021 & *P=0.0322) is denoted by asterisks and was obtained using a one-way ANOVA with Tukey's test comparing to saline control groups.

FIG. 12b. Cytokine/Chemokine profiles in nasal turbinates, trachea, lungs and sera of mice receiving of INNA-003 (0.5 nmole) by the URT route. Groups of 5 C57BL/6 mice were inoculated intranasally with either 1 dose or 3 doses of agonists over a 5-days period with 0.5 nmoles doses of INNA-003 in 10 ul of saline under isoflurane anesthesia. Mice were killed 24 hours after the last dose administered and cytokine/chemokine profiles in the (A) nasal turbinates, (B) trachea, (C) lungs, and (D) sera were determined by cytometric bead array. Error bars depict s.d. Statistical significance (***P=0.0002, **P=0.0021 & *P=0.0322) is denoted by asterisks and was obtained using a one-way ANOVA with Tukey's test comparing to saline control groups.

FIG. 13. Comparison of single and triple dose regimes of INNA-003 and INNA-006 on cytokine/chemokine profiles in nasal turbinate, trachea, lungs and sera. Groups of 5 C57BL/6 mice were inoculated intranasally with either 1 dose or 3 doses of INNA-003 (0.5 nmoles or 0.05 nmoles) or INNA-006 (0.5 nmoles or 0.05 nmoles) in 10 μl of saline while anaesthetised with isoflurane. Mice were killed 24 hours after the last dose of TLR2 agonist and the level of cytokines in the nasal turbinate, trachea, lungs, and sera determined by cytometric bead array. Error bars indicate the s.d. and statistical significance (***P=0.0002, **P=0.0021 & *P=0.0322) is denoted by asterisks obtained using a oneway ANOVA with Tukey's test which was obtained by comparison with saline control groups.

FIG. 14. Percentage change in body weight of mice following multiple treatments with INNA-003 or INNA-006 followed by challenge with Udorn influenza virus. Groups of 5 C57BL/6 mice were inoculated intranasally with 3 doses of agonist over a 5-day period, each dose contained 0.5 nmole doses of INNA-003 or INNA-006 in 10 μl of saline and was administered to anaesthetized mice. Twenty four hours after the last dose, mice were challenged intranasally with 500 pfu of Udorn influenza virus in 10 μl of PBS while anaesthetized. Error bars depict s.d. and the horizontal line at 80% represents the limit of weight loss i.e. 20% acceptable according to the AEC.

FIG. 15. Effects on viral titre in the lungs of mice treated prophylactically with multiple doses of INNA-003 or INNA-006. Groups of 5 C57BL/6 mice were inoculated intranasally with 3 doses of agonist over a 5-day period, each dose contained 0.5 nmoles doses of INNA-003 or INNA-006 in 10 μl of saline and was administered to anaesthetized mice. Twenty four hours after the last dose, mice were challenged intranasally with 500 pfu of Udorn influenza virus in 10 μl of PBS while anaesthetized. Viral titers in the lungs were determined by plaque formation in MDCK cells at day 5 post-challenge. Error bars depict s.d. and statistical significance (**P=0.0021) is denoted by asterisks and was obtained using a one-way ANOVA with Tukey comparing all column test.

FIG. 16: Viral titres in lungs of mice following prophylactic treatment with INNA-011 to the URT prior to challenge with Udorn virus. Groups of 10 C57BL/6 mice were treated intranasally to the URT with 5 nmoles of INNA-011 in 10 μl of saline or with saline alone 7 days before challenge with Udorn virus. Mice were challenged intranasally with 500 pfu of Udorn influenza virus in 10 μl of PBS under isoflurane anaesthesia. Viral titres in the lungs were determined by plaque formation in MDCK cell monolayers 5 days after viral challenge. Error bars depict s.d. Statistical significance (**P=0.0021) is denoted by asterisks and was obtained using a one-way ANOVA with Tukey's test comparing all groups.

FIG. 17: Viral titres in lungs of mice following one day prophylactic treatment with INNA-011 to the URT prior to challenge with Udorn virus. (A) Groups of 5 C57BL/6 mice were treated intranasally with INNA-011 in 10 μl of saline on 1 day before challenge with Udorn virus. Mice were challenged intranasally with 500 pfu of Udorn virus in 10 μl of saline under isoflurane anaesthesia. Viral titers lungs were determined by plaque formation in MDCK cell monolayers 5 days after viral challenge. Error bars depict standard deviation. Statistical significance (***P=0.0002) is denoted by asterisks and was obtained using a one-way ANOVA with Dunnett test comparing all groups to saline group. (B) Groups of 7 C57BL/6 mice were treated intranasally with 1 nmole of INNA-011 in 10 μl of saline 1 day before challenge with Udorn virus. Mice were challenged intranasally with 500 pfu of Udorn virus in 10 μl of saline under isoflurane anaesthesia. Viral titers in the lungs were determined by plaque formation in MDCK cell monolayers 5 days after viral challenge. Error bars depict standard deviation. Statistical significance (*P=0.0332) is denoted by asterisks and was obtained using a one-way ANOVA with Dunnett test comparing to saline group. (C) Groups of 10 C57BL/6 mice were treated intranasally with 0.25 nmole of INNA-011 in 10 μl of PBS 1 day before challenge with Udorn virus. Mice were challenged intranasally with 500 pfu of Udorn virus in 10 μl of PBS under isoflurane anaesthesia. Viral titers in the lungs were determined by plaque formation in MDCK cell monolayers 5 days after viral challenge. Error bars depict s.d. Statistical significance (*P=0.0021) is denoted by asterisks and was obtained using a one-way ANOVA with Dunnett test comparing to saline group.

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

One skilled in the art will recognize many methods and materials similar or equivalent to those described herein, which could be used in the practice of the present invention. The present invention is in no way limited to the methods and materials described.

All of the patents and publications referred to herein are incorporated by reference in their entirety.

For purposes of interpreting this specification, terms used in the singular will also include the plural and vice versa.

Natural exposure to influenza results in virus-containing airborne droplets being deposited in the URT where viral replication occurs with subsequent dissemination to the lower respiratory tract. Very little, if any, virus is transported to the lungs on first contact with virus. A procedure for mimicking this natural process has been successfully established by the inventors. Specifically, the inventors have established an upper respiratory tract (URT) influenza virus challenge model in mice using a dose of infectious virus which replicates in the URT and then progress to the lungs. The URT model has been used to determine that, unexpectedly, localised administration of a compound comprising a TLR2 agonist to the URT can reduce or prevent replication and dissemination of influenza virus from the URT to the lungs. Localised administration to the URT in this context means that minimal to no compound contacts the lower respiratory tract (LRT), i.e. all compound that is administered is retained in the URT. At the very least, the contact with the LRT is such that there is no significant increase in one or more pro-inflammatory cytokines as shown in the Examples (i.e. in the LRT (such as the trachea and/or lung), and/or serum).

This surprisingly shows that administration of a compound comprising a TLR2 agonist to the URT, i.e. not to, or not significantly to, the lower respiratory tract or the total respiratory tract (TRT), is sufficient to inhibit viral dissemination into the lungs. An advantage of an aspect of the invention is that administration to the URT only allows less compounds to be used compared to administration to the TRT or LRT. Further, an advantage of an aspect of the invention is that a reduction in the dissemination of infectious agent from the URT to the lungs reduces the likelihood of significant infection requiring more aggressive treatments. Delivery of agents to the lungs can elicit harmful inflammatory responses and this is avoided or minimised with URT administration in accordance with aspects of the invention. This is particularly relevant where subjects in need of the treatment or prevention described herein may have pre-existing inflammation of the lung.

Lungs are sensitive and essential organs which are vulnerable to attack by infectious and other agents. Inhibiting/reducing viral dissemination to the lungs reduces damage to these sensitive organs by minimising damaging inflammatory responses and limiting pathogen-mediated apoptosis and necrosis.

Infectious Agents/Conditions

Influenza (commonly referred to as “the flu”) is an infectious disease caused by RNA viruses of the family Orthomyxoviridae (the influenza viruses) that affects birds and mammals. The most common symptoms of the disease are chills, fever, sore throat, muscle pains, severe headache, coughing, weakness/fatigue and general discomfort.

The influenza viruses make up three of the five genera of the family Orthomyxoviridae. Influenza Type A and Type B viruses co-circulate during seasonal epidemics and can cause severe influenza infection. Influenza Type C virus infection is less common but can be severe and cause local epidemics.

Influenza Type A virus can be subdivided into different serotypes or subtypes based on the antibody response to these viruses. Influenza A viruses are divided into subtypes based on two proteins on the surface of the virus: the hemagglutinin (H) and the neuraminidase (N). There are 18 different hemagglutinin subtypes and 11 different neuraminidase subtypes. (H1 through H18 and N1 through N11 respectively.) The sub types that have been confirmed in humans are H1N1, H1N2, H2N2, H3N2, H5N1, H7N2, H7N3, H7N7, H9N2 and H10N7.

Influenza has an enormous impact on public health with severe economic implications in addition to the devastating health problems, including morbidity and even mortality. Accordingly, there is a need for therapeutic agents which can prevent infection, or reduce severity of infection in individuals.

In any embodiment, the influenza infection for which prevention is required is an infection with a virus selected from the group consisting of influenza Types A, B or C. Influenza Type A virus can be subdivided into different serotypes or subtypes based on the antibody response to these viruses.

Influenza A viruses are divided into subtypes based on two proteins on the surface of the virus: the hemagglutinin (H) and the neuraminidase (N). There are 18 different hemagglutinin subtypes and 11 different neuraminidase subtypes (H1 through H18 and N1 through N11 respectively). The sub types that have been confirmed in humans are H1N1, H1N2, H2N2, H3N2, H5N1, H7N2, H7N3, H7N7, H9N2 and H10N7.

In any aspect of the invention, the condition may be caused by a rhinovirus. Further, in any aspect of the invention, the viral mediated exacerbation is rhinovirus mediated. The rhinovirus may be any serotype as described herein. Typically, the rhinovirus is a member of the RV-A, RV-B, or RV-C rhinovirus species.

Compounds and Compositions

Compounds that comprise a TLR2 agonist and compositions of such compounds that are useful in any aspect of the present invention are described below.

Toll-Like Receptors (TLRs) are pattern recognition receptors (PRRs) expressed by diverse cell types that play an important role in both innate and adaptive immunity. Cells of the innate immune system respond to TLR activation by producing pro-inflammatory cytokines and chemokines that signal for the clearance of the pathogens and damaged-self. Upon engagement with specific ligands, TLR activation leads to the activation of transcription factors such as nuclear factor kappa B (NF)-kB, activating protein-1 (AP-1) and interferon regulatory factors (IRFs) through several adaptor molecules including myeloid differentiation primary response gene 88 (MyD88), Toll-interleukin 1 receptor (TIR) domain containing adaptor protein TIRAP and TIR-domain containing adaptor inducing interferon-beta TRIF, to regulate cytokine expression.

There are a number of TLRs that belong to this membrane receptor protein family including TLR1, TLR2, TLR3, TLR4, TLR5, TLR6, TLR7, TLR8 and TLR9.

As used herein, the term “TLR2” is intended to mean Toll-Like Receptor 2 protein. In humans, TLR2 is encoded by the TLR2 gene. TLR2 is expressed on the surface of certain cells and plays a fundamental role in pathogen recognition and activation of innate immunity.

A TLR2 agonist is an agent that binds Toll-like receptor 2. The TLR2 agonist may bind to, and activate, TLR2 as a homodimer or heterodimer.

In any embodiment of the invention, the TLR2 agonist comprises a lipid, a peptidoglycan, a lipoprotein, a lipopeptide, or a lipopolysaccharide. Preferably, the TLR2 agonist comprises palmitoyl, myristoyl, stearoyl, lauroyl, octanoyl, or decanoyl. The TLR2 agonist may be selected from the group consisting of: Pam2Cys, Pam3Cys, Ste2Cys, Lau2Cys, and Oct2Cys. In a preferred embodiment, the TLR2 agonist comprises Pam2Cys.

An exemplary lipopeptide in accordance with any embodiment of the present invention is the lipopeptide “Pam2Cys”. One of skill in the art would understand that the term “lipopeptide” means any composition of matter comprising one or more lipid moieties and one or more amino acid sequences that are conjugated. “Pam2Cys” (also known as dipalmitoyl-S-glyceryl-cysteine or S-[2,3 bis(palmitoyloxy) propyl] cysteine has been synthesised and corresponds to the lipid moiety of MALP-2, a macrophage-activating lipopeptide isolated from Mycoplasma fermentans. Pam2Cys is known to be a ligand of TLR2.

Pam2Cys has the structure:

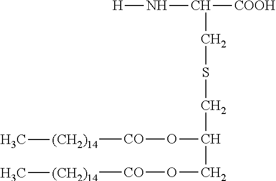

Another exemplary lipopeptide is the lipoamino acid N-palmitoyl-S-[2,3-bis (palmitoyloxy) propyl] cysteine, also known as Pam3Cys or Pam3Cys-OH and is a synthetic version of the N-terminal moiety of Braun's lipoprotein that spans the inner and outer membranes of Gram negative bacteria. Pam3Cys has the following structure:

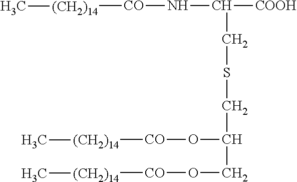

U.S. Pat. No. 5,700,910 describes several N-acyl-S-(2-hydroxyalkyl) cysteines for use as intermediates in the preparation of lipopeptides that are used as synthetic adjuvants, B lymphocyte stimulants, macrophage stimulants, or synthetic vaccines. U.S. Pat. No. 5,700,910 also teaches the use of such compounds as intermediates in the synthesis of Pam3Cys-OH and of lipopeptides that comprise this lipoamino acid or an analog thereof at the N-terminus.

Other lipid moieites which may be used to target cell surface TLRs include palmitoyl, myristoyl, stearoyl, lauroyl, octanoyl, or decanoyl.

In addition to Pam2Cys and Pam3Cys, the present invention also contemplates the use of Ste2Cys, Lau2Cys and Oct2Cys according to the present invention. Those skilled in the art will be aware that Ste2Cys is also known as S-[2,3-bis (stearoyloxy) propyl] cysteine or distearoyl-S-glyceryl-cysteine; that Lau2Cys is also known as S-[2,3-bis (lauroyloxy) propyl] cysteine or dilauroyl-S-glyceryl-cysteine); and that Oct2Cys is also known as S-[2,3-bis (octanoyloxy) propyl] cysteine or dioctanoyl-S-glyceryl-cysteine).

Other suitable TLR2 agonists include, but are not limited to, synthetic triacylated and diacylated lipopeptides, FSL-1 (a synthetic lipoprotein derived from Mycoplasma salivarium 1), Pam3Cys (tripalmitoyl-S-glyceryl cysteine) and S-[2,3-bis(palmitoyloxy)-(2 RS)-propyl]-N-palmitoyl-(R)-cysteine, where “Pam3” is “tripalmitoyl-S-glyceryl”. Derivatives of PanuCys are also suitable TLR2 agonists, where derivatives include, but are not limited to: S-[2,3-bis(palmitoyloxy)-(2-R,S)-propyl]-N-palmitoyl-(R)-Cys-(S)-Ser-(Lys)4-hydroxytrihydrochloride; Pam3Cys-Ser-Ser-Asn-Ala; Pam3Cys-Ser-(Lys)4; Pam3Cys-Ala-Gly; Pam3Cys-Ser-Gly; Pam3Cys-Ser; Pam3CyS-OMe; Pam3Cys-OH; PamCAG, palmitoyl-Cys((RS)-2,3-di(palmitoyloxy)-propyl)-Ala-Gly-OH, and the like.

Other non-limiting examples of suitable TLR2 agonists are Pam2CSK4 Pam2CSK4 (dipalmitoyl-S-glyceryl cysteine-serine-(lysine)4; or Pam2Cys-Ser-(Lys)4) is a synthetic diacylated lipopeptide. Other synthetic TLRs agonists include those described, e.g., in Kellner et al. (1992) Biol. Chem. 373:1:51-5; Seifer et al. (1990) Biochem. J, 26:795-802; and Lee et al. (2003) J. Lipid Res., 44:479-486.

A TLR2 agonist may be conjugated with one or more compounds or functional groups. Examples of particular compounds or functional groups are given below. One form of compound or functional group may act to increase the solubility of the TLR2 agonist. As will be understood by persons skilled in the art, TLR2 agonists are typically non-polar and, accordingly, while being soluble in non-polar solvents, are less soluble in polar and aqueous solvents. Where it is desired to use the TLR2 agonist in a polar or aqueous solvent, the TLR2 agonist may be conjugated with a solubilising agent.

A solubilising agent may include one, or more than one, solubilising agent which may be conjugated to TLR2 agonist in order to improve the solubility of the TLR2 moiety. The solubilising agent will generally be a polar moiety which increases the solubility of the TLR2 moiety in polar or aqueous solvents.

In any aspect of the invention, the solubilising agent may be a positively charged group. Positively charged groups of the present invention include but are not limited to penetratin, HIV Tat 48-60, HIV Rev 34-50, transportan, oligoarginine peptides (linear and branched), oligolysine peptides, pyrrrochoricin, alpha-helical amphipathic model peptide, polylysine, protamine, FL17, Magnafloc 1697, and the polycationic compounds described in U.S. Pat. Nos. 6,689,478 and 4,035,558.

In yet a further embodiment of the present invention, the solubilising agent comprises, consists essentially of, or consists of a linear or branched peptide. Typically, the linear or branched peptide contains positively or negatively charged amino acids. Positively charged amino acids may be lysine, arginine, histidine, ornithine or combinations thereof. The branched or linear peptide may contain at least one lysine or arginine residue. Preferably, the charged amino acids are terminal, for example N-terminal. The branched peptides may have one of the following structures.

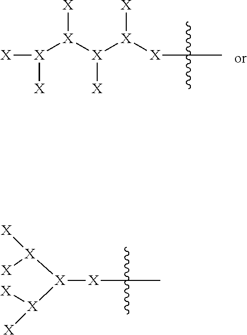

In the above structures X may independently be a charged residue, either a positively or negatively charged residue. Preferably the positively charged amino acids are lysine, arginine, histidine or ornithine. Preferably, the negatively charged amino acids are glutamate or aspartate.

The compound or functional group which can act as a solubilising agent may be one or more of the group consisting of “PEG” (or polyethyleneglycol) and a polar polypeptide such as “R4”, a hyper-branched tetra arginine complex; “H4”, a hyper-branched tetra histidine complex; “H8”, a linear peptide containing histidine residues; and “E8” a linear peptide containing glutamate residues. Other linear and branched lipid solubilising agents are also envisaged, including a hyper-branched peptide containing glutamate residues (see, e.g., “branched E8”, below). In yet a further embodiment of the present invention, the solubilising agent includes PEG and one or more of the group consisting of R4, H4, H8 and E8 (linear or branched). R4, H4, H8 and E8 have been previously described in PCT/AU2009/000469 (WO/2010/115230) and have the following structures:

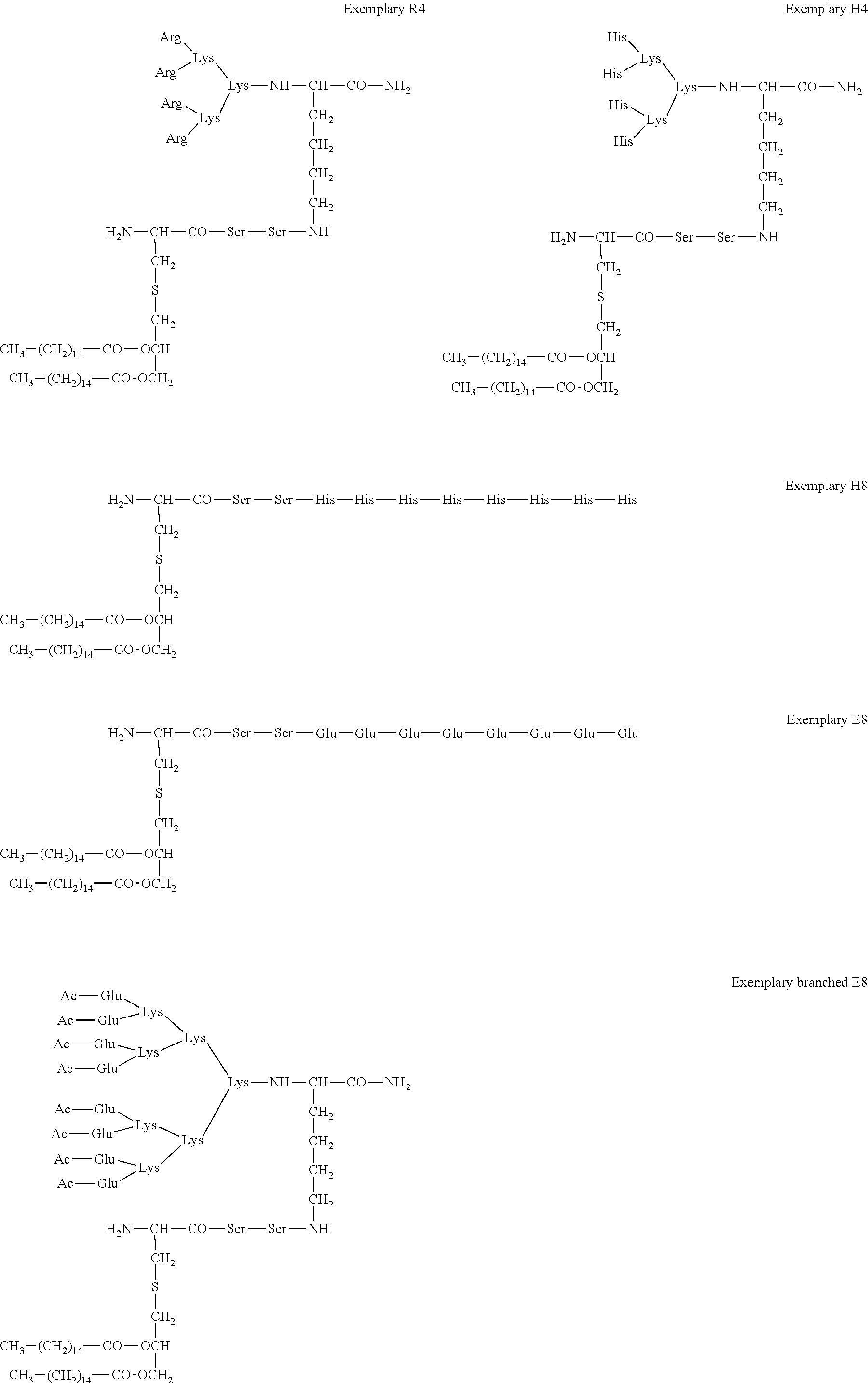

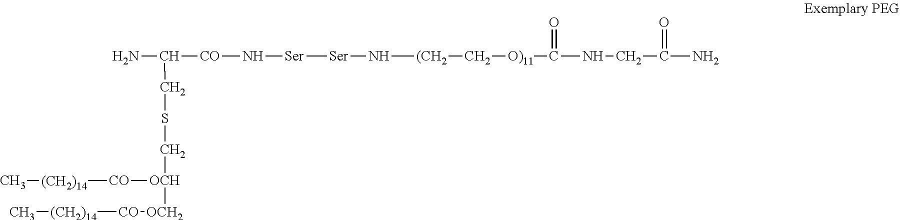

Following are schematic representations of some examples of branched (structures 1-5) and linear (structures 6-8) immunogenic compositions comprising of positively charged (Arginine, R; Lysine, K) or negatively charged (Aspartic acid, D; Glutamic acid, E) amino acids in terminal positions such that their respective electrostatic charges are displayed to the environment. Each immunogenic composition also contains dipalmitoyl-S-glyceryl cysteine (Pam2Cys) which is a ligand for Toll-Like Receptor 2. Two serine residues (Ser) are also incorporated. In the case of construct 2 the peptide structure was assembled in the direction N→C, all other structures shown in the figure were assembled C→N. Positive and negative electrostatic charges are shown as 2−, 2+, 1−, 1+ etc. depending on the size of charge. Ac=acetyl group used to suppress the positive charge of alpha amino groups in the case of N-terminally situated Glutamic acid.

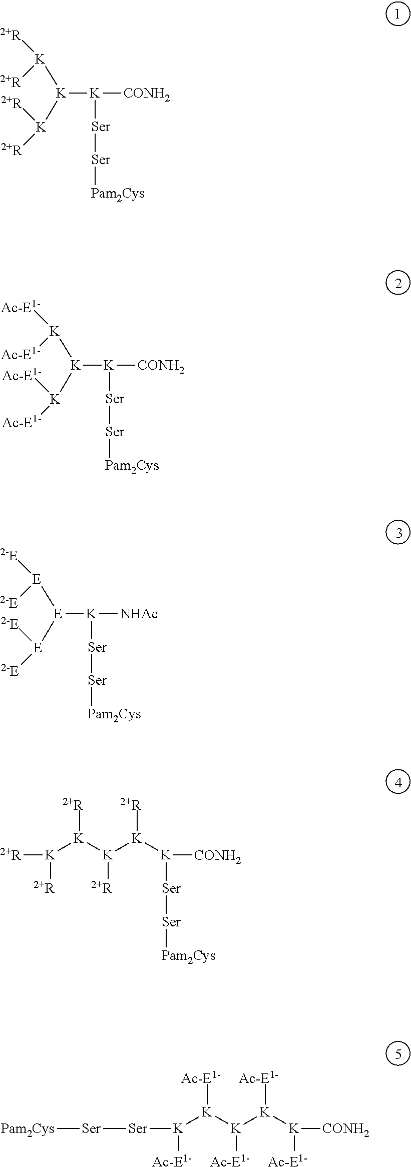

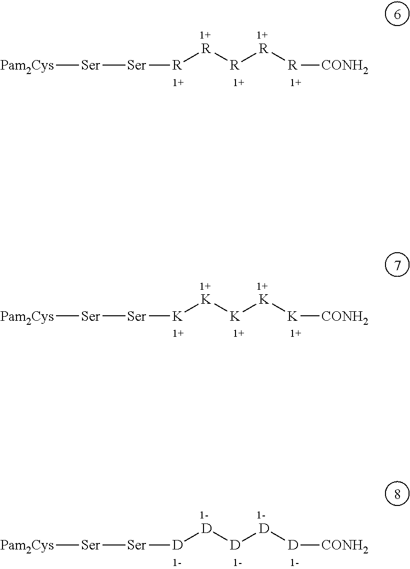

A person skilled in the art will appreciate that the present invention is not limited to the particular exemplified compounds or functional groups that can act as solubilising agents, and that other suitable compounds or functional groups including those that can act as solubilising agents known in the art may be used in accordance with the present invention, such as carbohydrates.

The way in which the one or more compounds or functional group (such as solubilising agents) may be conjugated to a lipid according to the present invention would be well known to a person skilled in the art. For example, conjugation via Fmoc chemistry, through a disulfide or a dioether bridge, or via oxime chemistry is envisaged. In a particular embodiment of the present invention, a soluble form of Pam2Cys was prepared by addition of O—(N-Fmoc-2-aminoethyl)-O′-(2-carboxyethyl)-undecaethyleneglycol (Fmoc-PEOn-OH, Merck Ltd) to Pam2Cys. This resulted in the formation of a PEGylated form of the lipid, Pam2Cys-PEG₁₁ which is then suitable for administration to a subject.

In another form of the invention, the TLR2 moiety comprises a conjugate comprising Pam2Cys conjugated to a pendant R4 form. In a preferred form, pendant-Pam2Cys is conjugated to R4 according to the following structure:

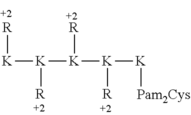

In a preferred form according to any embodiment of the present invention, the TLR2 moiety comprises a conjugate comprising Pam2Cys conjugated to PEG. In a preferred form according to any embodiment of the present invention, the TLR2 moiety comprises a conjugate comprising Pam2Cys conjugated to PEG₁₁. Preferably, the Pam2Cys and PEG₁₁ molecules are separated by at least two serines (PEG₁₁-SS-Pam2Cys).

As used herein, reference to a TLR2 agonist also includes a pharmaceutically acceptable salt, solvate, polymorph or prodrug thereof.

Additional compounds that comprise a TLR2 agonist that are useful in any aspect of the present invention are described below.

In any aspect of the present invention, the compound comprising a TLR2 agonist comprises the structure:

A-Y-B

wherein A comprises or consists of:

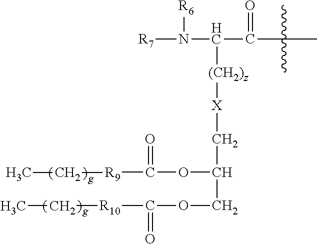

wherein each g is independently 10, 11, 12, 13, 14, 15, 16, 17 or 18;

z is 1 or 2;

X is S or S(═O);

R₆ and R₇ are independently selected from the group consisting of H, a straight or branched C₁-C₄ alkyl, and —C(═O)CH₃;

R₉ and R₁₀ are independently selected from the group consisting of —NH—, —O— or a single bond;

Y is

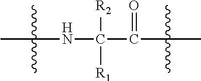

wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;

R₈ is selected from the group consisting of H and a straight or branched C₁-C₆ alkyl;

and

B comprises or consists of Polyethylene Glycol (PEG),

or a pharmaceutically acceptable salt or prodrug thereof.

The term “alkyl” refers to a saturated, straight-chain (i.e. linear) or branched hydrocarbon group. Specific examples of alkyl groups are methyl, ethyl, propyl, iso-propyl, n-butyl, iso-butyl, sec-butyl, tert-butyl, n-pentyl, iso-pentyl, n-hexyl and 2,2-dimethylbutyl. The alkyl group may be a C₁-C₄ or C₁-C₆ alkyl group. As used herein a wording defining the limits of a range of length such as, for example, “from 1 to 5” means any integer from 1 to 5, i.e. 1, 2, 3, 4 and 5. In other words, any range defined by two integers explicitly mentioned is meant to comprise and disclose any integer defining said limits and any integer comprised in said range. The alkyl group may be a branched alkyl group.

In any aspect of the present invention, the compound comprising a TLR2 agonist comprises the structure:

A-Y-B

wherein A comprises or consists of:

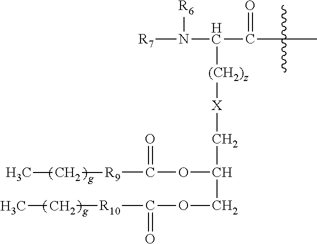

wherein each g is independently 10, 11, 12, 13, 14, 15, 16, 17 or 18;

z is 1 or 2;

X is S or S(═O);

R₆ and R₇ are independently selected from the group consisting of H, a straight or branched C₁-C₄ alkyl, and —C(═O)CH₃;

R₉ and R₁₀ are independently selected from the group consisting of —NH—, —O— or a single bond;

Y is

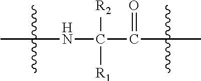

wherein R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₂ are not both H;

and

B comprises or consists of Polyethylene Glycol (PEG),

or a pharmaceutically acceptable salt or prodrug thereof.

In any aspect of the present invention, the compound comprising a TLR2 agonist comprises the structure:

A-Y-B

wherein A comprises or consists of:

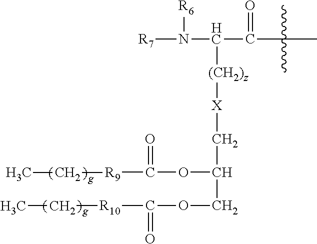

wherein each g is independently 10, 11, 12, 13, 14, 15, 16, 17 or 18;

z is 1;

x is s;

R₆ and R₇ are H;

R₉ and R₁₀ are both a single bond;

Y is

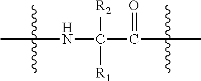

wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;

R₈ is selected from the group consisting of H and a straight or branched C₁-C₆ alkyl;

and

B comprises or consists of Polyethylene Glycol (PEG),

or a pharmaceutically acceptable salt or prodrug thereof.

In any aspect of the present invention, the compound comprising a TLR2 agonist comprises the structure:

A-Y-B

wherein A comprises or consists of:

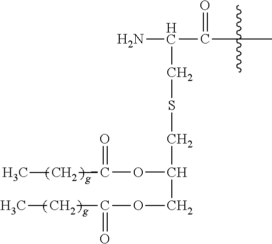

wherein each g is independently 10, 11, 12, 13, 14, 15, 16, 17 or 18;

Y is

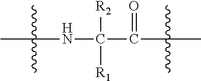

wherein R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₂ are not both H;

and

B comprises or consists of Polyethylene Glycol (PEG),

or a pharmaceutically acceptable salt or prodrug thereof.

In any aspect of the present invention, the compound comprising a TLR2 agonist comprises Pam2Cys and PEG, wherein the Pam2Cys and PEG are linked by a glycine, serine, homoserine, threonine, phosphoserine, asparagine or glutamine residue, or an ester of a glutamine residue,

wherein

Pam2Cys in the compound has the structure:

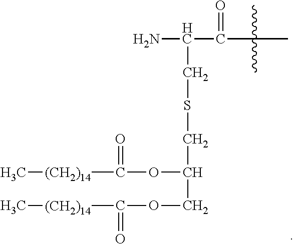

The term “ester” refers to a carboxylic acid group where the hydrogen of the hydroxyl group has been replaced by a saturated, straight-chain (i.e. linear) or branched hydrocarbon group. Specific examples of alkyl groups are methyl, ethyl, propyl, iso-propyl, n-butyl, iso-butyl, sec-butyl, tert-butyl, n-pentyl, iso-pentyl, n-hexyl and 2,2-dimethylbutyl. The alkyl group may be a C₁-C₆ alkyl group. As used herein a wording defining the limits of a range of length such as, for example, “from 1 to 5” means any integer from 1 to 5, i.e. 1, 2, 3, 4 and 5. In other words, any range defined by two integers explicitly mentioned is meant to comprise and disclose any integer defining said limits and any integer comprised in said range. The alkyl group may be a branched alkyl group.

In any aspect of the present invention, the compound comprising a TLR2 agonist comprises Pam2Cys and PEG, wherein the Pam2Cys and PEG are linked by a serine, homoserine, threonine or phosphoserine residue,

wherein

Pam2Cys in the compound has the structure:

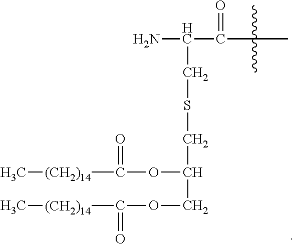

In any aspect of the present invention, the compound comprising a TLR2 agonist comprises:

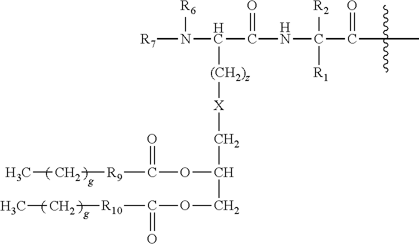

wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;

R₆ and R₇ are independently selected from the group consisting of H, a straight or branched C₁-C₄ alkyl, and —C(═O)CH₃;

R₈ is selected from the group consisting of H and a straight or branched C₁-C₆ alkyl;

R₉ and R₁₀ are independently selected from the group consisting of —NH—, —O— or a single bond;
z is 1 or 2; and

X is S or S(═O);

covalently linked to polyethylene glycol (PEG),

or a pharmaceutically acceptable salt or prodrug thereof.

In any aspect of the present invention, the compound comprising a TLR2 agonist comprises:

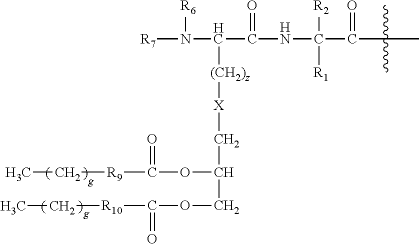

wherein R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₂ are not both H;

R₆ and R₇ are independently selected from the group consisting of H, a straight or branched C₁-C₄ alkyl, and —C(═O)CH₃;

R₉ and R₁₀ are independently selected from the group consisting of —NH—, —O— or a single bond;

z is 1 or 2; and

X is S or S(═O);

covalently linked to polyethylene glycol (PEG),

or a pharmaceutically acceptable salt or prodrug thereof.

In any aspect of the present invention, the compound comprising a TLR2 agonist comprises:

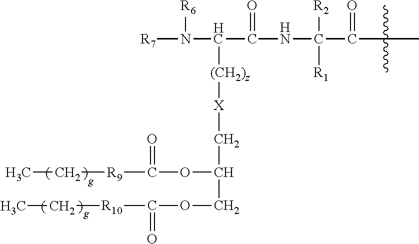

wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;

R₆ and R₇ are H;

R₉ and R₁₀ are both a single bond;

R₈ is selected from the group consisting of H and a straight or branched C₁-C₆ alkyl;

z is 1; and

X is S;

covalently linked to polyethylene glycol (PEG),

or a pharmaceutically acceptable salt or prodrug thereof.

In any aspect of the present invention, the compound comprising a TLR2 agonist comprises:

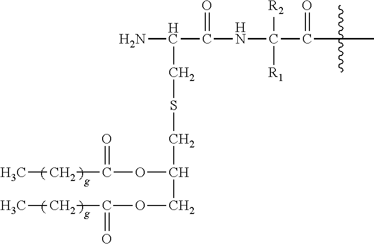

wherein R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₂ are not both H;

covalently linked to polyethylene glycol (PEG),

or a pharmaceutically acceptable salt or prodrug thereof.

In any aspect of the present invention, the compound comprising a TLR2 agonist is a compound of formula (VI):

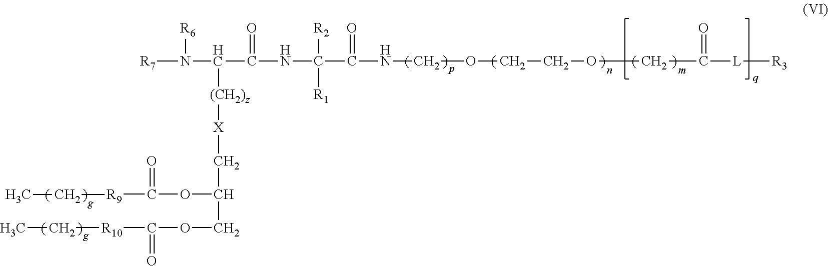

wherein

n is 3 to 100;

m is 1, 2, 3 or 4;

each g is independently 10, 11, 12, 13, 14, 15, 16, 17 or 18;

p is 2, 3 or 4;

q is null or 1;

R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;

R₆ and R₇ are independently selected from the group consisting of H, a straight or branched C₁-C₄ alkyl, and —C(═O)CH₃;

R₈ is selected from the group consisting of H and a straight or branched C₁-C₆ alkyl;

R₉ and R₁₀ are independently selected from the group consisting of —NH—, —O— or a single bond;

z is 1 or 2;

X is S or S(═O);

wherein when q=1, R₃ is —NH₂ or —OH;

wherein when q=0, R₃ is H;

L is null or consists of 1 to 10 units, wherein each unit is a natural alpha amino acid or derived from a natural alpha amino acid, and has the formula:

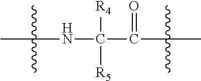

wherein R₄ is H; and

R₅ is the side chain, or second hydrogen of the amino acid

or a pharmaceutically acceptable salt or prodrug thereof.

In any aspect of the present invention, the compound comprising a TLR2 agonist is a compound of formula (VI):

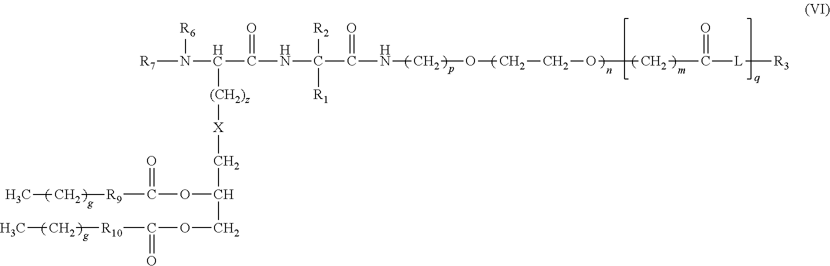

wherein

n is 3 to 100;

m is 1, 2, 3 or 4;

each g is independently 10, 11, 12, 13, 14, 15, 16, 17 or 18;

p is 2, 3 or 4;

q is null or 1;

R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₂ are not both H;

R₆ and R₇ are independently selected from the group consisting of H, a straight or branched C₁-C₄ alkyl, and —C(═O)CH₃;

R₉ and R₁₀ are independently selected from the group consisting of —NH—, —O— or a single bond;

z is 1 or 2;

X is S or S(═O);

wherein when q=1, R₃ is —NH₂ or —OH;

wherein when q=0, R₃ is H;

L is null or consists of 1 to 10 units, wherein each unit is a natural alpha amino acid or derived from a natural alpha amino acid, and has the formula:

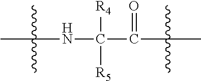

wherein R₄ is H; and

R₅ is the side chain, or second hydrogen of the amino acid or a pharmaceutically acceptable salt or prodrug thereof.

In any aspect of the present invention, the compound comprising a TLR2 agonist is a compound of formula (VI):

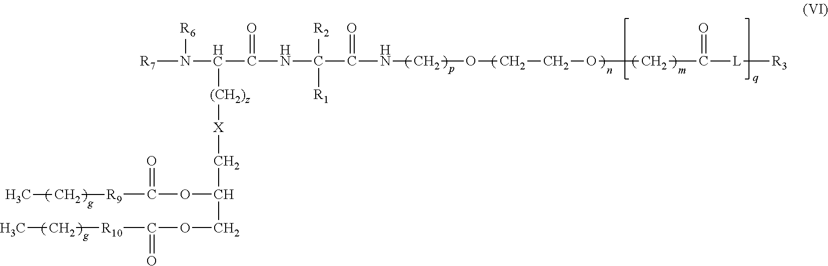

wherein

n is 3 to 100;

m is 1, 2, 3 or 4;

each g is independently 10, 11, 12, 13, 14, 15, 16, 17 or 18;

p is 2, 3 or 4;

q is null or 1;

R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;

R₆ and R₇ are H;

R₉ and R₁₀ are both a single bond;

R₈ is selected from the group consisting of H and a straight or branched C₁-C₆ alkyl;

z is 1;

X is S;

wherein when q=1, R₃ is —NH₂ or —OH;

wherein when q=0, R₃ is H;

L is null or consists of 1 to 10 units, wherein each unit is a natural alpha amino acid or derived from a natural alpha amino acid, and has the formula:

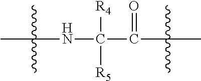

wherein R₄ is H; and

R₅ is the side chain, or second hydrogen of the amino acid

or a pharmaceutically acceptable salt or prodrug thereof.

In any aspect of the present invention, the compound comprising a TLR2 agonist is a compound of formula (I):

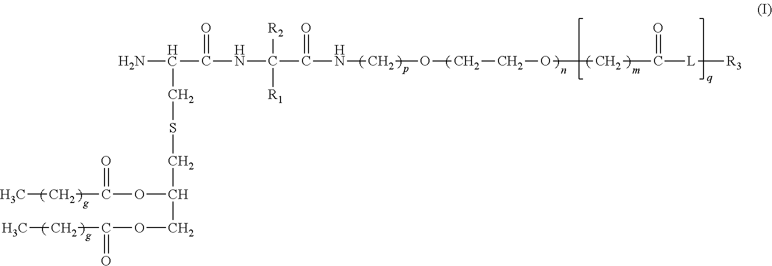

wherein

n is 3 to 100;

m is 1, 2, 3 or 4;

each g is independently 10, 11, 12, 13, 14, 15, 16, 17 or 18;

p is 2, 3 or 4;

q is null or 1;

R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₂ are not both H;

wherein when q=1, R₃ is —NH₂ or —OH;

wherein when q=0, R₃ is H;

L is null or consists of 1 to 10 units, wherein each unit is a natural alpha amino acid or derived from a natural alpha amino acid, and has the formula:

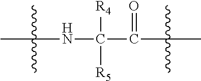

wherein R₄ is H; and

R₅ is the side chain, or second hydrogen of the amino acid

or a pharmaceutically acceptable salt or prodrug thereof.

In any aspect of the present invention, the compound comprising a TLR2 agonist is a compound of formula (VII):

A-Y—NH—(CH₂)ₚ—O—(CH₂—CH₂—O)ₙ—[(CH₂)ₘ—CO-L-]_{q}R₃

wherein

A has the structure:

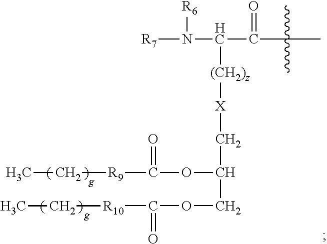

Y is

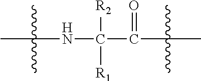

wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;

R₆ and R₇ are independently selected from the group consisting of H, a straight or branched C₁-C₄ alkyl, and —C(═O)CH₃;

R₈ is selected from the group consisting of H and a straight or branched C₁-C₆ alkyl;

R₉ and R₁₀ are independently selected from the group consisting of —NH—, —O— or a single bond;

z is 1 or 2;

X is S or S(═O);

n is 3 to 100;

m is 1, 2, 3 or 4;

each g is independently 10, 11, 12, 13, 14, 15, 16, 17 or 18;

p is 2, 3 or 4;

q is null or 1;

wherein when q=1, R₃ is —NH₂ or —OH;

wherein when q=0, R₃ is H;

L is null or consists of 1 to 10 units, wherein each unit is a natural alpha amino acid or derived from a natural alpha amino acid, and has the formula:

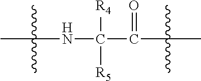

wherein R₄ is H; and

R₅ is the side chain, or second hydrogen of the amino acid,

or a pharmaceutically acceptable salt or prodrug thereof.

In any aspect of the present invention, the compound comprising a TLR2 agonist is a compound of formula (VII):

A-Y—NH—(CH₂)ₚ—O—(CH₂—CH₂—O)ₙ—[(CH₂)ₘ—CO-L-]_{q}R₃

wherein

A has the structure:

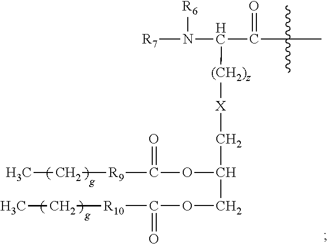

Y is

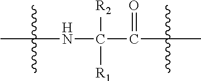

wherein R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₂ are not both H;

R₆ and R₇ are independently selected from the group consisting of H, a straight or branched C₁-C₄ alkyl, and —C(═O)CH₃;

R₉ and R₁₀ are independently selected from the group consisting of —NH—, —O— or a single bond;

z is 1 or 2;

X is S or S(═O);

n is 3 to 100;

m is 1, 2, 3 or 4;

each g is independently 10, 11, 12, 13, 14, 15, 16, 17 or 18;

p is 2, 3 or 4;

q is null or 1;

wherein when q=1, R₃ is —NH₂ or —OH;

wherein when q=0, R₃ is H;

L is null or consists of 1 to 10 units, wherein each unit is a natural alpha amino acid or derived from a natural alpha amino acid, and has the formula:

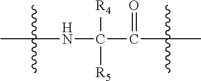

wherein R₄ is H; and

R₅ is the side chain, or second hydrogen of the amino acid, or a pharmaceutically acceptable salt or prodrug thereof.

In any aspect of the present invention, the compound comprising a TLR2 agonist is a compound of formula (VII):

A-Y—NH—(CH₂)ₚ—O—(CH₂—CH₂—O)ₙ—[(CH₂)ₘ—CO-L-]_{q}R₃ (VII)

wherein

A has the structure:

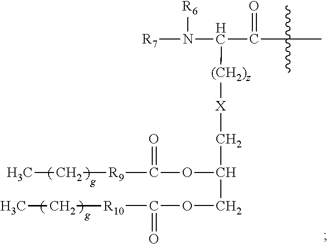

Y is

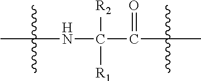

wherein R₁ and R₂ are independently selected from the group consisting of H, —CH₂OH, —CH₂CH₂OH, —CH(CH₃)OH, —CH₂OPO(OH)₂, —CH₂C(═O)N H2, —CH₂CH₂C(═O)OH and —CH₂CH₂C(═O)OR₈, wherein any one of the alkyl hydrogens can be replaced with a halogen;

R₆ and R₇ are H;

R₉ and R₁₀ are both a single bond;

R₈ is selected from the group consisting of H and a straight or branched C₁-C₆ alkyl;

z is 1;

X is S;

n is 3 to 100;

m is 1, 2, 3 or 4;

each g is independently 10, 11, 12, 13, 14, 15, 16, 17 or 18;

p is 2, 3 or 4;

q is null or 1;

wherein when q=1, R₃ is —NH₂ or —OH;

wherein when q=0, R₃ is H;

L is null or consists of 1 to 10 units, wherein each unit is a natural alpha amino acid or derived from a natural alpha amino acid, and has the formula:

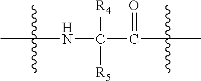

wherein R₄ is H; and

R₅ is the side chain, or second hydrogen of the amino acid,

or a pharmaceutically acceptable salt or prodrug thereof.

In any aspect of the present invention, the compound comprising a TLR2 agonist is a compound of formula (II):

A-Y—NH—(CH₂)ₚ—O—(CH₂—CH₂—O)ₙ—[(CH₂)ₘ—CO-L-]_{q}R₃ (II)

wherein

A has the structure:

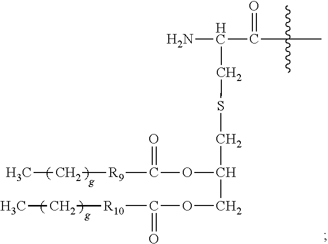

Y is

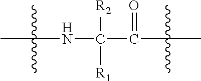

wherein R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₂ are not both H;

n is 3 to 100;

m is 1, 2, 3 or 4;

each g is independently 10, 11, 12, 13, 14, 15, 16, 17 or 18;

p is 2, 3 or 4;

q is null or 1;

wherein when q=1, R₃ is —NH₂ or —OH;

wherein when q=0, R₃ is H;

L is null or consists of 1 to 10 units, wherein each unit is a natural alpha amino acid or derived from a natural alpha amino acid, and has the formula:

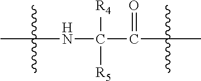

wherein R₄ is H; and

R₅ is the side chain, or second hydrogen of the amino acid,

or a pharmaceutically acceptable salt or prodrug thereof.

In any aspect of the present invention, the compound comprising a TLR2 agonist is a compound of formula (VIII):

Pam2Cys-Y—NH—(CH₂)ₚ—O—(CH₂—CH₂—O)ₙ—[(CH₂)ₘ—CO-L-]_{q}R₃ (VIII)

wherein

Pam2Cys has the structure:

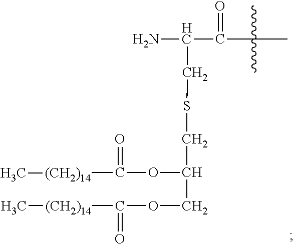

Y is:

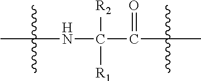

wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;

R₈ is selected from the group consisting of H and a straight or branched C₁-C₆ alkyl;

n is 3 to 100;

m is 1, 2, 3 or 4;

p is 2, 3 or 4;

q is null or 1;

wherein when q=1, R₃ is H, —NH₂ or —OH;

wherein when q=0, R₃ is H;

L is null or consists of 1 to 10 units, wherein each unit is a natural alpha amino acid or derived from a natural alpha amino acid, and has the formula:

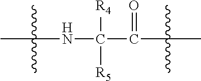

wherein R₄ is H; and

R₅ is the side chain, or second hydrogen of the amino acid,

or a pharmaceutically acceptable salt or prodrug thereof.

In any aspect of the present invention, the compound comprising a TLR2 agonist is a compound of formula (III):

Pam2Cys-Y—NH—(CH₂)ₚ—O—(CH₂—CH₂—O)ₙ—[(CH₂)ₘ—CO-L-]_{q}R₃ (III)

wherein

Pam2Cys has the structure:

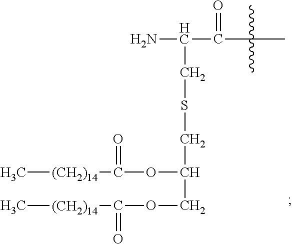

Y is:

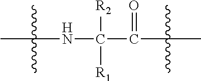

wherein R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₂ are not both H;

n is 3 to 100;

m is 1, 2, 3 or 4;

p is 2, 3 or 4;

q is null or 1;

wherein when q=1, R₃ is H, —NH₂ or —OH;

wherein when q=0, R₃ is H;

L is null or consists of 1 to 10 units, wherein each unit is a natural alpha amino acid or derived from a natural alpha amino acid, and has the formula:

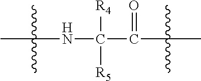

wherein R₄ is H; and

R₅ is the side chain, or second hydrogen of the amino acid,

or a pharmaceutically acceptable salt or prodrug thereof.

In any aspect of the present invention, the compound comprising a TLR2 agonist is a compound of formula (IV):

Pam2Cys-Ser-NH—(CH₂)ₚ—O—(CH₂—CH₂—O)ₙ—[(CH₂)ₘ—CO-L-]_{q}R₃ (IV)

wherein

Pam2Cys-Ser has the structure:

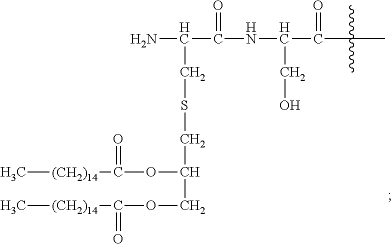

n is 3 to 100;

m is 1, 2, 3 or 4;

p is 2, 3 or 4;

q is null or 1;

wherein when q=1, R₃ is —NH₂ or —OH;

wherein when q=0, R₃ is H;

L is null or consists of 1 to 10 units, wherein each unit is a natural alpha amino acid or derived from a natural alpha amino acid, and has the formula:

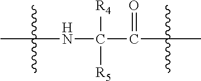

wherein R₄ is H; and

R₅ is the side chain, or second hydrogen of the amino acid,

or a pharmaceutically acceptable salt or prodrug thereof.

In any aspect of the present invention, the compound comprising a TLR2 agonist is a compound of formula (X):

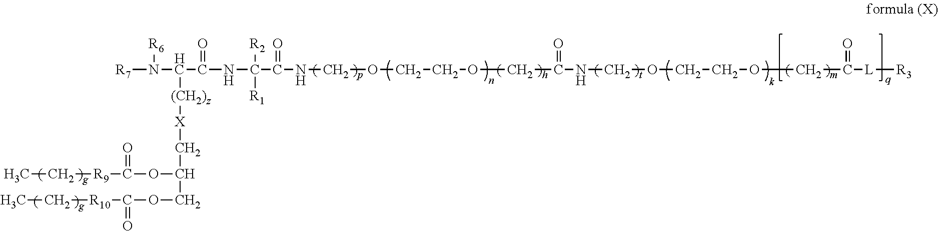

wherein

n is 3 to 100;

k is 3 to 100;

m is 1, 2, 3 or 4;

each g is independently 10, 11, 12, 13, 14, 15, 16, 17 or 18;

p is 2, 3 or 4;

t is 2, 3 or 4;

h is 1, 2, 3 or 4;

q is null or 1;

wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;

R₆ and R₇ are independently selected from the group consisting of H, a straight or branched C₁-C₄ alkyl, and —C(═O)CH₃;

R₈ is selected from the group consisting of H and a straight or branched C₁-C₆ alkyl;

R₉ and R₁₀ are independently selected from the group consisting of —NH—, —O— or a single bond;

z is 1 or 2;

X is S or S(═O);

wherein when q=1, R₃ is —NH₂ or —OH;

wherein when q=0, R₃ is H;

L is null or consists of 1 to 10 units, wherein each unit is a natural alpha amino acid or derived from a natural alpha amino acid, and has the formula:

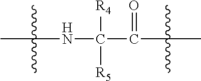

wherein R₄ is H; and

R₅ is the side chain, or second hydrogen of the amino acid,

or a pharmaceutically acceptable salt or prodrug thereof.

In any aspect of the present invention, the compound comprising a TLR2 agonist is a compound of formula (X):

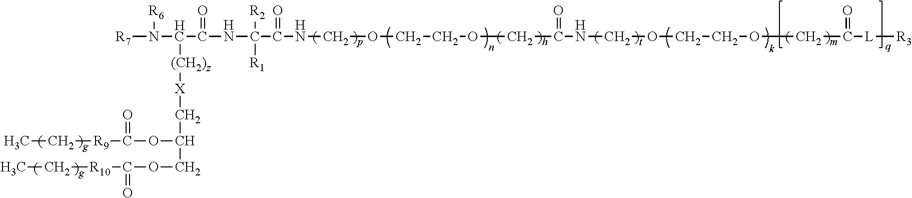

wherein

n is 3 to 100;

k is 3 to 100;

m is 1, 2, 3 or 4;

each g is independently 10, 11, 12, 13, 14, 15, 16, 17 or 18;

p is 2, 3 or 4;

t is 2, 3 or 4;

h is 1, 2, 3 or 4;

q is null or 1;

wherein R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₂ are not both H;

R₆ and R₇ are independently selected from the group consisting of H, a straight or branched C₁-C₄ alkyl, and —C(═O)CH₃;

R₉ and R₁₀ are independently selected from the group consisting of —NH—, —O— or a single bond;

z is 1 or 2;

X is S or S(═O);

wherein when q=1, R₃ is —NH₂ or —OH;

wherein when q=0, R₃ is H;

L is null or consists of 1 to 10 units, wherein each unit is a natural alpha amino acid or derived from a natural alpha amino acid, and has the formula:

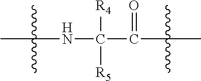

wherein R₄ is H; and

R₅ is the side chain, or second hydrogen of the amino acid,

or a pharmaceutically acceptable salt or prodrug thereof.

In any aspect of the present invention, the compound comprising a TLR2 agonist is a compound of formula (X):

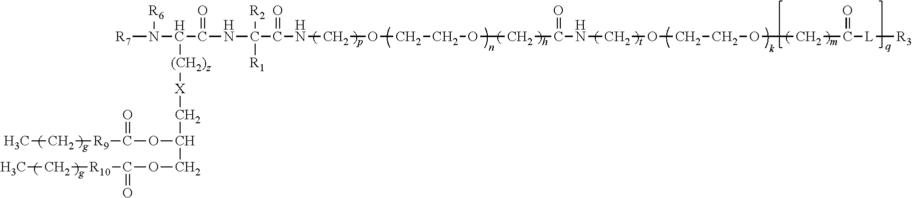

wherein

n is 3 to 100;

k is 3 to 100;

m is 1, 2, 3 or 4;

each g is independently 10, 11, 12, 13, 14, 15, 16, 17 or 18;

p is 2, 3 or 4;

t is 2, 3 or 4;

h is 1, 2, 3 or 4;

q is null or 1;

wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;

R₆ and R₇ are H;

R₉ and R₁₀ are both a single bond;

R₈ is selected from the group consisting of H and a straight or branched C₁-C₆ alkyl;

z is 1;

X is S;

wherein when q=1, R₃ is —NH₂ or —OH;

wherein when q=0, R₃ is H;

L is null or consists of 1 to 10 units, wherein each unit is a natural alpha amino acid or derived from a natural alpha amino acid, and has the formula:

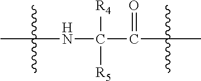

wherein R₄ is H; and

R₅ is the side chain, or second hydrogen of the amino acid,

or a pharmaceutically acceptable salt or prodrug thereof.

In any aspect of the present invention, the compound comprising a TLR2 agonist is a compound of formula (V):

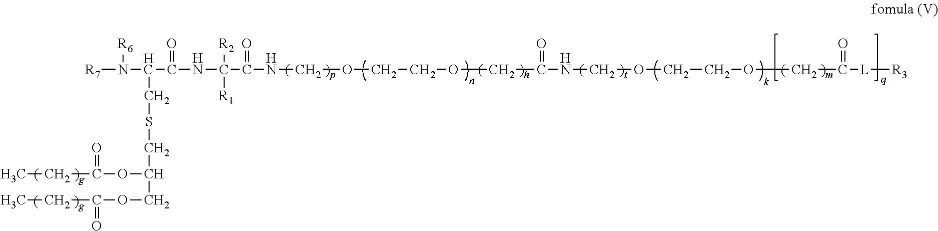

wherein

n is 3 to 100;

k is 3 to 100;

m is 1, 2, 3 or 4;

each g is independently 10, 11, 12, 13, 14, 15, 16, 17 or 18;

p is 2, 3 or 4;

t is 2, 3 or 4;

h is 1, 2, 3 or 4;

q is null or 1;

R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₂ are not both H;

wherein when q=1, R₃ is —NH₂ or —OH;

wherein when q=0, R₃ is H;

L is null or consists of 1 to 10 units, wherein each unit is a natural alpha amino acid or derived from a natural alpha amino acid, and has the formula:

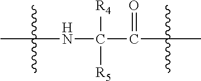

wherein R₄ is H; and

R₅ is the side chain, or second hydrogen of the amino acid,

or a pharmaceutically acceptable salt or prodrug thereof.

In any aspect of the present invention, the compound comprising a TLR2 agonist is a compound comprising a chiral centre around the following chiral centre (shown at *):

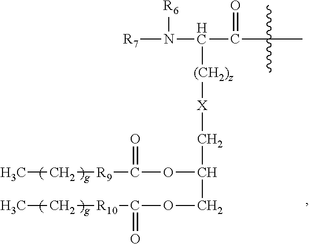

wherein the chiral centre is in the R configuration. A compound in this form may also be referred to as an R-Pam2 analogue diastereomer of the compound. This may be depicted as:

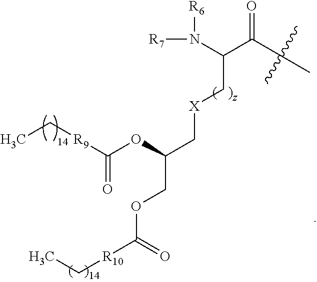

In any aspect of the present invention, the compound comprising a TLR2 agonist is a compound comprising a chiral centre in the 2,3-bis(palmitoyloxy)propyl moiety of Pam2Cys (shown at *):

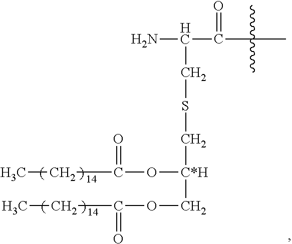

wherein the chiral centre is in the R configuration. A compound in this form may also be referred to as an R-Pam2 diastereomer of the compound. This may be depicted as:

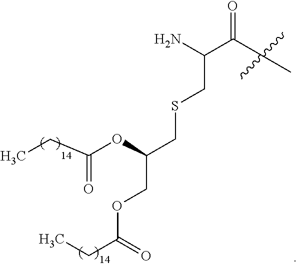

In any aspect of the present invention, the compound comprising a TLR2 agonist is a compound comprising a chiral centre around the following chiral centre (shown at *):

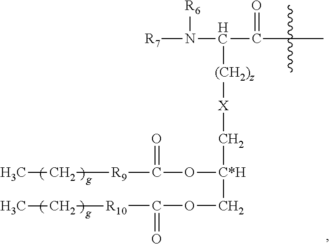

wherein the chiral centre is in the S configuration. A compound in this form may also be referred to as an S-Pam2 analogue diastereomer of the compound. This may be depicted as:

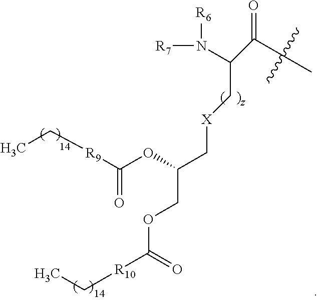

In any aspect of the present invention, the compound comprising a TLR2 agonist is a compound comprising a chiral centre in the 2,3-bis(palmitoyloxy)propyl moiety of Pam2Cys (shown at *):

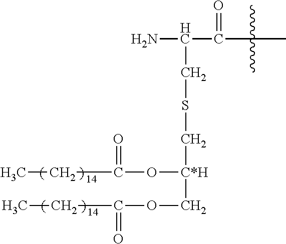

wherein the chiral centre is in the S configuration. A compound in this form may also be referred to as an S-Pam2 diastereomer of the compound. This may be depicted as:

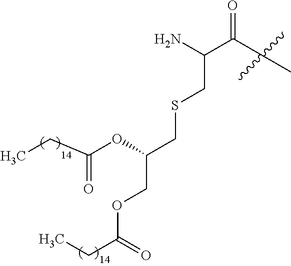

In any aspect of the present invention, the compound comprising a TLR2 agonist is a compound comprising a chiral centre around the following chiral centre (shown at *):

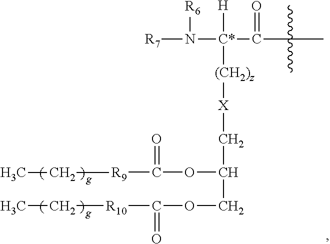

wherein the chiral centre is in the L configuration. A compound in this form may also be referred to as an L-Cys analogue diastereomer of Pam2Cys of the compound. This may be depicted as:

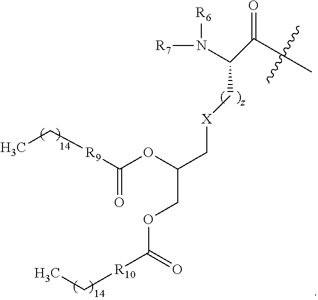

In any aspect of the present invention, the compound comprising a TLR2 agonist is a compound comprising a chiral centre in the cysteine residue of Pam2Cys (shown at *):

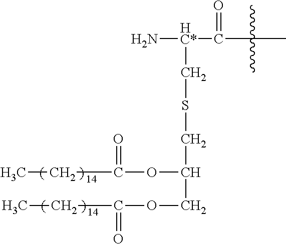

wherein the chiral centre is in the L configuration. A compound in this form may also be referred to as an L-Cys diastereomer of Pam2Cys of the compound. This may be depicted as:

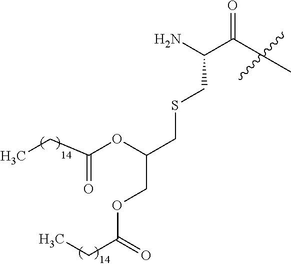

In any aspect of the present invention, the compound comprising a TLR2 agonist is a compound comprising a chiral centre around the following chiral centre (shown at *):

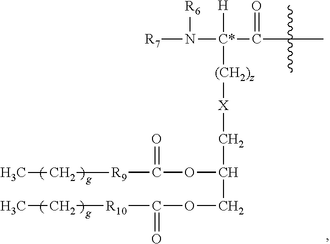

wherein the chiral centre is in the D configuration. A compound in this form may also be referred to as an D-Cys analogue diastereomer of Pam2Cys of the compound. This may be depicted as:

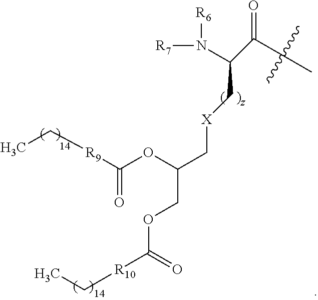

In any aspect of the present invention, the compound comprising a TLR2 agonist is a compound comprising a chiral centre in the cysteine residue of Pam2Cys (shown at *):

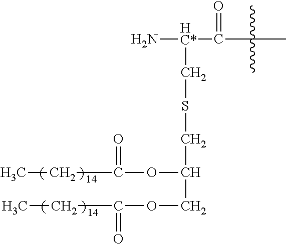

wherein the chiral centre is in the D configuration. A compound in this form may also be referred to as a D-Cys diastereomer of Pam2Cys of the compound. This may be depicted as:

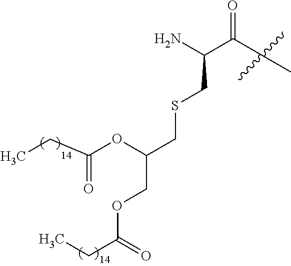

In any aspect of the present invention, the compound comprising a TLR2 agonist is a compound comprising a chiral centre in the Y moiety of the compound (shown at *):

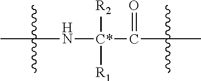

wherein the chiral centre is in the L-configuration. A compound in this form may also be referred to as an L-Y diastereomer of the compound.

In any aspect of the present invention, the compound comprising a TLR2 agonist is a compound comprising a chiral centre in the Y moiety of the compound (shown at *):

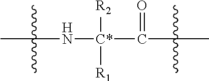

wherein the chiral centre is in the D-configuration. A compound in this form may also be referred to as an D-Y diastereomer of the compound.

In one preferred embodiment, the compound has the structure of compound (1):

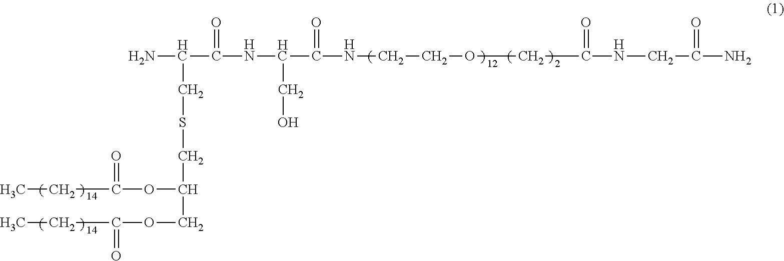

or a pharmaceutically acceptable salt or prodrug thereof.

This compound may also be referred to herein as Pam2Cys-Ser-PEG′, or ‘INNA-006’.

In other preferred embodiments, the compound is selected from the group consisting of:

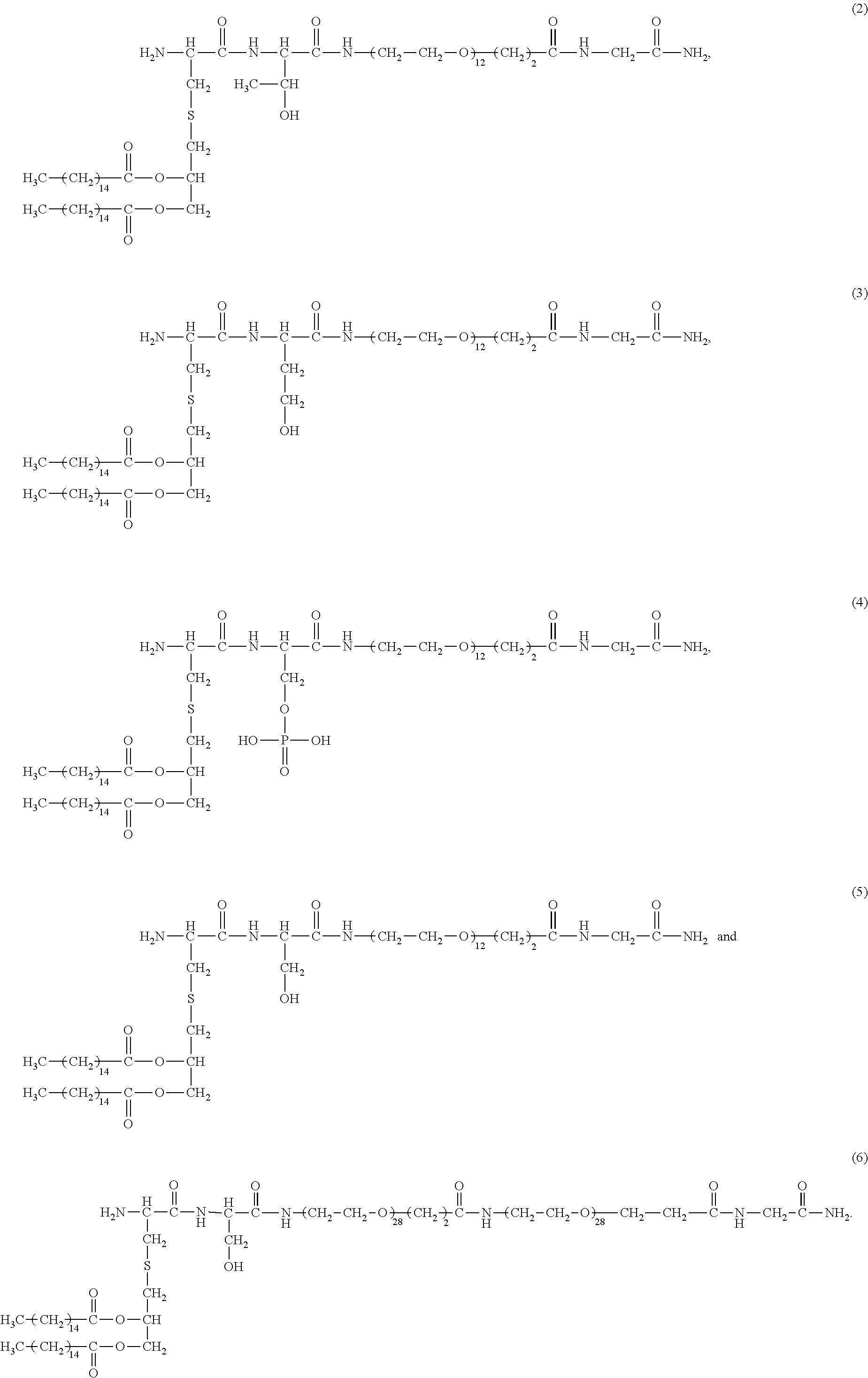 [SMILES: CCC(=O)OCC(CSCC(N)C(=O)NC(C(=O)NCCOCC(=O)NCC(N)=O)C(C)O)OC(=O)CC ; CCC(=O)OCC(CSCC(N)C(=O)NC(CCO)C(=O)NCCOCC(=O)NCC(N)=O)OC(=O)CC ; CCC(=O)OCC(CSCC(N)C(=O)NC(COP(=O)(O)O)C(=O)NCCOCC(=O)NCC(N)=O)OC(=O)CC ; CCC(=O)OCC(CSCC(N)C(=O)NC(CO)C(=O)NCCOCC(=O)NCC(N)=O)OC(=O)CC ; "and" ; CCC(=O)OCC(CSCC(N)C(=O)NC(CO)C(=O)NCCOCC(=O)NCCOCCC(=O)NCC(N)=O)OC(=O)CC]

In one particularly preferred embodiment, the compound is:

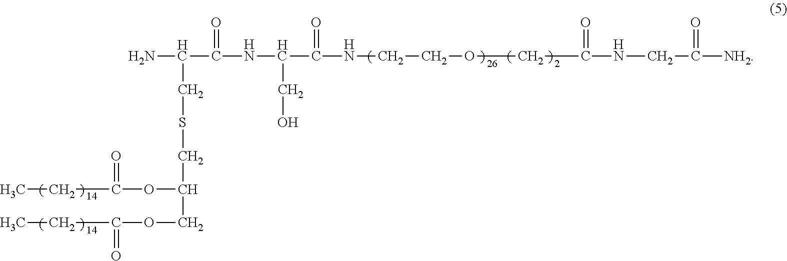

In any aspect of the present invention, the compound comprising a TLR2 agonist is a compound of formula (Ia):

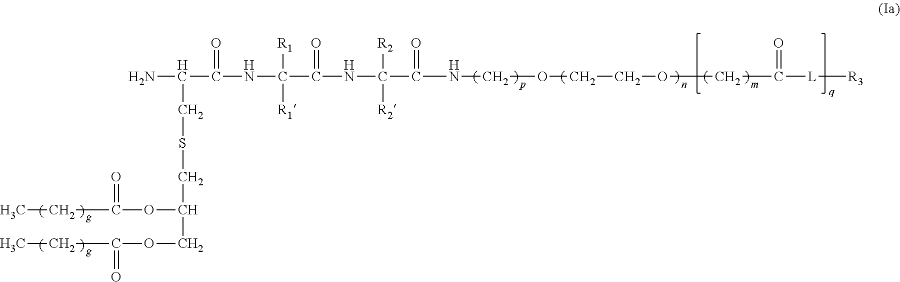

wherein

n is 3 to 100;

m is 1, 2, 3 or 4;

each g is independently 10, 11, 12, 13, 14, 15, 16, 17 or 18;

p is 2, 3 or 4;

q is null or 1;

R₁, R₁′, R₂ and R₂′ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₁′ are not both H, and R₂ and R₂′ are not both H;

wherein when q is null, R₃ is H;

wherein when q is 1, R₃ is —NH₂ or —OH;

L is null or consists of 1 to 10 units, wherein each unit is a natural alpha amino acid or derived from a natural alpha amino acid, and has the formula:

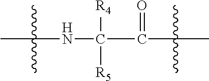

wherein R₄ is H; and

R₅ is the side chain, or second hydrogen of the amino acid,

or a pharmaceutically acceptable salt or prodrug thereof.

In any aspect of the present invention, the compound comprising a TLR2 agonist is a compound of formula (IIa):

A-Y—NH—(CH₂)ₚ—O—(CH₂—CH₂—O)ₙ—[(CH₂)ₘ13 CO-L-]_{q}R₃ (IIa)

wherein

A has the structure:

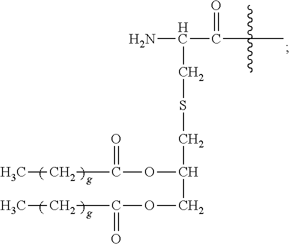

Y is

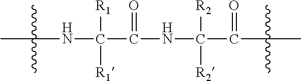

wherein R₁, R₁′, R₂ and R₂′ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₁′ are not both H, and R₂ and R₂′ are not both H;

n is 3 to 100;

m is 1, 2, 3 or 4;

each g is independently 10, 11, 12, 13, 14, 15, 16, 17 or 18;

p is 2, 3 or 4;

q is null or 1;

wherein when q is null, R₃ is H;

wherein when q is 1, R₃ is —NH₂ or —OH;

L is null or consists of 1 to 10 units, wherein each unit is a natural alpha amino acid or derived from a natural alpha amino acid, and has the formula:

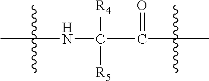

wherein R₄ is H; and

R₅ is the side chain, or second hydrogen of the amino acid,

or a pharmaceutically acceptable salt or prodrug thereof.

In any aspect of the present invention, the compound comprising a TLR2 agonist is a compound of formula (IIIa):

Pam2Cys-Y—NH—(CH₂)ₚ—O—(CH₂—CH₂-0)ₙ—[(CH₂)ₘ_CO-L-]_{q}R₃ (IIIa)

wherein

Pam2Cys has the structure:

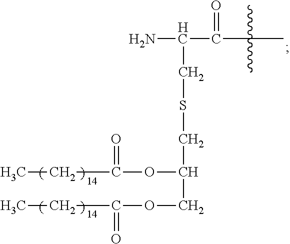

Y is

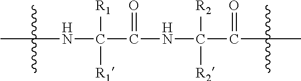

wherein R₁, R₁′, R₂ and R₂′ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₁′ are not both H, and R₂ and R₂′ are not both H;

n is 3 to 100;

m is 1, 2, 3 or 4;

p is 2, 3 or 4;

q is null or 1;

wherein when q is null, R₃ is H;

wherein when q is 1, R₃ is —NH₂ or —OH;

L is null or consists of 1 to 10 units, wherein each unit is a natural alpha amino acid or derived from a natural alpha amino acid, and has the formula:

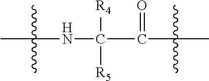

wherein R₄ is H; and

R₅ is the side chain, or second hydrogen of the amino acid,

or a pharmaceutically acceptable salt or prodrug thereof.

In any aspect of the present invention, the compound comprising a TLR2 agonist is a compound of formula (IVa):

Pam2Cys-Ser-Ser-NH—(CH₂)ₚ—O—(CH₂—CH₂—O)ₙ—[(CH₂)ₘ—CO-L-]_{q}R₃ (IVa)

wherein

Pam2Cys has the structure:

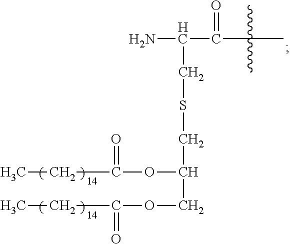

n is 3 to 100;

m is 1, 2, 3 or 4;

p is 2, 3 or 4;

q is null or 1;

R₁, R₁′, R₂ and R₂′ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₁′ are not both H, and R₂ and R₂′ are not both H;

wherein when q is null, R₃ is H;

wherein when q is 1, R₃ is —NH₂ or —OH;

L is null or consists of 1 to 10 units, wherein each unit is a natural alpha amino acid or derived from a natural alpha amino acid, and has the formula:

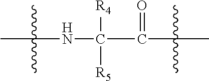

wherein R₄ is H; and

R₅ is the side chain, or second hydrogen of the amino acid, or a pharmaceutically acceptable salt or prodrug thereof.

In any aspect of the present invention, the compound comprising a TLR2 agonist is a compound of formula (Va):

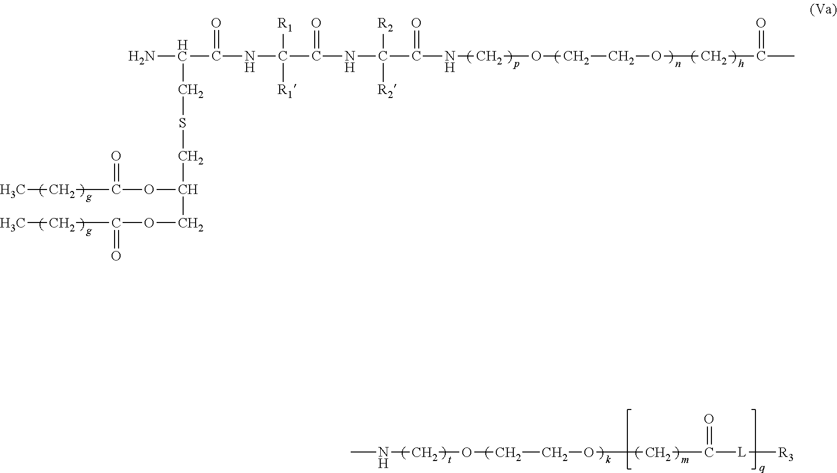

wherein

n is 3 to 100;

k is 3 to 100;

h is 1, 2, 3 or 4;

m is 1, 2, 3 or 4;

each g is independently 10, 11, 12, 13, 14, 15, 16, 17 or 18;

p is 2, 3 or 4;

t is 2, 3 or 4;

q is null or 1;

R₁, R₁′, R₂ and R₂′ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₁′ are not both H, and R₂ and R₂′ are not both H;

wherein when q is null, R₃ is H;

wherein when q is 1, R₃ is —NH₂ or —OH;

L is null or consists of 1 to 10 units, wherein each unit is a natural alpha amino acid or derived from a natural alpha amino acid, and has the formula:

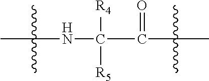

wherein R₄ is H; and

R₅ is the side chain, or second hydrogen of the amino acid,

or a pharmaceutically acceptable salt or prodrug thereof.

In one embodiment, the compound has the structure:

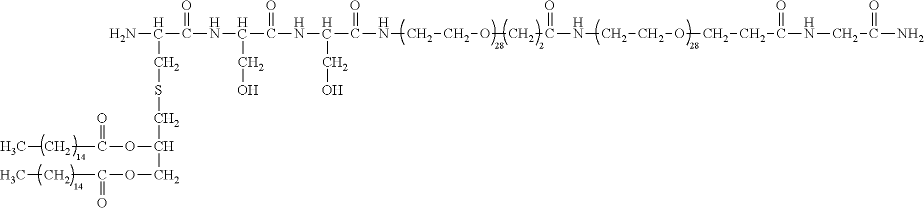

In a particularly preferred embodiment of the invention, the compound has the structure of compound (1a):

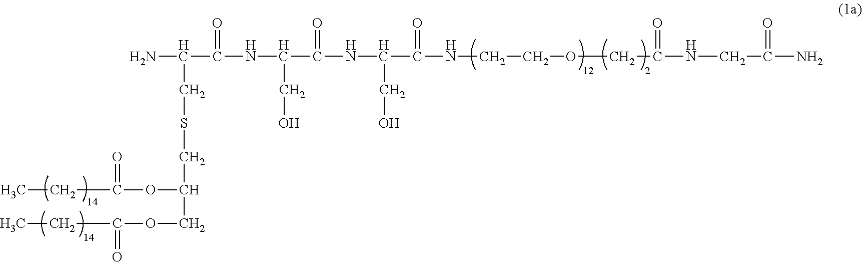

or a pharmaceutically acceptable salt or prodrug thereof.

In other preferred embodiments, the compound is selected from the group consisting of:

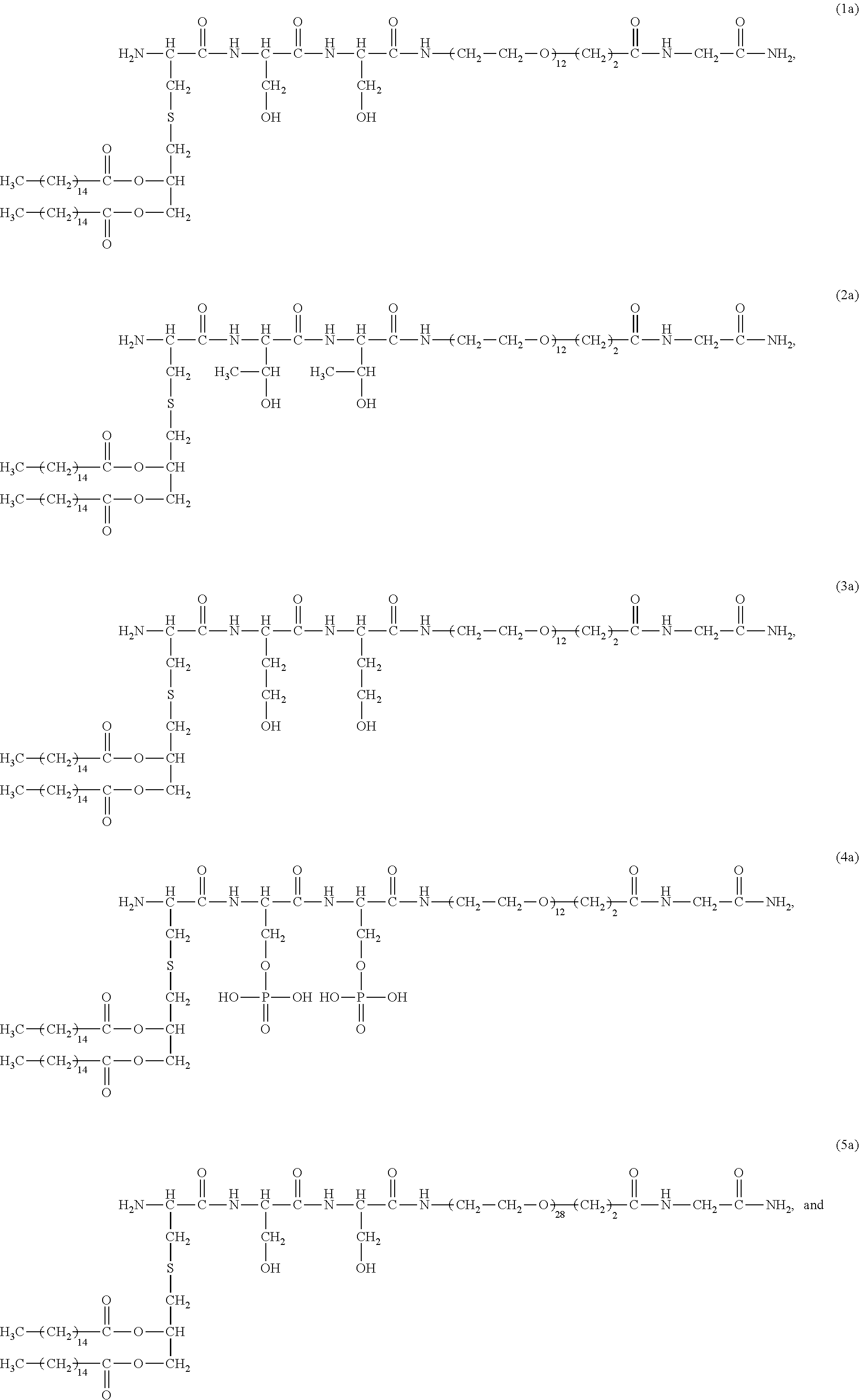 [SMILES: CCC(=O)OCC(CSCC(N)C(=O)NC(CO)C(=O)NC(CO)C(=O)NCCOCC(=O)NCC(N)=O)OC(=O)CC ; CCC(=O)OCC(CSCC(N)C(=O)NC(C(=O)NC(C(=O)NCCOCC(=O)NCC(N)=O)C(C)O)C(C)O)OC(=O)CC ; CCC(=O)OCC(CSCC(N)C(=O)NC(CCO)C(=O)NC(CCO)C(=O)NCCOCC(=O)NCC(N)=O)OC(=O)CC ; CCC(=O)OCC(CSCC(N)C(=O)NC(COP(=O)(O)O)C(=O)NC(COP(=O)(O)O)C(=O)NCCOCC(=O)NCC(N)=O)OC(=O)CC ; CCC(=O)OCC(CSCC(N)C(=O)NC(CO)C(=O)NC(CO)C(=O)NCCOCC(=O)NCC(N)=O)OC(=O)CC ; "and"]

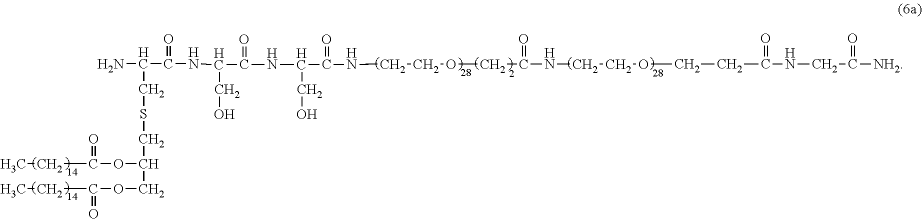 [SMILES: CCC(=O)OCC(CSCC(N)C(=O)NC(CO)C(=O)NC(CO)C(=O)NCCOCC(=O)NCCOCCC(=O)NCC(N)=O)OC(=O)CC]

Also included as compounds of the invention are pharmaceutically acceptable salts or prodrugs of compounds (1) to (6), or (1a) to (6a) above.

For all the above structures, where present, one or more of the following features are preferable:

n is between 10-14, even more preferably, n is 11.

n is 3 or 5.

n is between 24-30, even more preferably, n is 27.

k is between 24-30, even more preferably, k is 27.

m is 1-3, even more preferably, m is 2.

h is 1-3, even more preferably, h is 2.

g is between 10-16, even more preferably, g is between 12-14, most preferably, g is 14.

one of R₁ and R₂ is hydrogen.

p is 2.

t is 2.

z is 1.

X is S.

R₆ and R₇ are H.

R₉ and R₁₀ are both a single bond.

The term “pharmaceutically acceptable” may be used to describe any pharmaceutically acceptable salt, hydrate or prodrug, or any other compound which upon administration to a subject, is capable of providing (directly or indirectly) a compound of the invention as described herein, or a pharmaceutically acceptable salt, prodrug or ester thereof, or an active metabolite or residue thereof.

Suitable pharmaceutically acceptable salts may include, but are not limited to, salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, malic, citric, lactic, mucic, gluconic, benzoic, succinic, oxalic, phenylacetic, methanesulphonic, toluenesulphonic, benzenesulphonic, salicylic, sulphanilic, aspartic, glutamic, edetic, stearic, palmitic, oleic, lauric, pantothenic, tannic, ascorbic and valeric acids.

Base salts may include, but are not limited to, those formed with pharmaceutically acceptable cations, such as sodium, potassium, lithium, calcium, magnesium, zinc, ammonium, alkylammonium such as salts formed from triethylamine, alkoxyammonium such as those formed with ethanolamine and salts formed from ethylenediamine, choline or amino acids such as arginine, lysine or histidine. General information on types of pharmaceutically acceptable salts and their formation is known to those skilled in the art and is as described in general texts such as “Handbook of Pharmaceutical salts” P. H. Stahl, C. G. Wermuth, 1st edition, 2002, Wiley-VCH.

In the case of compounds that are solids, it will be understood by those skilled in the art that the inventive compounds, agents and salts may exist in different crystalline or polymorphic forms, all of which are intended to be within the scope of the present invention and specified formulae.

The term “polymorph” includes any crystalline form of compounds of the invention as described herein, such as anhydrous forms, hydrous forms, solvate forms and mixed solvate forms.

Compounds of the invention described herein are intended to cover, where applicable, solvated as well as unsolvated forms of the compounds. Thus compounds of the invention described herein include compounds having the indicated structures, including the hydrated or solvated forms, as well as the non-hydrated and non-solvated forms.

As used herein, the term “solvate” refers to a complex of variable stoichiometry formed by a solute (in this invention, a compound of the invention described herein, or a pharmaceutically acceptable salt, prodrug or ester thereof) and a solvent. Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent. Examples of suitable pharmaceutically acceptable solvents include, without limitation, water, ethanol and acetic acid. Most preferably the solvent used is water.

Basic nitrogen-containing groups may be quarternised with such agents as lower alkyl halide, such as methyl, ethyl, propyl, and butyl chlorides, bromides and iodides; dialkyl sulfates like dimethyl and diethyl sulfate; and others.

The compounds as described herein are to also include isotope variations, such as the replacement of hydrogen for deuterium.

Compounds of the present invention may exist in and be isolated in optically active and racemic forms. As would be understood by a person skilled in the art, the present invention is intended to encompass any racemic, optically active or stereoisomeric form, or mixtures thereof, of compounds of Formula (I), (II), (III), (IV), (V), (Ia), (IIa), (IIIa), (IVa), (Va), (VI), (VII), (VIII) and/or (X) which possess the useful properties described herein. It is well known in the art how to prepare such forms (for example, by resolution of racemic mixtures by recrystallization, by synthesis from optically-active starting materials, by chiral synthesis, or by chiral chromatographic separation). In one preferred embodiment, with regard to the carbons shown with a * below, the compound of the present invention is provided in a racemic mixture. In another preferred aspect, the compound of the present invention is provided with provided with excess of, or only, the L-configuration or naturally occurring amino acid:

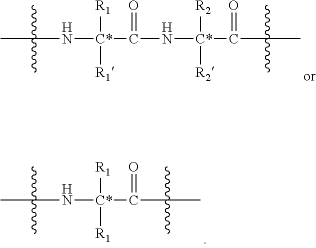

In any aspect of the present invention, the compound comprising a TLR2 agonist as described herein is the L diastereomer around the chiral centre of the cysteine analogue residue of the Pam2Cys analogue moiety compound.

In any aspect of the present invention, the compound comprising a TLR2 agonist as described herein is the L diastereomer around the chiral centre of the cysteine residue of the Pam2Cys moiety compound.

In any aspect of the present invention, the compound comprising a TLR2 agonist as described herein is the D diastereomer around the chiral centre of the cysteine analogue residue of the Pam2Cys analogue moiety compound.

In any aspect of the present invention, the compound comprising a TLR2 agonist as described herein is the D diastereomer around the chiral centre of the cysteine residue of the Pam2Cys moiety of the compound.

In any aspect of the present invention, a composition comprising a TLR2 agonist as described herein comprises a compound that is the L diastereomer around the chiral centre of the cysteine analogue residue of the Pam2Cys analogue moiety of the compound.

In any aspect of the present invention, a composition comprising a TLR2 agonist as described herein comprises a compound that is the L diastereomer around the chiral centre of the cysteine residue of the Pam2Cys moiety of the compound.

In any aspect of the present invention, a composition comprising a TLR2 agonist as described herein comprises a compound that is the D diastereomer around the chiral centre of the cysteine analogue residue of the Pam2Cys analogue moiety of the compound.

In any aspect of the present invention, a composition comprising a TLR2 agonist as described herein comprises a compound that is the D diastereomer around the chiral centre of the cysteine residue of the Pam2Cys moiety of the compound.

In any aspect of the present invention, 1%, 5%, 10%, 20%, 30%, 40%, 50%, 60%, 70%, 80%, 90%, 95%, 96%, 97%, 98%, 99% or more than 99% of the compound present in the composition is the L diastereomer around the chiral centre of the cysteine analogue residue of the Pam2Cys analogue moiety of the compound.

In any aspect of the present invention, 1%, 5%, 10%, 20%, 30%, 40%, 50%, 60%, 70%, 80%, 90%, 95%, 96%, 97%, 98%, 99% or more than 99% of the compound present in the composition is the L diastereomer around the chiral centre of the cysteine residue of the Pam2Cys moiety of the compound.

In any aspect of the present invention, 1%, 5%, 10%, 20%, 30%, 40%, 50%, 60%, 70%, 80%, 90%, 95%, 96%, 97%, 98%, 99% or more than 99% of the compound present in the composition is the D diastereomer around the chiral centre of the cysteine analogue residue of the Pam2Cys analogue moiety of the compound.

In any aspect of the present invention, 1%, 5%, 10%, 20%, 30%, 40%, 50%, 60%, 70%, 80%, 90%, 95%, 96%, 97%, 98%, 99% or more than 99% of the compound present in the composition is the D diastereomer around the chiral centre of the cysteine residue of the Pam2Cys moiety of the compound.

In any aspect of the present invention, the compound comprising a TLR2 agonist as described herein is the L diastereomer around the chiral centre of the Y moiety.

In any aspect of the present invention, the compound comprising a TLR2 agonist as described herein is the D diastereomer around the chiral centre of the Y moiety.

In any aspect of the present invention, a composition comprising a TLR2 agonist as described herein comprises a compound that is the L diastereomer around the chiral centre of the Y moiety.

In any aspect of the present invention, a composition comprising a TLR2 agonist as described herein comprises a compound that is the D diastereomer around the chiral centre of the Y moiety.

In any aspect of the present invention, 1%, 5%, 10%, 20%, 30%, 40%, 50%, 60%, 70%, 80%, 90%, 95%, 96%, 97%, 98%, 99% or more than 99% of the compound present in the composition is the L diastereomer around the chiral centre of the Y moiety.

In any aspect of the present invention, 1%, 5%, 10%, 20%, 30%, 40%, 50%, 60%, 70%, 80%, 90%, 95%, 96%, 97%, 98%, 99% or more than 99% of the compound present in the composition is the D diastereomer around the chiral centre of the Y moiety.

A “prodrug” is a compound that may not fully satisfy the structural requirements of the compounds provided herein, but is modified in vivo, following administration to a subject or patient, to produce a compound of the invention as described herein. For example, a prodrug may be an acylated derivative of a compound as provided herein. Prodrugs include compounds wherein hydroxy, carboxy, amine or sulfhydryl groups are bonded to any group that, when administered to a mammalian subject, cleaves to form a free hydroxy, carboxy, amino, or sulfhydryl group, respectively. Examples of prodrugs include, but are not limited to, acetate, formate, phosphate and benzoate derivatives of alcohol and amine functional groups within the compounds provided herein. Prodrugs of the compounds provided herein may be prepared by modifying functional groups present in the compounds in such a way that the modifications are cleaved in vivo to generate the parent compounds.

Prodrugs include compounds wherein an amino acid residue, or a polypeptide chain of two or more (eg, two, three or four) amino acid residues which are covalently joined to free amino, and amido groups of compounds of Formula (I), (II), (III), (IV), (V), (Ia), (IIa), (IIIa), (IVa), (Va), (VI), (VII), (VIII) and/or (X). The amino acid residues include the 20 naturally occurring amino acids commonly designated by three letter symbols and also include, 4-hydroxyproline, hydroxylysine, demosine, isodemosine, 3-methylhistidine, norvlin, beta-alanine, gamma-aminobutyric acid, citrulline, homocysteine, homoserine, ornithine and methionine sulfone. Prodrugs also include compounds wherein carbonates, carbamates, amides and alkyl esters which are covalently bonded to the above substituents of Formula (I), (II), (III), (IV), (V), (Ia), (IIa), (IIIa), (IVa), (Va), (VI), (VII), (VIII) and/or (X), or other structure as depicted herein.

Administration, Dosage and Formulation

The term ‘respiratory’ refers to the process by which oxygen is taken into the body and carbon dioxide is discharged, through the bodily system including the nose, throat, larynx, trachea, bronchi and lungs.

As used herein, the upper respiratory tract may include the following regions: nose and nasal passages, paranasal sinuses, the pharynx, and the portion of the larynx above the vocal folds (cords).

Typically, the lower respiratory tract includes the following regions: portion of the larynx below the vocal folds, trachea, bronchi and bronchioles. The lungs can be included in the lower respiratory tract and include the respiratory bronchioles, alveolar ducts, alveolar sacs, and alveoli. In any aspect of the present invention, administration to the URT may be administration to the nose and nasal passages, paranasal sinuses, the pharynx, and the portion of the larynx above the vocal folds (cords). Also contemplated is administration to any one or more regions of the URT provided that the compound is retained in the URT, or does not contact a region of the LRT.

The term ‘respiratory disease’ or ‘respiratory condition’ refers to any one of several ailments that involve inflammation and affect a component of the respiratory system including the upper (including the nasal cavity, pharynx and larynx) and lower respiratory tract (including trachea, bronchi and lungs).

A symptom of respiratory disease may include cough, excess sputum production, a sense of breathlessness or chest tightness with audible wheeze. Exercise capacity may be quite limited. In asthma the FEV1.0 (forced expiratory volume in one second) as a percentage of that predicted nomographically based on weight, height and age, may be decreased as may the peak expiratory flow rate in a forced expiration. In COPD the FEV1.0 as a ratio of the FVC is typically reduced to less than 0.7. The impact of each of these conditions may also be measured by days of lost work/school, disturbed sleep, requirement for bronchodilator drugs, requirement for glucocorticoids including oral glucocorticoids.

The existence of, improvement in, treatment of or prevention of a respiratory disease may be determined by any clinically or biochemically relevant method of the subject or a biopsy therefrom. For example, a parameter measured may be the presence or degree of lung function, signs and symptoms of obstruction; exercise tolerance; night time awakenings; days lost to school or work; bronchodilator usage; inhaled corticosteroid (ICS) dose; oral (glucocorticoid) GC usage; need for other medications; need for medical treatment; hospital admission.

The terms “treatment” or “treating” of a subject includes the application or administration of a compound of the invention to a subject (or application or administration of a compound of the invention to a cell or tissue from a subject) with the purpose of delaying, slowing, stabilizing, curing, healing, alleviating, relieving, altering, remedying, less worsening, ameliorating, improving, or affecting the disease or condition, the symptom of the disease or condition, or the risk of (or susceptibility to) the disease or condition. The term “treating” refers to any indication of success in the treatment or amelioration of an injury, pathology or condition, including any objective or subjective parameter such as abatement; remission; lessening of the rate of worsening; lessening severity of the disease; stabilization, diminishing of symptoms or making the injury, pathology or condition more tolerable to the subject; slowing in the rate of degeneration or decline; making the final point of degeneration less debilitating; or improving a subject's physical or mental well-being.

A positive response to therapy may also be prevention or attenuation of worsening of respiratory symptoms, e.g. asthma symptoms (exacerbation), following a respiratory virus infection. This could be assessed by comparison of the mean change in disease score from baseline to end of study period based on Juniper Asthma Control Questionnaire (ACQ-6), and could also assess lower respiratory symptom score (LRSS—symptoms of chest tightness, wheeze, shortness or breath and cough) daily following infection/onset of cold symptoms. Change from baseline lung function (peak expiratory flow PEF) could also be assessed and a positive response to therapy could be a significant attenuation in reduced PEF. For example, a placebo treated group would show a significant reduction in morning PEF of 15% at the peak of exacerbation whilst the treatment group would show a non-significant reduction in PEF less than 15% change from baseline.

Although the invention finds application in humans, the invention is also useful for therapeutic veterinary purposes. The invention is useful for domestic or farm animals such as cattle, sheep, horses and poultry; for companion animals such as cats and dogs; and for zoo animals.

The composition according to the present invention is to be administered in an effective amount. The phrase ‘therapeutically effective amount’ or ‘effective amount’ generally refers to an amount of a TLR2 agonist, a pharmaceutically acceptable salt, polymorph or prodrug thereof of the present invention that (i) treats the particular disease, condition, or disorder, (ii) attenuates, ameliorates, or eliminates one or more symptoms of the particular disease, condition, or disorder, or (iii) delays the onset of one or more symptoms of the particular disease, condition, or disorder described herein. Undesirable effects, e.g. side effects, are sometimes manifested along with the desired therapeutic effect; hence, a practitioner balances the potential benefits against the potential risks in determining what is an appropriate “effective amount”.

In some embodiments, an effective amount for a human subject lies in the range of about 250 nmoles/kg body weight/dose to 0.005 nmoles/kg body weight/dose. Preferably, the range is about 250 nmoles/kg body weight/dose to 0.05 nmoles/kg body weight/dose. In some embodiments, the body weight/dose range is about 250 nmoles/kg, to 0.1 nmoles/kg, about 50 nmoles/kg to 0.1 nmoles/kg, about 5 nmoles/kg to 0.1 nmol/kg, about 2.5 nmoles/kg to 0.25 nmoles/kg, or about 0.5 nmoles/kg to 0.1 nmoles/kg body weight/dose. In some embodiments, the amount is at, or about, 250 nmoles, 50 nmoles, 5 nmoles, 2.5 nmoles, 0.5 nmoles, 0.25 nmoles, 0.1 nmoles or 0.05 nmoles/kg body weight/dose of the compound. Dosage regimes are adjusted to suit the exigencies of the situation and may be adjusted to produce the optimum therapeutic dose.

The exact amount required will vary from subject to subject, depending on the species, age and general condition of the subject, mode of administration and the like. Thus, it may not be possible to specify an exact “effective amount”. However, an appropriate “effective amount” in any individual case may be determined by one of ordinary skill in the art using only routine experimentation. In one aspect, the dose administered to a subject is any dose that reduces viral load.

The compounds comprising a TLR2 agonist as described herein may be in compositions formulated for administration to the URT only. Limitation to the URT may be achieved by an amount, particularly volume and composition of form ie. particle size, physical form whether dry powder or solution droplet, of composition that would otherwise be administered to the LRT or TRT. Alternatively, the compounds comprising a TLR2 agonist may be administered via a device that ensures retention in the URT only.

The compounds comprising a TLR2 agonist as described herein may be formulated for intranasal administration, including dry powder, sprays, mists, or aerosols. This may be particularly preferred for treatment of a respiratory infection.

There are a number of options that can be employed to limit delivery to the URT using intranasal delivery: (1) Ensuring the droplet/particle size is sufficiently large to prevent access into the LRT (>10 um); (2) For liquids limiting dose volume to minimise run-off/drainage; (3) Similarly administration with the head in an inverted position also minimises run-off/drainage; (4) Inclusion of a viscosity enhancer/mucoadhesive to promote retention in the nasal cavity and prevent run-off/drainage; (5) Use a nasal device that entirely eliminates the potential for LRT exposure e.g. the Optinose bi-directional delivery device. One or a combination of these methods can be applied.

Suitable formulations, wherein the carrier is a liquid, for administration, as for example, a nasal spray or as nasal drops, include aqueous or oily solutions of the active ingredient. Alternatively, the composition may be a dry powder and administered to the upper respiratory tract only as defined herein.

The selection of appropriate carriers depends upon the particular type of administration that is contemplated. For administration via the upper respiratory tract, e.g., the nasal mucosal surfaces, the compound can be formulated into a solution, e.g., water or isotonic saline, buffered or unbuffered, or as a suspension, for intranasal administration as drops or as a spray. Preferably, such solutions or suspensions are isotonic relative to nasal secretions and of about the same pH, ranging e.g., from about pH 4.0 to about pH 7.4 or, from pH 6.0 to pH 7.0. Buffers should be physiologically compatible and include, simply by way of example, phosphate buffers. For example, a representative nasal decongestant is described as being buffered to a pH of about 6.2 (Remington's, Id. at page 1445). Of course, the ordinary artisan can readily determine a suitable saline content and pH for an innocuous aqueous carrier for nasal and/or upper respiratory administration.

Other ingredients, such as art known preservatives, colorants, lubricating or viscous mineral or vegetable oils, perfumes, natural or synthetic plant extracts such as aromatic oils, and humectants and viscosity enhancers such as, e.g., glycerol, can also be included to provide additional viscosity, moisture retention and a pleasant texture and odour for the formulation. For nasal administration of solutions or suspensions according to the invention, various devices are available in the art for the generation of drops, droplets and sprays. For example, a compound or composition described herein can be administered into the nasal passages by means of a simple dropper (or pipet) that includes a glass, plastic or metal dispensing tube from which the contents are expelled drop by drop by means of air pressure provided by a manually powered pump, e.g., a flexible rubber bulb, attached to one end.

The tear secretions of the eye drain from the orbit into the nasal passages, thus, if desirable, a suitable pharmaceutically acceptable ophthalmic solution can be readily provided by the ordinary artisan as a carrier for the compound or composition described herein to be delivered and can be administered to the orbit of the eye in the form of eye drops to provide for both ophthalmic and intranasal administration.

In one embodiment, a premeasured unit dosage dispenser that includes a dropper or spray device containing a solution or suspension for delivery as drops or as a spray is prepared containing one or more doses of the drug to be administered. The invention also includes a kit containing one or more unit dehydrated doses of compound, together with any required salts and/or buffer agents, preservatives, colorants and the like, ready for preparation of a solution or suspension by the addition of a suitable amount of water. The water may be sterile or nonsterile, although sterile water is generally preferred.

The table below summarises various compounds referred to herein. Any of the compounds in the table below are contemplated as useful in any aspect of the invention.

| | Compound |
|---|---|
| Compound Structure | name |
| 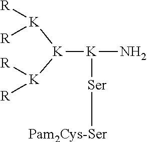 | INNA-001 |
| 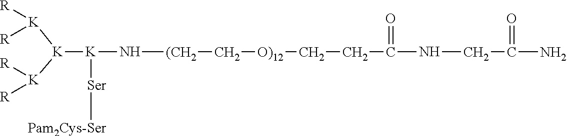 | INNA-002 |
| 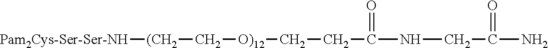 | INNA-003 |
| Pam2Cys-Ser-Ser-Lys-Lys-Lys-Lys | INNA-004 |
| Pam2Cys-Ser-Lys-Lys-Lys-Lys | INNA-005 |
| 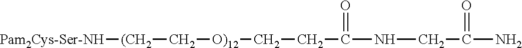 | INNA-006 (also shown herein as compound (1)) |
| 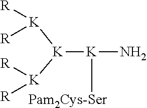 | INNA-007 |
| 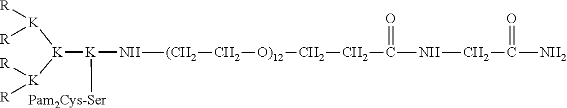 | INNA-008 |
| 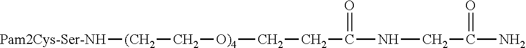 | INNA-009 |
| 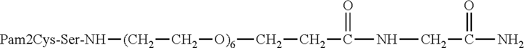 | INNA-010 |
| 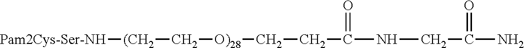 | INNA-011 (also shown herein as compound (5)) |
| 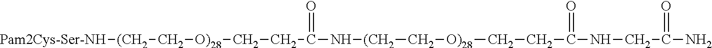 | INNA-012 (also shown herein as compound (6)) |
| 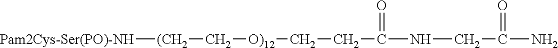 | INNA-013 (also shown herein as compound (4)) |
| 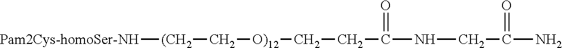 | INNA-014 (also shown herein as compound (3)) |
| 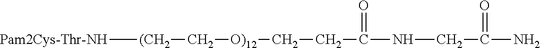 | INNA-015 (also shown herein as compound (2)) |
| 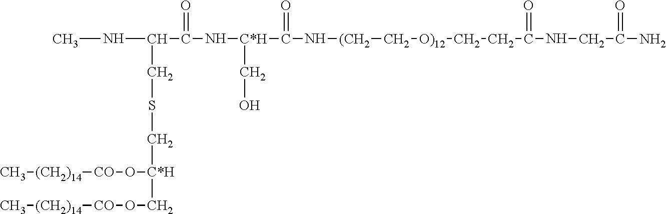 | N-Me-cysteine- INNA-006 (also shown herein as compound (9)) |
| 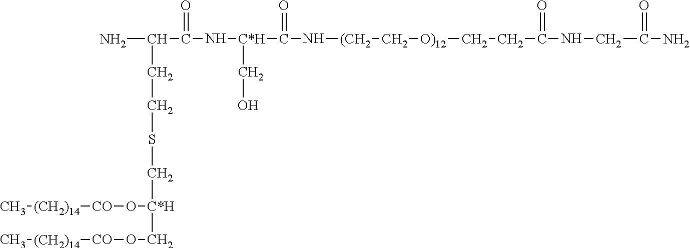 | L-Homo- cysteine-INNA- 006 (also shown herein as compound (10)) |
| 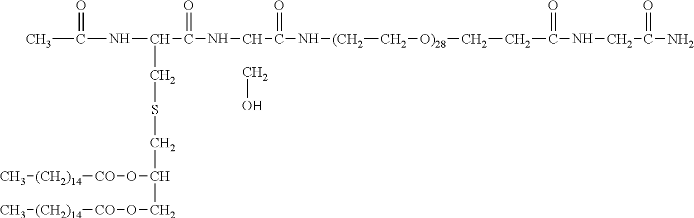 | N-acetyl-INNA- 011 (also shown herein as compound (11)) |
| 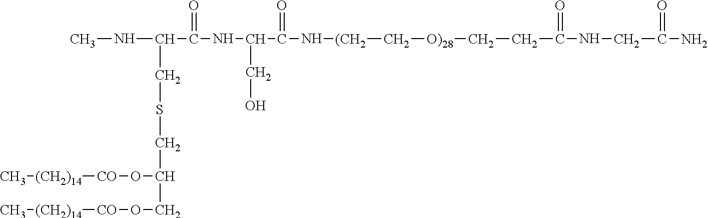 | N-methyl-INNA- 011 (also shown herein as compound (12)) |
| 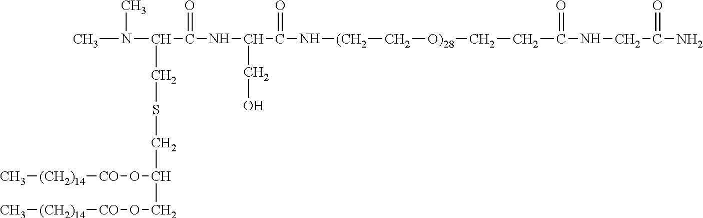 | N,N-dimethyl- INNA-011 (also shown herein as compound (13)) |
| 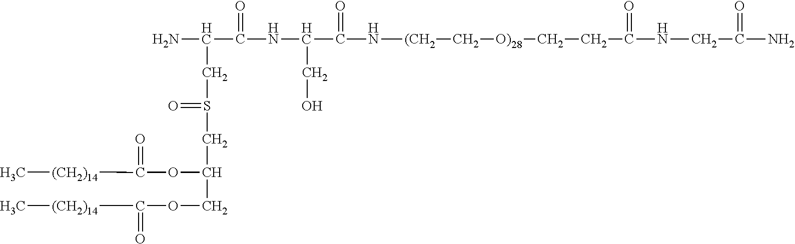 | Sulfoxide-INNA- 011 (also shown herein as compound (14)) |

### EXAMPLES

Natural exposure to influenza results in virus-containing airborne droplets being deposited in the URT where viral replication occurs with subsequent dissemination to the lower respiratory tract. Very little, if any, virus is transported to the lungs on first contact with virus. A procedure for mimicking this natural process has been successfully established in C57BL/6 mice and subsequently used to determine if compounds comprising TLR2 agonists can reduce replication and dissemination of influenza virus from the URT to the lungs.

### Example 1

When mice are inoculated intranasally, the inoculum can either be delivered to the total respiratory tract (TRT) or delivery can be restricted to the upper respiratory tract (URT) including the nasal passages. The destination of the inocula i.e. whether to the TRT or to the URT, depends upon the volume of inoculum administered and whether or not the mice are anaesthetised during the inoculation procedure.

To demonstrate that a volume of 10 μl restricts deposit of the inoculum to the URT, the inventors used influenza strain A/Puerto Rico/8 H1N1 (PR8) virus. When delivered intranasally and in a small volume to mice, growth of PR8 virus is restricted to the nasal passages of the URT but is prevented from moving to the lungs through the action of a salivary inhibitor that prevents progress of PR8 virus from nose to lungs.

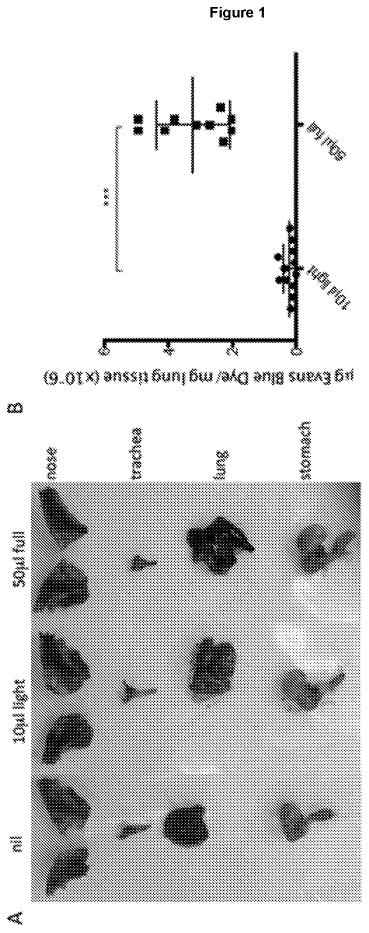

Five C57BL/6 mice were inoculated intranasally with 10 μl of PR8 virus while anaesthetised with isofluorane; the inventors found that virus grew in the nasal passages of all mice but no virus was detected in the lungs. In contrast, growth of PR8 virus was observed in nasal passages and in lungs of all mice that received virus delivered in a volume of 50 μl while anaesthetised (Table 1). This volume of inoculum is sufficiently large to lead to deposition of virus to nasal passages, trachea and lungs of animals i.e. TRT administration. These results were confirmed in an experiment where Evans Blue dye was instilled intranasally to anaesthetised mice in a volume of 10 μl or 50 μl after which the mice were killed dissected and the nasal turbinates, trachea, stomach and lungs were examined for the presence of dye (FIG. 1).

TABLE 1
Detection of infectious PR8 virus in the nasal passages
and lungs of mice challenged with 50 pfu of PR8 virus
delivered in either a 10 μL or 50 μL volume

| | Detection of infectious | Detection of infectious |
|---|---|---|
| | virus in nasal passages^{a} | virus in lungs^{a} |
| Inoculum: 50 pfu of | | |
| PR8 in 10 μl with | | |
| anaesthetic (URT) | | |
| Mouse 1 | + | − |
| Mouse 2 | + | − |
| Mouse 3 | + | − |
| Mouse 4 | + | − |
| Mouse 5 | + | − |
| Inoculum: 50 pfu of | | |
| PR8 in 50 μl with | | |
| anaesthetic (TRT) | | |
| Mouse 1 | + | + |
| Mouse 2 | + | + |
| Mouse 3 | + | + |
| Mouse 4 | + | + |
| Mouse 5 | + | + |

^{a}Tissues collected on day 4 post-challenge were homogenised and supernatants tested for the presence of infectious virus by plaguing on MDCK cell monolayers.
+ indicates viral growth and
− indicates no detectable virus.

Taken together, these results demonstrate that successful delivery to the URT can be achieved by administering inocula in a 10 μl volume to anaesthetised mice. This method of delivery has been used for all subsequent URT treatments including administration of various compounds comprising TLR2 agonists and influenza virus challenge studies described in the Examples below.

As used herein:

INNA-002 is also referred to as PEG-[Arg]4-Ser-Ser-Pam2Cys;

INNA-003 is also referred to as PEG-S—S-Pam2Cys (structure shown elsewhere herein); and

INNA-006 is also referred to as PEG-S-Pam2Cys (structure shown elsewhere herein).

### Example 2

Study 1A: Assessing the Progression of Udorn Virus to the Lower Respiratory Tract of Mice Following URT Challenge with 500 Pfu of Virus.

Aim: To determine a suitable time-point at which to harvest tissues from mice following challenge with 500 pfu of Udorn virus which is commensurate with a physiological infectious dose. The most suitable time-point will be the time at which virus can be detected in the lungs of all mice within a group.

Outline:
Eight groups of five C57BL/6 mice/group were challenged intranasally with 500 pfu of Udorn virus in a 10 μl volume using isoflurane anaesthesia.
Daily, from day 1 to day 8, a group of mice was killed and nasal turbinates, trachea and lungs removed, homogenised and supernatants frozen for subsequent determination of viral titre. Determination of plaque numbers were initially carried out on lung and nasal samples.

The experimental design is represented schematically below:

Results:

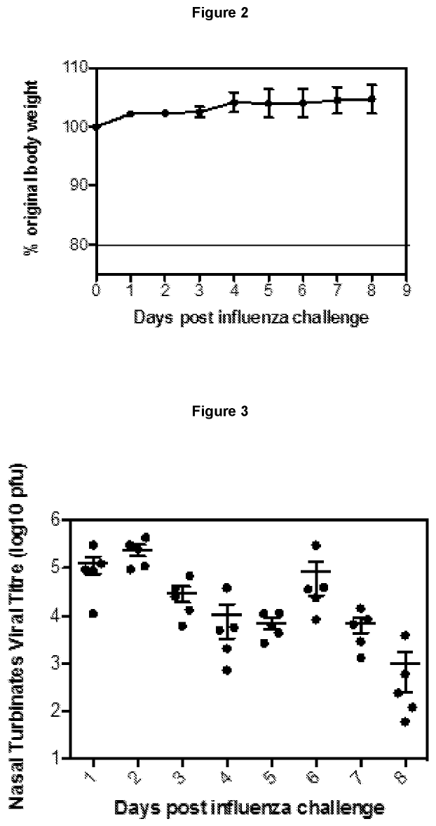

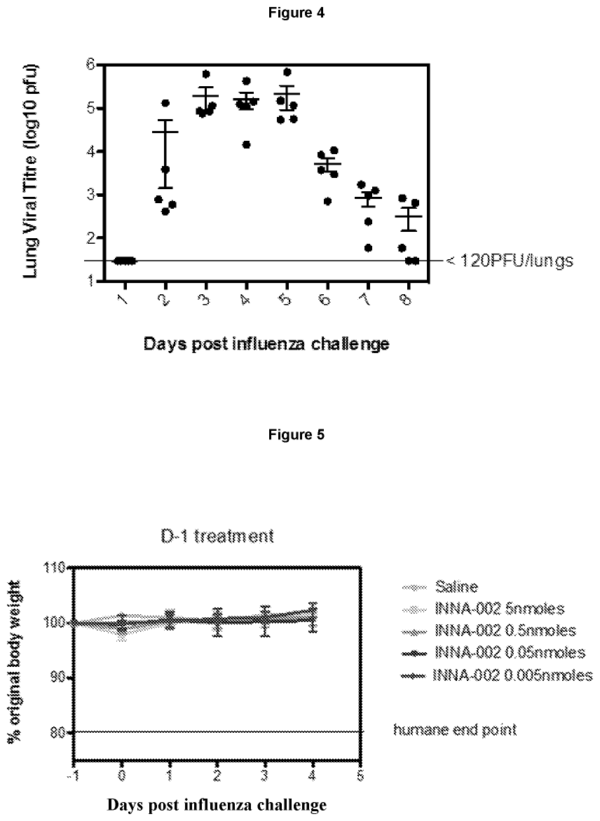

Although there was no significant change in weight observed in mice after challenge with 500 pfu of Udorn virus by the URT route (FIG. 2), virus could be detected in the nasal turbinates at titres which were up to 1,000 fold greater than the viral challenge dose (FIG. 3) confirming that under these conditions viral replication occurs at the site of inoculation. The progression of Udorn virus from the nose to the lungs was evident from day 2 post-challenge and reached a plateau on days 3-5 (FIG. 4) before viral titres waned from day 6 onwards. This study establishes a URT influenza challenge model in the mouse and identifies day 4 and 5 post-viral challenge as suitable time-points to harvest organs for viral assessment.

### Example 3

Study 1B: Effect of Pre-Treatment with Varying Doses of INNA-002 on URT Challenge with 500 Pfu Udorn Virus.

Aim: To determine the anti-viral effect of pre-treatment with varying doses of INNA-002 administered one day prior to challenge with Udorn virus.

Outline:
On day −1, mice (5 animals/group) received either saline, or 5 nmoles, 0.5 nmoles, 0.05 nmoles or 0.005 nmoles of INNA-002 administered intranasally in a volume of 10 μl while anaesthetised.
One day following administration of INNA-002 (day 0), mice were challenged intranasally with 500 pfu Udorn virus in a volume of 10 μl while anaesthetised.
Mice were killed 4 days after viral challenge with virus and nasal turbinates, trachea and lungs were removed, homogenised and supernatants frozen for subsequent determination of viral titres.

Results:

Relative to baseline weights, there was no significant weight loss apparent in C57/BL6 mice treated with INNA-002 in the dose range 0.005 nmoles-5 nmoles (FIG. 5).

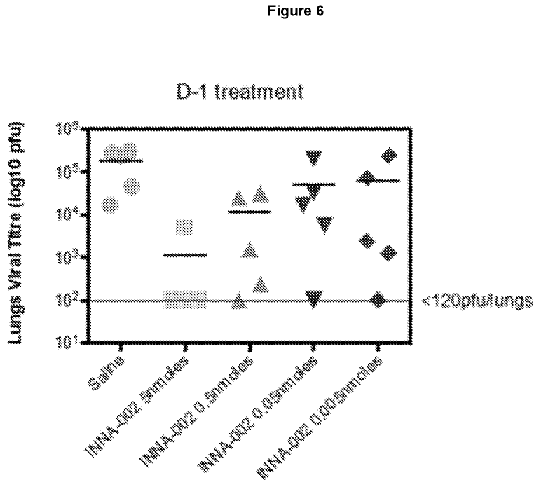

At a dose of 5 nmoles of INNA-002, progression of influenza virus to the lungs was inhibited in mice treated 1 day prior to viral challenge (FIG. 6). Mice receiving 0.5 nmoles of INNA-002 1 day before viral challenge displayed partial inhibition of influenza virus progression to the lungs. There was a dose-dependent antiviral-effect observed when INNA-002 was administered 1 day prior to challenge. The presence of influenza virus in the nasal turbinates of all mice at titres greater than the challenge dose (data not shown) confirms successful challenge using the URT influenza mouse model.

### Example 4

Study 1C: Assessing the Effect of Pre-Treatment with Varying Doses of INNA-003 on URT Challenge with Udorn Virus

Aim: To determine the effect of pre-treatment with varying doses of INNA-003 one day prior to URT challenge with Udorn virus.

Outline:
On day −1, mice (5 animals/group) received either saline, 5 nmoles, 0.5 nmoles, 0.05 nmoles or 0.005 nmoles of INNA-003 administered intranasally in 10 μl volume while anaesthetised.
One day following administration of INNA-003 (day 0), mice were challenged intranasally with 500 pfu of Udorn virus in 10 μl while anaesthetised.
Mice were killed on 4 days after challenge and nasal turbinates, trachea and lungs were removed, homogenised and supernatants frozen for subsequent determination of viral titres.

Results:

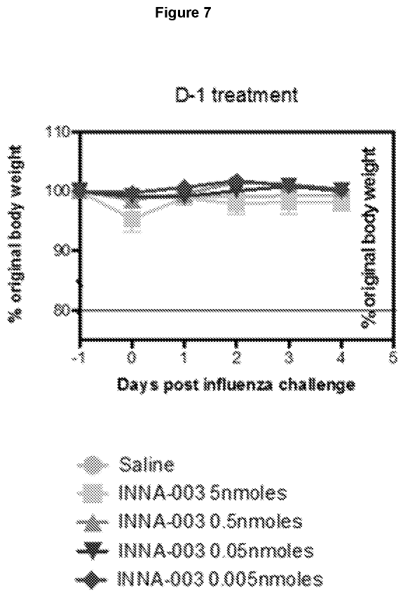

Weight loss observed in C57BL/6 mice treated with INNA-003 in the dose range 0.005 nmoles-5 nmoles are shown in FIG. 7. The highest weight loss (4.67%±2.12%) was apparent in a single animal receiving 5 nmoles (FIG. 7).

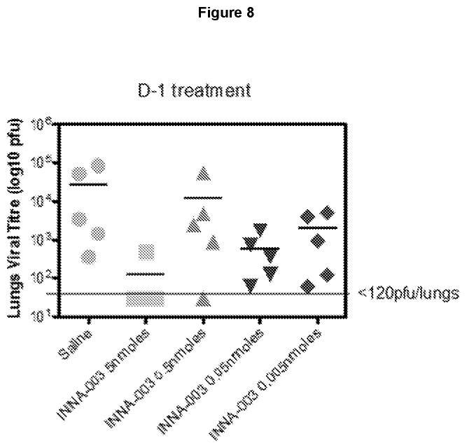

As shown in FIG. 8, a dose of 5 nmoles of INNA-003 inhibited progression of influenza virus to lungs when administered one day prior to viral challenge. At lower doses of INNA-003 sporadic inhibition of virus was observed. For all mice, the presence of viral titres considerably higher than the inoculating dose in the nasal turbinates (data not shown) confirms that viral challenge to the URT was successful.

### Example 5

Study 2A: Assessing the Effect of Pre-Treatment with Different Doses of INNA-003 or INNA-006 on the Outcome of URT Challenge with Udorn Virus

Aim: To determine the anti-viral effect of URT pre-treatment with various doses of INNA-003 or INNA-006.

Outline:
On day −1 mice (5 animals/group) received either saline, 5 nmoles, 0.1 nmoles or 0.005 nmoles of INNA-003 or INNA-006, administered intranasally in 10 μl after being anaesthetized with isoflurane.
One day following administration of the TLR2 agonists (day 0), mice were challenged intranasally with 500 pfu of Udorn virus in a volume of 10 μl after being anaesthetized with isoflurane.
Mice were killed on day 5 and nasal turbinates trachea and lungs were removed, homogenised and frozen for subsequent analyses.

The experimental design is summarised in the schematic below:

Results:

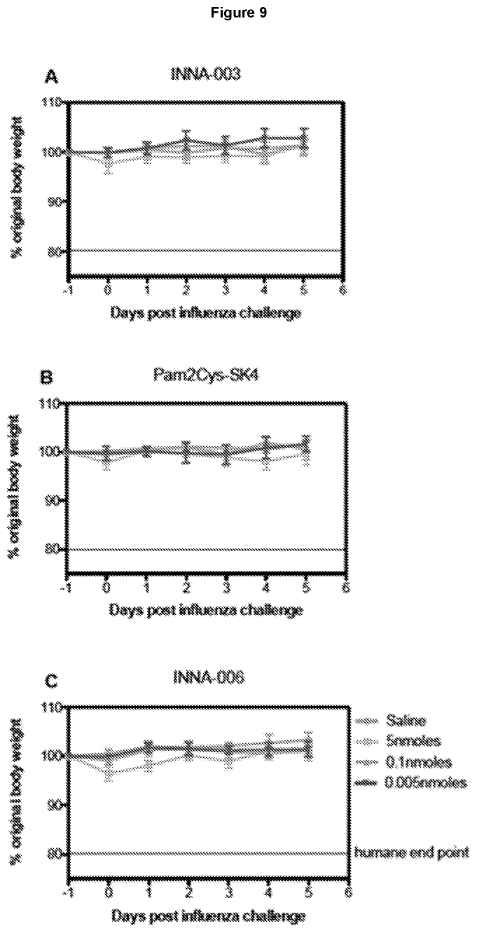

Relative to baseline weights, there was little or no weight loss in C57BL/6 mice treated with INNA-003, Pam2Cys-SK4 or INNA-006 in the dose range 0.005 nmoles-5 nmoles (FIG. 9).

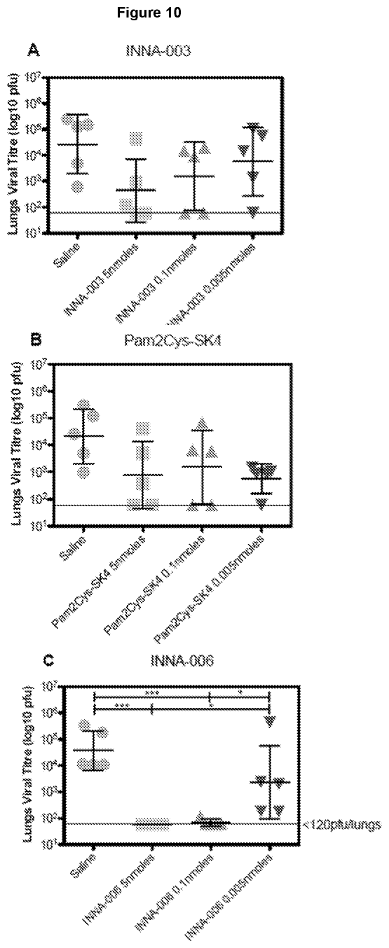

At doses of 5 nmoles and 0.1 nmoles of INNA-006, progression of influenza virus to the lungs was significantly inhibited in mice treated 1 day prior to viral challenge (FIG. 10C). Mice receiving 0.005, 0.1 or 5 nmoles of INNA-003 or Pam2Cys-SK4, or 0.005 nmoles of INNA-006 displayed partial inhibition of influenza virus progression to the lungs (FIGS. 10A and C). Influenza virus titres in the nasal turbinates of all mice were ˜100-fold higher than the titre of the challenge dose confirming successful viral challenge using the URT model (data not shown).

### Example 6

Study 2B: Assessing Effects of Multiple Doses of Candidate TLR2 Agonists when Administered to the URT

Outline: The treatment protocol for this study is summarised in Table 2. Groups of 5 female C57BL/6 mice received either 3 treatment doses or a single treatment dose of INNA-003 or INNA-006 at 2 different concentrations. All treatments were administered to the URT of anaesthetised mice in 10 μl volumes. Mice were weighed daily and one day after the final treatment, animals were killed and blood, nasal turbinates, trachea and lungs harvested, homogenised and assayed for cytokine content.

TABLE 2
Inoculation protocol to assess the effect of multiple doses of INNA-003
and INNA-006 measured by weight loss and cytokine profiles.

| | | | Day(s) | Day of |
|---|---|---|---|---|
| | | Number | inoculum | Tissue |
| Group | Treatment | of doses | administered | Harvest |
| 1 | Saline | 3 | Days 0, 2, 4 | Day 5 |
| 2 | INNA-003 0.5 nmoles | 3 | Days 0, 2, 4 | Day 5 |
| 3 | INNA-003 0.05 nmoles | 3 | Days 0, 2, 4 | Day 5 |
| 4 | INNA-006 0.5 nmoles | 3 | Days 0, 2, 4 | Day 5 |
| 5 | INNA-006 0.05 nmoles | 3 | Days 0, 2, 4 | Day 5 |
| 6 | Saline | 1 | Day 4 | Day 5 |
| 7 | INNA-003 0.5 nmoles | 1 | Day 4 | Day 5 |
| 8 | INNA-003 0.05 nmoles | 1 | Day 4 | Day 5 |
| 9 | INNA-006 0.5 nmoles | 1 | Day 4 | Day 5 |
| 10 | INNA-006 0.05 nmoles | 1 | Day 4 | Day 5 |

The experimental design is summarised in the schematic below:

Results:

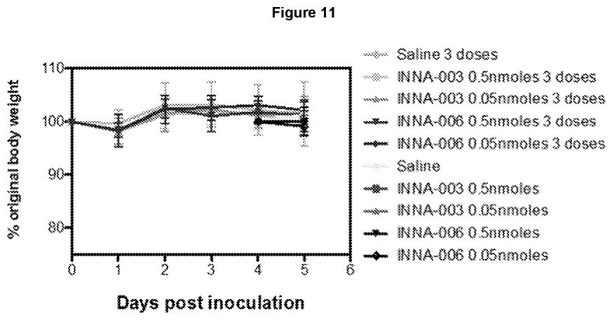

Relative to baseline, there was no significant weight loss apparent in C57BL/6 mice following URT treatment with either a single dose or 3 consecutive doses of INNA-003 or INNA-006 at concentrations of 0.05 nmoles or 0.5 nmoles (FIG. 11). The greatest weight loss, 1.89%±1.15%, was observed following the first of 3 repeated doses of 0.5 nmoles INNA-003.

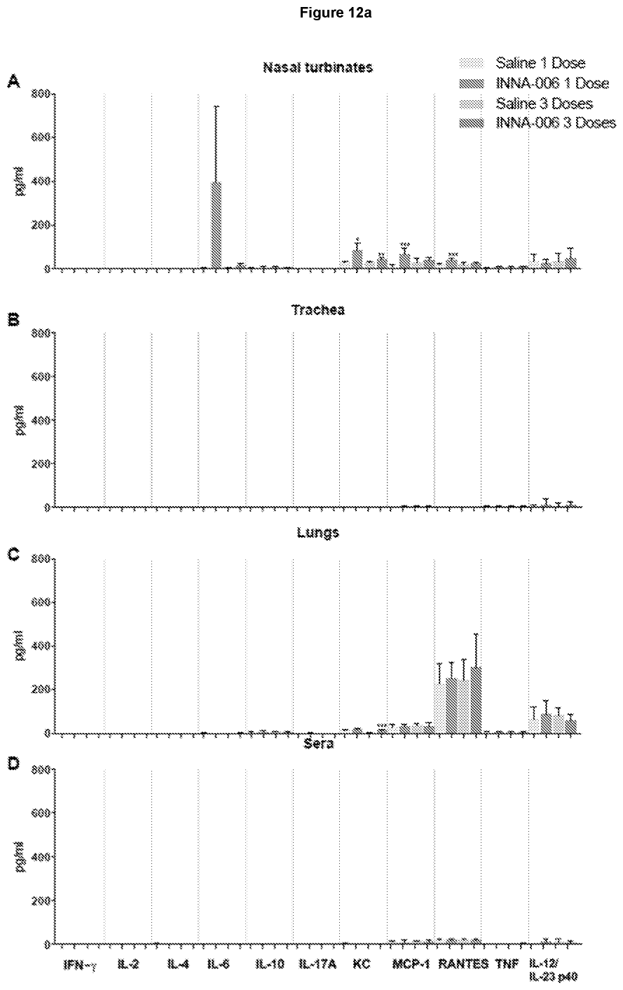

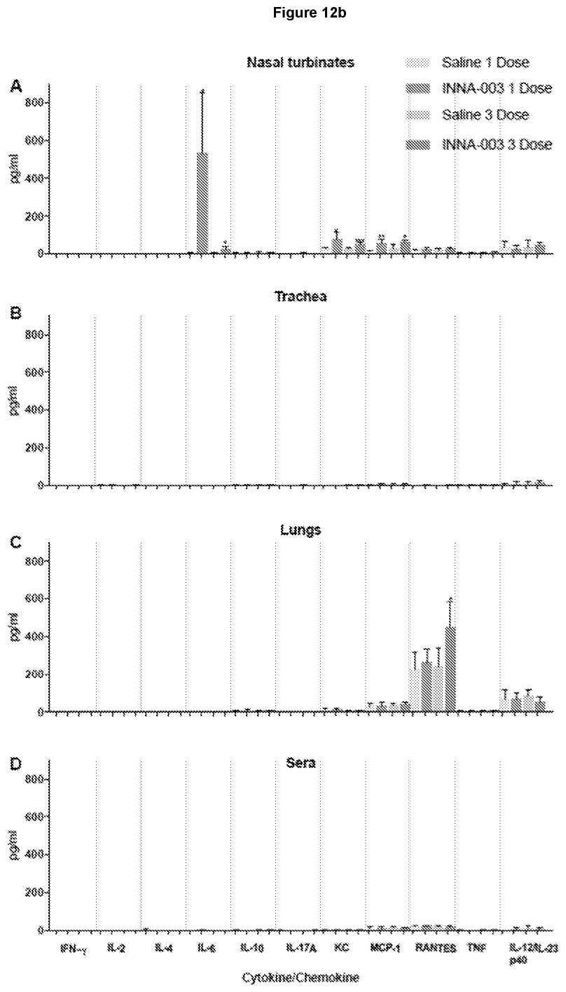

FIGS. 12a, B and 13 shows the cytokine/chemokine profiles that were detected in the nasal turbinates, lungs, trachea and sera of mice following either a single or 3 repeat doses (0.5 nmoles or 0.05 nmoles) of INNA-003 or INNA-006. The cytokine/chemokine profiles detected in lungs, trachea and sera showed no discernible differences when compared between groups of animals treated with either single or triple dose regimes. Differences in the cytokine/chemokine profiles were observed in nasal turbinates (FIGS. 12A and B) with an increase in proinflammatory cytokines and chemokines including IL-6, KC & MCP-1. These increases were detected in a dose-dependent manner for both INNA-003 and INNA-006 when compared to saline control treatment (FIGS. 12A and B). Mice treated with a single dose of INNA-006 (0.5 nmoles) showed increased RANTES secretion in the nasal turbinates when compared to animals that received INNA-003.

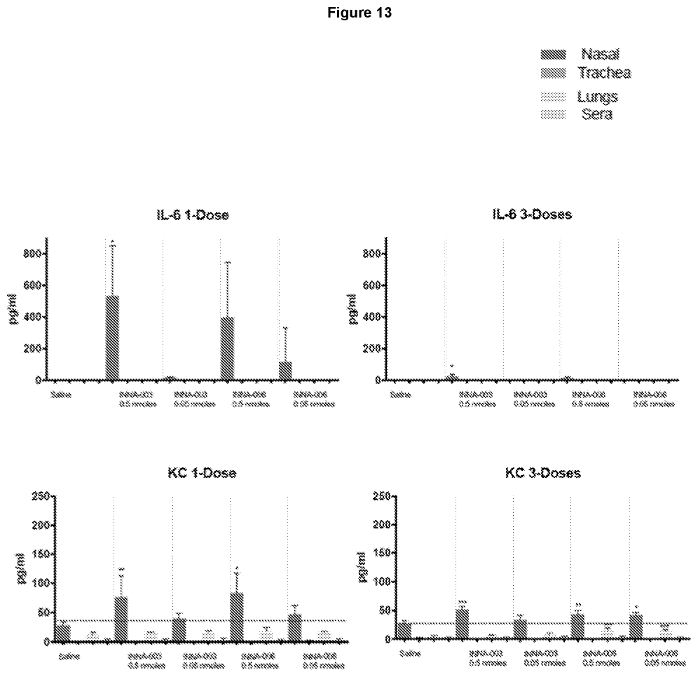

Mice that were treated with 3 repeat doses of either INNA-006 or INNA-003 showed a marked decrease in cytokine/chemokine levels in the nasal turbinates, lungs and sera when compared to mice that had received a single dose of either compound (FIG. 13).

Statistically significant increases of IL-6, and KC levels were apparent in the nasal turbinates of mice treated with agonist compared with those treated with saline but these increases were significantly less in animals receiving multiple treatments (FIG. 13). For example, the level of IL-6 was approximately 25-fold lower in the nasal turbinates of mice treated with 3 doses (0.5 nmoles) of either INNA-003 or INNA-006 (˜22 pg/ml and 15 pg/ml respectively) when compared to levels detected in mice that received a single dose of the same compound. In mice receiving a single dose (0.5 nmoles) of agonist, there was an approximately 125-fold (530 pg/ml: 4 pg/ml) increase in IL-6 levels with 0.5 nmoles INNA-003 and a 98-fold increase (˜395 pg/ml: ˜4 pg/ml) with INNA-006.

After a single treatment with 0.5 nmole of INNA-003 or INNA-006, there was a 6-fold (˜54 pg/ml: 9 pg/ml) and a 7-fold (˜66 pgml: ˜9 pg/ml) increase respectively of MCP-1 in the nasal turbinates. Again, these levels were reduced in the groups receiving 3 doses of agonist. A 2-fold increase compared to the relative saline control group in the 0.5 nmoles INNA-003 treatment group only (˜60 pg/ml: ˜27 pg/ml) was also apparent.

A statistically significant difference was detected in the levels of KC present in nasal turbinates of mice that received a single treatment of TLR2 agonist viz, a 2-fold increase for animals receiving 0.5 nmoles INNA-003 (˜76 pg/ml: 27 pg/ml) and INNA-006 (˜83 pgml: ˜27 pg/ml) with no statistically significant difference observed between the 3 dose treatment groups receiving 3 doses of TLR2 agonist or saline.

There was no significance of KC in the lungs of mice treated with either INNA-003 or INNA-006 after a single dose compared to the saline control (FIG. 13). However a statistical significance was apparent in the levels of KC in lungs of mice treated with the 3-dose regime (FIG. 13); both 0.5 and 0.05 nmole doses of INNA-006 produced a moderate 2 fold increase of KC secretion in lungs compared to INNA-003 (˜14 pg/ml:7.5 pg/ml) and a 3 fold increase compared to the saline control treatments (˜14 pg/ml: ˜5 pg/ml).

There was a statistically significant, 2-fold reduction, of KC levels in the lung when treated with 3 doses of INNA-003 when compared to INNA-006 treatment (˜7.5 pg/ml: ˜16.3 pg/ml)).

### Example 7

Study 2C: Effect of Multiple Doses of INNA-003 or INNA-006 Followed by Challenge with Influenza Virus on Body Weight, Lung Virus Titres and Cytokine Profiles.

Aim: To confirm that multiple dosing of INNA-003 or INNA-006 is still effective at inhibiting viral dissemination to the lungs of treated animals.

Outline: The treatment and challenge protocol for this study is summarised in Table 2. Groups of 5 C57BL/6 mice were treated with 3 doses of either saline, INNA-003 or INNA-006. One day after the third dose mice were challenged with influenza virus and 5 days later lungs were collected for determination of viral titres. Levels of selected cytokines were also measured in nasal turbinates and lungs of these animals.

The experimental design is summarised in the schematic below:

Results:

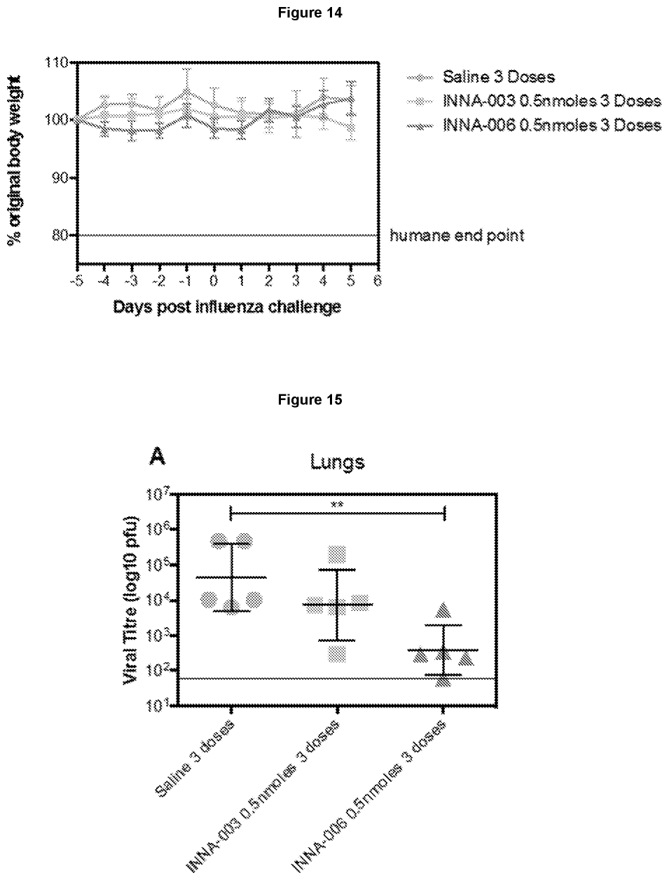

Relative to baseline, there was no statistically significant weight loss in mice treated with 0.5 nmoles of INNA-003 or INNA-006 (FIG. 14). The greatest weight loss was 1.87%±1.33% which was observed at Day −3 post influenza virus challenge in the treatment group receiving INNA-006.

Following three repeated doses of 0.5 nmoles of INNA-006, progression of influenza virus to the lungs was significantly inhibited compared to animals which received saline. Mice receiving three doses of 0.5 nmoles of INNA-003 displayed partial inhibition of influenza virus progression to the lungs (FIG. 15). The amounts of influenza virus in the nasal turbinates of mice was also determined (data not shown) to demonstrate that all animals had increased levels of virus in the nasal turbinates indicating successful introduction and subsequent replication of virus.

Cytokines/chemokine levels were measured in the nasal turbinates and lungs of mice 5-days post-influenza challenge. Compared to mice which received saline we observed statistically significant increases of IL-10 with 3 doses of 0.5 nmoles of INNA-006 (2 fold ˜17 pg/ml: ˜9 pg/ml) and RANTES with 3 doses of 0.5 nmoles of INNA-006 (1.9 fold ˜141 pg/ml: ˜73 pg/ml) and INNA-003 (1.7 fold ˜130 pg/ml: ˜73 pg/ml). These increases were only associated with the nasal turbinates i.e. the site of agonist administration. No such significant increases were detected in the lungs of treated animals.

### Example 8

Methods & Materials

Synthesis of INNA-002

INNA-002 (PEG-[Arg]4-Ser-Ser-Pam2Cys; described in WO2016037240) was synthesized by and purchased from AusPep Pty. Ltd. PO Box 566, Tullamarine, Victoria 3043, Australia.

Synthesis of INNA-003 and INNA-006

Reagents: Solid phase support: TentaGel S RAM resin (substitution factor 0.24 mmol/g; Rapp Polymere, Tübingen, Germany). Amino acid derivatives: Fmoc-Gly-OH, Fmoc-Ser(tBu)-OH, Fmoc-homo-Ser(tBu)-OH, Fmoc-Ser(PO(OBzl)OH)—OH, Fmoc-Thr(tBu)-OH, Fmoc-NH-(PEG)₃-COOH, Fmoc-NH-(PEG)₅-COOH, Fmoc-NH-(PEG)₁₁-COOH, Fmoc-NH-(PEG)₂₇-COOH from Merck (Darmstadt, Germany).

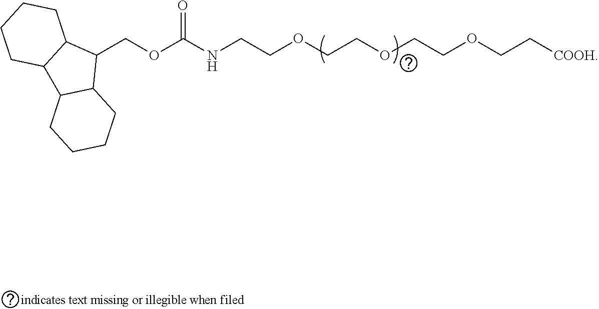

NB use of Merck catalogue number 851024 gives rise to the structures shown below as “INNA-003” (which may also be referred to herein as Pam2Cys-SS-PEG) and “INNA-006” (which may also be referred to herein as Pam2Cys-S-PEG).

Acylation: A 4-fold molar excess of Fmoc amino acid, O-benzotriazole-N,N,N′,N′-tetramethyl-uroniumhexafluorophosphate (HBTU) and a 6-fold molar excess of diisopropylethylamine (DIPEA) are used in all acylation steps. All acylation reactions are carried out for 60 minutes and completion of reaction confirmed by trinitrobenezene sulfonic acid (TNBSA) test. Removal of the Fmoc protective group from α-amino groups is achieved by exposing the solid phase support to 2.5% diazabicyclo[5.4.0]undec-7-ene (DBU; Sigma, Steinheim, Germany) for 2×5 minutes. dimethylformamide (DMF; Auspep, Melbourne, Australia) is used to wash the solid phase support between each acylation and de-protection step. The coupling of Fmoc-NH-(PEG)₁₁-COOH (Merck, Bayswater, Australia) is carried out in the same way as coupling amino acids.

NB. Glycine is first coupled to the TentaGel S RAM solid phase support followed by Fmoc-NH-(PEG)₁₁-COOH.

Peptide Quantitation

Quantitation of peptide-based materials was determined by amino acid analysis performed in vacuo by hydrolysis of samples at 110° C. in sealed glass vials in the presence of 6N HCl containing 0.1% phenol. Derivatisation of amino acids was then carried out using Waters AccQTag reagents according to the manufacturer's instructions followed by analysis on a Waters Acquity UPLC System (Waters Millipore) using an AccQTag ultra column (2.1 mm×100 mm; Waters Millipore).

Preparation of INNA-003 and INNA-006

In the case of INNA-003 two serine residues are coupled seriatim following addition of the PEG moiety and in the case of INNA-006 a single serine is incorporated following addition of the PEG moiety.

Lipidation (Addition of Pam2Cys).

Synthesis of S-(2,3-dihydroxypropyl)cysteine: Triethylamine (6 g, 8.2 ml, 58 mmoles) is added to L-cysteine hydrochloride (3 g, 19 mmole) and 3-bromo-propan-1,2-diol (4.2 g, 2.36 ml, 27 mmole) in water and the homogeneous solution kept at room temperature for 3 days. The solution is reduced in vacuo at 40° C. to a white residue which is then precipitated with acetone (300 ml) and the precipitate isolated by centrifugation. The precipitate is washed with acetone twice more and dried to yield S-(2,3-dihydroxypropyl)cysteine as a white amorphous powder.

Synthesis of N-Fluorenylmethoxycarbonyl-S-(2,3-dihydroxypropyl)-cysteine (Fmoc-Dhc-OH): S-(2,3-dihydroxypropyl) cysteine (2.45 g, 12.6 mmole) is dissolved in 9% sodium carbonate (20 ml). A solution of fluorenylmethoxycarbonyl-N-hydroxysuccinimide (3.45 g, 10.5 mmole) in acetonitrile (20 ml) is then added and the mixture stirred for 2 h, diluted with water (240 ml) and extracted with diethyl ether (25 ml×3). The aqueous phase is acidified to pH 2 with concentrated hydrochloric acid and then extracted with ethyl acetate (70 ml×3). The extract is washed with water (50 ml×2) and saturated sodium chloride solution (50 ml×2). The extract is dried over anhydrous sodium sulphate and evaporated to dryness. The final product is obtained by applying high vacuum to remove residual solvent.

Coupling of Fmoc-Dhc-OH to resin-bound peptide: Fmoc-Dhc-OH (100 mg, 0.24 mmole) is activated in DCM and DMF (1:1, v/v, 3 mL) with HOBt (36 mg, 0.24 mmole) and DICI (37 uL, 0.24 mmole) at 0° C. for 5 min. The mixture is then added to a vessel containing the resin-bound peptide (0.04 mmole, 0.25 g amino-peptide resin). After shaking for 2 h the solution is removed by filtration on a glass sinter funnel (porosity 3) and the resin washed with DCM and DMF (3×30 mL each). The reaction is monitored for completion using the TNBSA test. If necessary a double coupling is performed.

Palmitoylation of the two hydroxyl groups of the Fmoc-Dhc-peptide resin: Palmitic acid (204 mg, 0.8 mmole), DIPCDI (154 uL, 1 mmole) and DMAP (9.76 mg, 0.08_mmole) are dissolved in 2 mL of DCM and 1 mL of DMF. The resin-bound Fmoc-Dhc-peptide_resin (0.04 mmole, 0.25 g) is suspended in this solution and shaken for 16 h at room temperature. The solution is removed by filtration and the resin then washed with DCM and_DMF thoroughly to remove any residue of urea. The removal of the Fmoc group is accomplished with 2.5% DBU (2×5 min).

Cleavage of peptide from the solid support: Reagent B (93% TFA, 5% water and 2% triisopropylsilane) for two hours. NB the peptide will not precipitate in chilled ether. Most of the TFA must be removed and then the residue is dissolved in 50% acetonitrile and purified immediately or freeze-dried.

Purification and Characterisation of INNA-003 and INNA-006

Following cleavage from the solid support, INNA-003 and INNA-006 were purified by reversed-phase high-performance liquid chromatography using a C4 VYDAC column (10 mm×250 mm; Alltech, NSW, Australia) installed in a Waters HPLC system (Waters Millipore, Milford, Mass., USA). Identity of the target materials were determined by mass spectrometry and the purified material was then characterised by analytical HPLC using a VYDAC C8 column (4.6 mm×250 mm) and found to be greater than 95%. Mass analysis was carried out using an Agilent 1100 Series LC/MSD ion-trap mass spectrometer (Agilent, Palo Alto, Calif., USA).

Preparation of compound (2) or Pam2Cys-Thr-PEG, a single threonine is incorporated following the addition of the PEG11 moiety. The addition of Pam2Cys (lipidation) was carried out as described above.

Preparation of compound (3) or Pam2Cys-homoSer-PEG, a single homo-serine is incorporated following the addition of the PEG11 moiety. The addition of Pam2Cys (lipidation) was carried out as described above.

Preparation of compound (4) or Pam2Cys-phosphoSer-PEG, a single phosphoserine is incorporated following the addition of the PEG11 moiety. The addition of Pam2Cys (lipidation) was carried out as described above.

Preparation of Pam2Cys-Ser-PEG3, PEG3 moiety instead of PEG11 was coupled following the coupling of the first amino acid glycine. After the coupling of a single serine residue the addition of Pam2Cys (lipidation) was carried out as described above.

Preparation Pam2Cys-Ser-PEG5, PEG5 instead of PEG11 moiety was coupled following the coupling of the first amino acid glycine. After the coupling of a single serine residue the addition of Pam2Cys (lipidation) was carried out as described above.

Preparation of compound (5), PEG27 instead of PEG11 moiety was coupled following the coupling of the first amino acid glycine. After the coupling of a single serine residue the addition of Pam2Cys (lipidation) was carried out as described above

Preparation of compound (6), PEG27 moiety was coupled sequentially twice following the coupling of the first amino acid glycine. After the coupling of a single serine residue the addition of Pam2Cys (lipidation) was carried out as described above.

Preparation of compound (2a), two threonines are incorporated following the addition of the PEG11 moiety. The addition of Pam2Cys (lipidation) is carried out as described above.

Preparation of compound (3a), a two homo-serines are incorporated following the addition of the PEG11 moiety. The addition of Pam2Cys (lipidation) is carried out as described above.

Preparation of compound (4a), two phosphoserines are incorporated following the addition of the PEG11 moiety. The addition of Pam2Cys (lipidation) is carried out as described above.

Preparation of compound (5a), PEG27 instead of PEG11 moiety was coupled following the coupling of the first amino acid glycine. After the coupling of two serine residues the addition of Pam2Cys (lipidation) is carried out as described above

Preparation of compound (6a), PEG27 moiety was coupled sequentially twice following the coupling of the first amino acid glycine. After the coupling of two serine residues the addition of Pam2Cys (lipidation) is carried out as described above.

Experimental Animals

Groups of either 5 or 10 male or female, 6-8 week old C57BL/6 mice were used for all studies. After administration of saline, INNA-00x or viral challenge, mice were monitored daily for weight changes, and behavioural or physical changes as stipulated in animal ethics approval #1513638.

URT Administration of Innavac Compounds

Mice were anaesthetized by isoflurane inhalation and saline or various doses of the Innavac compounds, diluted in saline, were administered intranasally in a total volume of 10 μl using a pipettor. For the multi-treatment experiments mice received 3 doses of INNA-003 or INNA-006 every second day over a 5-day period.

Preparation of Influenza Virus

A/Udorn/307/72 (H3N2) influenza virus was propagated in the allantoic cavity of 10 day-old embryonated hens'eggs. Eggs were inoculated with approximately 10³ pfu of virus in 0.1 ml of saline. After 2 days incubation at 35° C. the eggs were chilled at 4° C. and allantoic fluid harvested and clarified by centrifugation. Viral infectivity titre (pfu/mL) was determined by plaque assay as described below and aliquots of the allantoic fluid were stored at −80° C. until used.

URT Virus Challenge

Mice were anaesthetised with isofluorane and inoculated intranasally with 500 pfu of Udorn virus in 10 μl of saline, using a pipettor. On day 4 or 5 post-challenge the nasal turbinates, trachea and lung were harvested to assess viral loads.

Extraction and Preparation of Nasal Turbinates, Trachea and Lung Homogenates

Mice were killed by CO₂ asphyxiation 24 hours after their last scheduled treatment or 5 days post-influenza challenge. Nasal turbinates, trachea and lungs from each mouse were collected in 1.5 mL of RPMI-1640 medium with antibiotics (100 μg/mL penicillin, 180 μg/mL streptomycin and 24 μg/mL gentamicin) and kept on ice until processed. Tissues were homogenised using a tissue homogeniser and the resulting organ homogenates then centrifuged at 2,000 rpm for 5 min to remove cell debris. Supernatants were collected and stored at −80° C. for subsequent measurements.

Assessment of Viral Titres

Titres of infectious Udorn virus were determined by plaque assay on confluent monolayers of Madin Darby canine kidney (MDCK) cells. Six-well tissue culture plates were seeded with 1.2×10⁶ MDCK cells per well in 3 ml of RF10 (RPMI-1640 medium supplemented with 10% (v/v) heat inactivated FCS, 260 μg/mL glutamine, 200 μg/mL sodium pyruvate, 100 μg/mL penicillin, 180 μg/mL streptomycin and 24 μg/mL gentamicin). After overnight incubation at 37° C. in 5% CO₂ confluent monolayers were washed with RPMI-1640 medium. Test supernatants serially diluted in RPMI-1640 with antibiotics, were added to duplicate wells of monolayers. After incubation at 37° C. in 5% CO₂ for 45 min, monolayers were overlaid with 3 mL of agarose overlay medium prewarmed to 45° C. The overlay consisted of 9 mg/MI agarose and 2 μg/mL trypsin-TPCK treated in Leibovitz L15 medium pH6.8 with glutamine supplanted with 800 μM HEPES, 0.028% w/v NaHCO₃, 100 μg/mL penicillin and 180 μg/mL streptomycin. Plates were incubated for 3 days at 37° C. in 5% CO₂ and virus-mediated cell lysis then counted as plaques on the cell layer by visual inspection without staining. The total organ viral titres (plaque forming units, PFU) for individual animals were then calculated.

Determination of Cytokine Levels in Nasal Turbinates, Trachea, Lungs and Sera

IFN-γ, IL-2, IL-4, TNF, IL-10, IL-6, KC, MCP-1, RANTES, IL-12/IL-23p40 and IL-17A present in nasal turbinates, trachea, lung homogenates and serum samples were measured using a BD Cytometric Bead Array (CBA) Flex Kit according to the manufacturer's instructions with the exception that a total of 0.15 μl of each capture bead suspension and 0.15 μl of each PE-detection reagent was used in each 50 μl sample. Samples were analysed using a Bection Dickinson FACSCanto II flow cytometer and the data analysed using FCAP Array multiplex software.

Statistical Analyses

A one-way analysis of variance (ANOVA) with Tukey comparison of all column tests was used. A two-way ANOVA with Bonferroni's test was used to compare the same treatment groups in the single and 3 repeat dose regimes. A p-value 0.0322 was considered statistically significant. Statistical analyses were performed using Graph Pad Prism, version 7.0.

### Example 9

Assessing the Effect of Pre-Treatment with INNA-011 on the Outcome of Challenge with Udorn Virus, when INNA-011 is Administered to the URT

The effect on viral replication of URT treatment with 5 nmoles of INNA-011 7 days prior to influenza challenge with 500 pfu of Udorn virus was investigated. On day 0, mice (10 animals/group) received either saline or 5 nmoles of INNA-011 administered intranasally to the URT in a volume of 10 μl while anaesthetised. On day 7 following administration of INNA-011, mice were challenged intranasally with 500 pfu Udorn virus in a volume of 10 μl while anaesthetised. Mice were killed 5 days after challenge with virus and nasal turbinates, trachea and lungs were removed, homogenised and supernatants frozen for subsequent determination of viral titres.

Little or no weight loss was apparent in C57BL/6 mice treated with INNA-011 using a dose of 5 nmoles (data not shown).

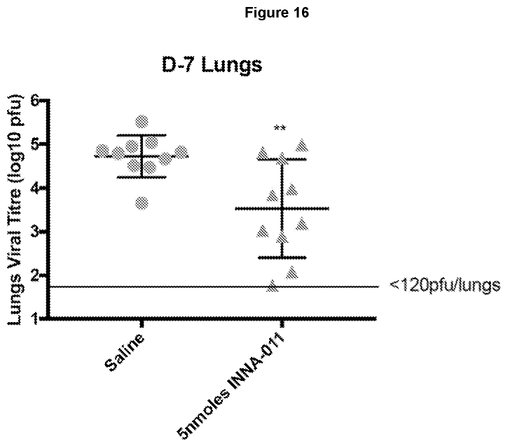

Mice treated on Day-7 with 5 nmoles of INNA-011 to the URT only were able to significantly inhibit progression of influenza virus to the lungs when compared to saline controls (FIG. 16).

### Example 10

Assessing the Effect of One Day Pre-Treatment with INNA-011 on the Outcome of Challenge with Udorn Virus, when INNA-011 is Administered to the URT

The effect on viral replication of URT treatment with 5, 1 or 0.25 nmoles of INNA-011 one day prior to influenza challenge with 500 pfu of Udorn virus was investigated.

On day 0, mice (5 or 7 animals/group) received either saline/PBS or 5, 1 or 0.25 nmoles of INNA-011 administered intranasally to the URT in a volume of 10 μl while anaesthetised. One day following administration of INNA-011, mice were challenged intranasally with 500 pfu Udorn virus in a volume of 10 μl while anaesthetised. Mice were killed 5 days after challenge with virus and nasal turbinates, trachea and lungs were removed, homogenised and supernatants frozen for subsequent determination of viral titres.

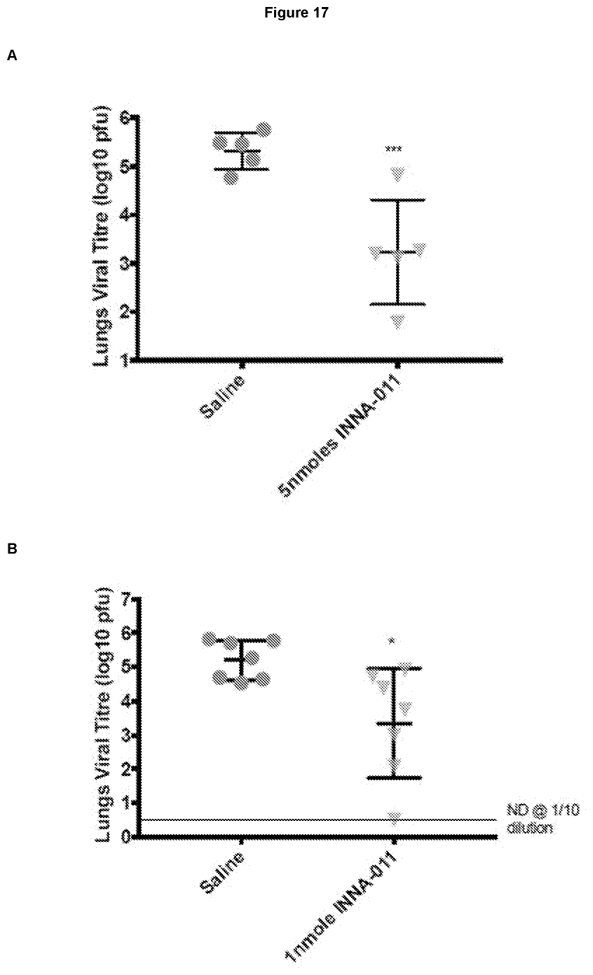

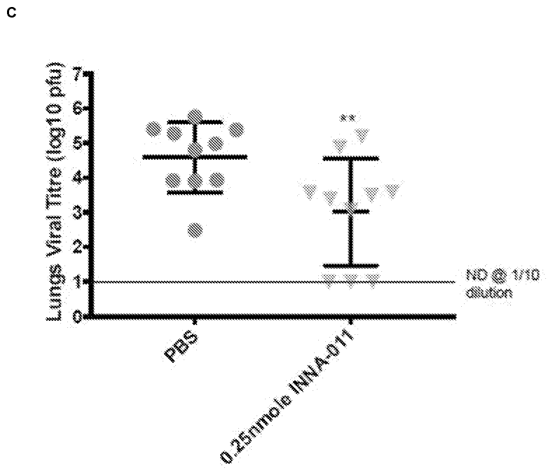

Mice treated with 5, 1 or 0.25 nmoles INNA-011 to the URT only were able to significantly inhibit progression of influenza virus to the lungs when compared to saline controls (FIG. 17A-C).

### Example 11—Synthesis of the R and S Isomers of INNA-006 and INNA-011, Around the Chiral Centre of 2,3-Bis(palmitoyloxy)propyl

R and S isomers of the Pam2 moiety of Fmoc S-2,3-di(palmitoyloxypropyl)-cysteine (Fmoc-Dpc) were purchased from Bachem Inc. which were then used to synthesise the R and S-Pam2 isomers of INNA-006 and INNA-011 as described in Example 8 above.

The synthesised compounds were characterised using HPLC, mass spectrometry and amino acid analysis (AAA). The stereochemistry of the compounds was determined by measuring their optical activity using standard methods in the art.

### Example 12—Synthesis of INNA-006 and INNA-011 Analogues

### General Synthesis Protocol for Assembly of INNA-011 Analogues:

Reagents: The solid phase support, TentaGel S RAM resin (substitution factor 0.24 mmol/g; Rapp Polymere, Tubingen, Germany) was used throughout with glycine coupled to the solid phase support as the first residue (see below). The amino acid derivatives: Fmoc-Gly-OH, Fmoc-Ser(tBu)-OH and Fmoc-N-Me-Cys(Trt)-OH were obtained from Auspep or Merck. Fmoc-NH-(PEG)₂₇-COOH (88 atoms) was from Merck (Cat #851033, Darmstadt, Germany). Borane-dimethylamine-complex (abbreviated as ABC) was from Sigma-Aldrich (cat #180238-5G). 16% (w/v) formaldehyde (methanol free) was from Pierce (cat #28906) or alternatively a 16% methanol-free solution of paraformaldehyde was obtained from from Electron Microscopy Sciences (cat #15710). The sources of other materials are indicated in the Appendices.

Acylation: A 4-fold molar excess of Fmoc amino acid, O-benzotriazole-N,N,N′,N′-tetramethyl-uroniumhexafluorophosphate (HBTU) and a 6-fold molar excess of diisopropylethylamine (DIPEA) were used in all acylation steps. All acylation reactions were carried out for 60 minutes or as indicated in each individual step and completion of reaction confirmed by trinitrobenezene sulfonic acid (TNBSA) test. Removal of the Fmoc protective group from α-amino groups was achieved by exposing the solid phase support to 2.5% diazabicyclo[5.4.0]undec-7-ene (DBU; Sigma, Steinheim, Germany) for 2×5 minutes. Dimethylformamide (DMF; Auspep, Melbourne, Australia) was used to wash the solid phase support between each acylation and de-protection steps. The coupling of Fmoc-NH-(PEG)₂₇-COOH was carried out in the same way as the coupling of amino acids.

### Synthesis of Fmoc-PEG27-Gly-Resin

Fmoc-Gly (297 mg, 1 mmole in 4 ml of DMF) was added as the first amino acid to the solid support (0.5 g, 0.125 mmole), followed by coupling of Fmoc-NH-PEG27-COOH (300 mg, 0.194 mmole; 0.237 mmole of HBTU, 0.26 mmole of HOBT and 0.36 mmole of DIPEA in 2 ml of DMF) for 2 hrs. After washing, equal portions (0.0425 mmole) of the solid phase support, to which was attached Fmoc-NH-PEG₂₇-Gly, was used to assemble the four different analogues as described below.

### Addition of Pam2Cys

Synthesis of S-(2,3-dihydroxypropyl)cysteine: Triethylamine (6 g, 8.2 ml, 58 mmoles) was added to L-cysteine hydrochloride (3 g, 19 mmole) and 3-bromo-propan-1,2-diol (4.2 g, 2.36 ml, 27 mmole) in water and the solution held at room temperature for 3 days. The solution was reduced in vacuo at 40° C. to a white residue which was then precipitated with acetone (300 ml) and the precipitate isolated by centrifugation. The precipitate was washed twice with acetone and dried to yield S-(2,3-dihydroxypropyl)cysteine as a white amorphous powder.

Synthesis of N-Fluorenylmethoxycarbonyl-S-(2,3-dihydroxypropyl)-cysteine (Fmoc-Dhc-OH): S-(2,3-dihydroxypropyl) cysteine (2.45 g, 12.6 mmole) was dissolved in 9% sodium carbonate (20 ml). A solution of fluorenylmethoxycarbonyl-N-hydroxysuccinimide (3.45 g, 10.5 mmole) in acetonitrile (20 ml) was then added and the mixture stirred for 2 h, diluted with water (240 ml) and extracted with diethyl ether (25 ml×3). The aqueous phase was acidified to pH2 with concentrated hydrochloric acid and then extracted with ethyl acetate (70 ml×3). The extract was washed with water (50 ml×2) and saturated sodium chloride solution (50 ml×2). The extract was dried over anhydrous sodium sulphate and evaporated to dryness. The final product was obtained by applying high vacuum to remove residual solvent.

Coupling of Fmoc-Dhc-OH to resin-bound peptide: Fmoc-Dhc-OH (100 mg, 0.24 mmole) was activated in DCM and DMF (1:1, v/v, 3 mL) with HOBt (36 mg, 0.24 mmole) and DICI (37 uL, 0.24 mmole) at 0° C. for 5 min. The mixture was then added to a vessel containing the resin-bound peptide (0.04 mmole, 0.25 g amino-peptide resin). After shaking for 2 h the solution was removed by filtration on a glass sinter funnel (porosity 3) and the resin washed with DCM and DMF (3×30 mL). The reaction was monitored for completion using the TNBSA test. If necessary a double coupling was performed.

Palmitoylation of the two hydroxyl groups of the Fmoc-Dhc-peptide resin: Palmitic acid (204 mg, 0.8 mmole), DIPCDI (154 uL, 1 mmole) and DMAP (9.76 mg, 0.08 mmole) were dissolved in 2 mL of DCM and 1 mL of DMF. The resin-bound Fmoc-Dhc-peptide_resin (0.04 mmole, 0.25 g) was suspended in this solution and shaken for 16 h at room_temperature. The supernatant was removed by filtration and the resin thoroughly washed with DCM and_DMF to remove any residue of urea. The removal of the Fmoc group was accomplished using 2.5% DBU (2×5 min).

Cleavage of peptide from the solid support: The solid support bearing the assembled lipopeptide was exposed to reagent B (93% TFA, 5% water and 2% triisopropylsilane) for two hours. NB the peptide will not precipitate in chilled ether. Most of the TFA must be removed and the residue is then dissolved in 50% acetonitrile and purified immediately or freeze-dried.

Purification and characterisation: Following cleavage from the solid support, each of the analogues were purified by reversed-phase HPLC using a C4 Vydac column (10 mm×250 mm; Alltech, NSW, Australia) installed in a Waters HPLC system (Waters Millipore, Milford, Mass., USA). Identification of the target materials were determined by mass spectrometry and the purified material was then characterised by analytical HPLC using a VYDAC C8 column (4.6 mm×250 mm) and found to be greater than 95%. Mass analysis was carried out using an Agilent 1100 Series LC/MSD ion-trap mass spectrometer (Agilent, Palo Alto, Calif., USA).

### Synthesis of N-acetyl-INNA-011

Synthesis of N-acetyl-INNA-011 was carried out by acetylation of the amino group of cysteine residue of Pam2Cys with the peptide still attached to the solid phase as set out in the below schematic. Cleavage from the solid support and purification yielded the final product.

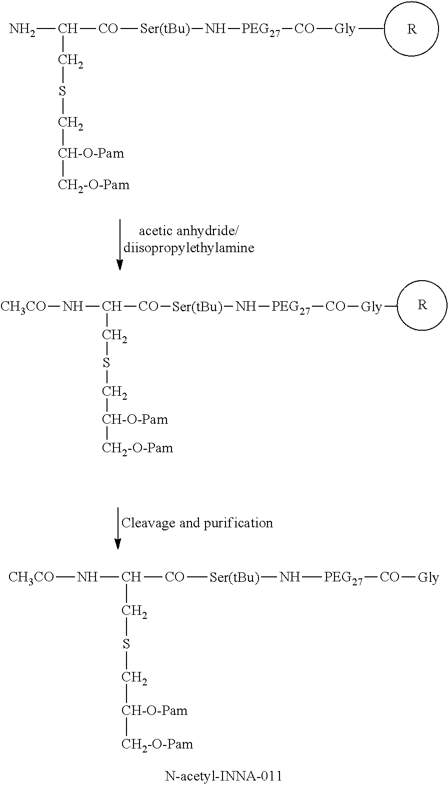

Following the removal of Fmoc group, 220 mg (0.5 mmole) of N-Fluorenylmethoxycarbonyl-S-(2,3-dihydroxypropyl)-cysteine (Fmoc-Dhc-OH), 67 mg of HOBt and 200 μl of DICI in 2 ml of DMF were added to one of the portions of Fmoc-NH-PEG₂₇-Gly-resin made as described above and the reaction was held at RT for 3 hrs. The two hydroxyl groups were palmitoylated as described above. The Fmoc group was removed and the exposed α-amino group acetylated by incubation with 1 ml of acetylanhydride and 100 μl of DIPEA for 30 mins. The peptide was cleaved from the solid support and purified as described below. The qualitative analysis of the purified final product was carried out by amino acid analysis (AAA) and LC-MS analysis.

### Synthesis of N-methyl-INNA-011 and L-Homo-cysteine-INNA-006

The synthesis of N-methyl-INNA-011 was carried out using a protocol for synthesis of Pam2Cys-containing peptides as described in the below schematic. Briefly, Fmoc-N-methyl-Cys(Trt)-OH was coupled to Ser(tBu)-NH-PEG27-Gly which was attached to the solid support. The primary α-amino group was then blocked with a tert-butyloxycarbonyl (Boc) group. The subsequent removal of the protecting trityl group and alkylation of the sulfhydryl group with 1-bromo-2,3-propanediol followed by palmitoylation of the two vicinal hydroxyl groups yielded N-methyl-INNA-011.

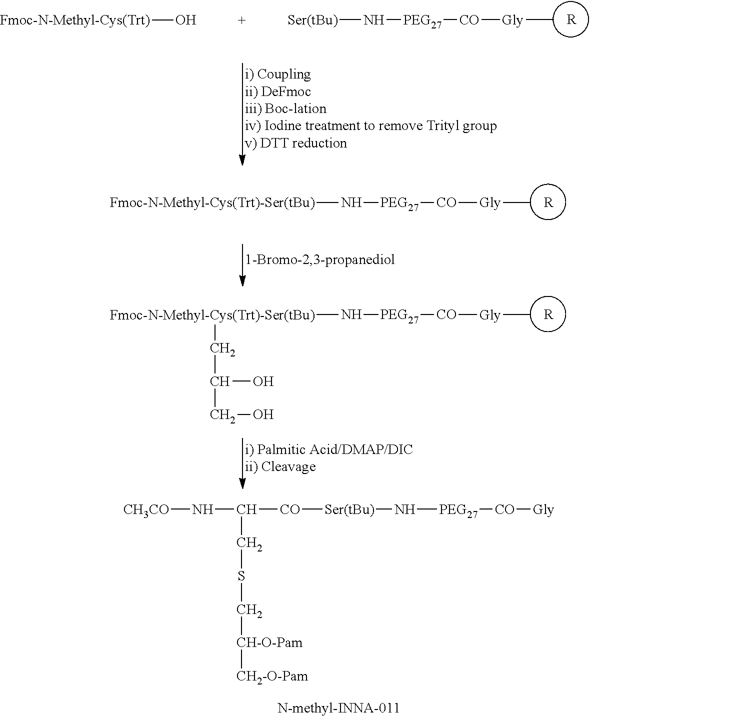

To one of the portions of Fmoc-NH-PEG₂₇-COOH was added Fmoc-N-methyl-Cys(Trt)-OH (102 mg, 0.17 mmole, 65 mg of HBTU, 23 mg of HOBt and 46 μl of DIPEA in 2 ml of DMF) for 2 hr. The Fmoc-group was removed and the exposed primary amino group then blocked with di-tert-butyl-dicarbonate (1 ml plus 100 μl of DIPEA) overnight. The Trt group was removed by immersing the peptide resin in an iodine solution (254 mg of iodine in 8 ml of DMF) pre-chilled in a salt-ice bath for 5 mins. The whole peptide-resin and iodine suspension was kept on ice-salt for 1 hr. The peptide resin was washed in a glass sinter funnel with saturated ascorbic acid until the the peptide resin was colourless and then further washed with DMF. The peptide was then treated with dithiolthreitol (154 mg in 1.5 ml of DMF plus 0.5 ml of 0.2M phosphate buffer at pH8) for 1 hr at RT. Following thorough washing with DMF the exposed sulfhydryl group was alkylated by suspending the peptide resin in 200 μl of 1-bromo-2,2-propanediol and 10 μl of DIPEA in 1 ml of DMF for 3 hrs. The final palmitoylation of the two hydroxy groups was carried out as described above._The peptide was cleaved from the solid support and purified as described below. The qualitative analysis of the purified final product was carried out by AAA and LC-MS analysis.

L-Homo-cysteine-INNA-006 was synthesised by using Fmoc-homoCys(Trt)-OH and Ser(tBu)-NH-PEG11-Gly in the above method.

### Synthesis of N,N-dimethyl-INNA-011

N,N-dimethyl-INNA-011 was prepared by reductive methylation of the primary alpha-amino group of Pam2Cys present in INNA-011 in the presence of formaldehyde and borane-dimethylamine-complex (ABC) as set out in the below schematic.

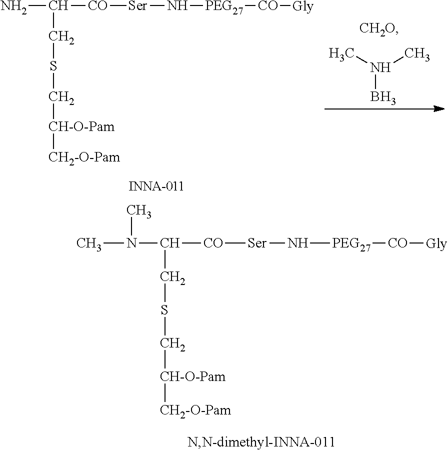

A solution of borane-dimethylamine complex, ABC (220 mg in 1 ml of water, 3.3 M) was freshly made. To 5 mg of INNA-011 was added 1 ml of 3.3M solution of ABC followed by addition, 3 times, of 187 μl 16% formaldehyde solution (NB. this reaction is strongly exothermic). The reaction was left at RT for 3 hrs. An additional 187 μl of 16% formaldehyde solution was then added and incubated at RT for 1 hr. LC-MS analysis indicated that reaction was complete. The product was isolated by semipreparative HPLC. Qualitative analysis of the purified product was carried out by LC-MS and amino acid analysis.

### Synthesis of Sulfoxide-INNA-011

Sulfoxide-INNA-011 was prepared by oxidising INNA-011 in the presence of hydrogen peroxide as set out in the below schematic. Briefly, INNA-011 was dissolved in water and to it was added an equal volume of 30% hydrogen peroxide. The reaction was held at RT overnight (16 hrs). The majority of the final product was sulfoxide-INNA-011 with a very small amount of sulfone-INNA-011. These two oxidation products were easily separated by HPLC.

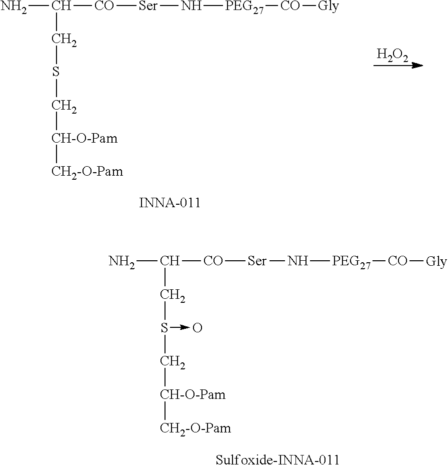

To 10 mg of INNA-011 dissolved in 300 μl of water was added 300 μl of 30% hydrogen peroxide (Sigma-Aldrich) and the reaction was held at RT overnight. LC-MS analysis indicated a completed reaction and the final product was isolated by semipreparative HPLC. Quality analysis of the purified product was carried out by AAA and LC-MS. It will be understood that the invention disclosed and defined in this specification extends to all alternative combinations of two or more of the individual features mentioned or evident from the text or drawings. All of these different combinations constitute various alternative aspects of the invention.

1. A method of treating or preventing a respiratory condition associated with an infectious agent in an individual, the method comprising administering a compound comprising a Toll-like receptor 2 (TLR2) agonist to the upper respiratory tract of the individual, thereby treating or preventing a respiratory condition associated with an infectious agent in the individual.

2. Use of a compound comprising a TLR2 agonist in the preparation of a medicament for treating or preventing a respiratory condition associated with an infectious agent in an individual, wherein the medicament is adapted for administration to the upper respiratory tract.

3. Use of a compound comprising a TLR2 agonist for the treatment or prevention of a respiratory condition associated with an infectious agent in an individual, wherein the compound is adapted for administration to the upper respiratory tract.

4. The method or use according to any one of claims 1 to 3, further comprising a step of identifying a subject having a respiratory condition associated with an infectious agent.

5. A method of inhibiting or reducing the amount of an infectious agent in the lung of an individual, the method comprising administering a compound comprising a TLR2 agonist to the upper respiratory tract of the individual, thereby inhibiting or reducing the amount of an infectious agent in the lung of an individual.

6. Use of a compound comprising a TLR2 agonist in the preparation of a medicament for inhibiting or reducing the amount of an infectious agent in the lung of an individual, wherein the medicament is adapted for administration to the upper respiratory tract.

7. Use of a compound comprising a TLR2 agonist for inhibiting or reducing the amount of an infectious agent in the lung of an individual, wherein the compound comprising a TLR2 agonist is adapted for administration to the upper respiratory tract.

8. A method of inhibiting, delaying or reducing the progression of an infectious agent from the upper respiratory tract to the lungs of an individual, the method comprising administering a compound comprising a TLR2 agonist to the upper respiratory tract of the individual, thereby inhibiting, delaying or reducing the progression of the infectious agent from the upper respiratory tract to the lungs of the individual.

9. Use of a compound comprising a TLR2 agonist in the preparation of a medicament for inhibiting, delaying or reducing the progression of an infectious agent from the upper respiratory tract to the lungs of an individual, wherein the medicament is adapted for administration to the upper respiratory tract.

10. Use of a compound comprising a TLR2 agonist for inhibiting, delaying or reducing the progression of an infectious agent from the upper respiratory tract to the lungs of an individual, wherein the compound comprising a TLR2 agonist is adapted for administration to the upper respiratory tract.

11. A method or use according to any one of claims 1 to 10, wherein the compound comprising a TLR2 agonist is not administered to the lower respiratory tract or is not administered to both the upper and lower respiratory tract (i.e. is not administered to the total respiratory tract).

12. A method or use according to any one of claims 1 to 11, wherein the compound comprising a TLR2 agonist is administered to the nose and nasal passages.

13. A method or use according to any one of claims 1 to 11, wherein the compound comprising a TLR2 agonist is administered to the nose, nasal passages and paranasal sinuses.

14. A method or use according to any one of claims 1 to 11, wherein the compound comprising a TLR2 agonist is administered to the nose, nasal passages, paranasal sinuses and the pharynx.

15. A method or use according to any one of claims 1 to 11, wherein the compound comprising a TLR2 agonist is administered to the nose, nasal passages, paranasal sinuses, the pharynx and the portion of the larynx above the vocal folds (cords).

16. A method or use according to claim 15, wherein the compound is administered intranasally.

17. A method or use according to claim 16, wherein the compound is administered as a nasal spray or drops.

18. A method or use according to claim 17, wherein the droplet or particle size is sufficiently large to prevent access into the lower respiratory tract.

19. A method or use according to claim 17, wherein the droplet or particle size is greater than 10 μm.

20. A method or use according to any one of claims 1 to 15, wherein the compound is administered as a liquid in an amount that avoids drainage into the lower respiratory tract.

21. A method or use according to any one of claims 1 to 15, wherein the compound is administered to the individual with a head in an inverted position to avoid drainage into the lower respiratory tract.

22. A method or use according to any one of claims 1 to 15, wherein the compound is administered with a viscosity enhancer or mucoadhesive to promote retention in the nasal cavity.

23. A method or use according to any one of claims 1 to 15, wherein the compound is administered using a nasal device that entirely eliminates the potential for lower respiratory tract exposure, preferably the device is the OptiNose bi-directional delivery device.

24. The method or use according to any one of claims 1 to 23, wherein there is no significant increase of one or more of pro-inflammatory cytokines IL-10, IL-6, KC, MCP-1, RANTES, IL-12 or TNF-α in the lungs or lower respiratory tract when compared to pro-inflammatory cytokine levels of the upper respiratory tract.

25. A method or use according to any one of claims 1 to 24, wherein method or use does not comprise administering agonists of TLRs other than TLR2 homodimers or heterodimers.

26. A method or use according to any one of claims 1 to 25, wherein the compound is administered in a composition that further comprises a pharmaceutically acceptable carrier, diluent or excipient.

27. A method or use according to claim 26, wherein composition consists of a compound comprising a TLR2 agonist and a pharmaceutically acceptable carrier, diluent or excipient.

28. A method or use according to any one of claims 1 to 27, wherein the infectious agent is a virus or bacteria.

29. A method or use according to any one of claims 1 to 28, wherein the infectious agent is a virus.

30. A method or use according to claim 29, wherein the virus is influenza.

31. A method or use according to claim 30, wherein the method further comprises a step of identifying a subject having an influenza infection.

32. A method or use according to any one of claims 1 to 31, wherein the TLR2 agonist comprises a lipid, a peptidoglycan, a lipoprotein or a lipopolysaccharide.

33. A method or use according to any one of claims 1 to 32, wherein the TLR2 agonist comprises palmitoyl, myristoyl, stearoyl, lauroyl, octanoyl, or decanoyl.

34. A method or use according to any one of claims 1 to 33, wherein the TLR2 agonist is selected from the group consisting of: Pam2Cys, Pam3Cys, Ste2Cys, Lau2Cys, and Oct2Cys.

35. A method or use according to claim 34, wherein the TLR2 agonist is Pam2Cys.

36. A method or use according to claim 34, wherein the TLR2 agonist is not Pam3Cys.

37. A method or use according to any one of claims 1 to 36, wherein the solubility of the TLR2 agonist is increased by a solubilising agent.

38. A method or use according to any one of claims 1 to 37, wherein the compound comprises a TLR2 agonist and a solubilising agent.

39. A method or use according to claim 37 or 38, wherein the TLR2 agonist and solubilising agent are linked.

40. A method or use according to any one of claims 37 to 39, wherein the solubilising agent comprises or consists of a positively or negatively charged group.

41. A method or use according to claim 40, wherein the charged group is a branched or linear peptide.

42. A method or use according to claim 40 or 41, wherein the positively charged group comprises at least one positively charged amino acid, preferably an arginine or lysine residue.

43. A method or use according to claim 40 or 41, wherein the negatively charged group comprises at least one negatively charged amino acid, preferably a glutamate or aspartate.

44. A method or use according to any one of claims 41 to 43, wherein the branched or linear peptide is R4, H4, H8 or E8.

45. A method or use according to any one of claims 37 to 44, wherein the solubilising agent comprises polyethyleneglycol (PEG) or R4.

46. A method or use according to claim 44, wherein the solubilising agent comprises polyethyleneglycol (PEG) and R4.

47. A method or use according to claim 46, wherein the PEG is PEG₁₁.

48. A method or use according to any one of claims 1 to 31, wherein the compound comprising a TLR2 agonist comprises the structure:
A-Y-B
wherein A comprises or consists of: 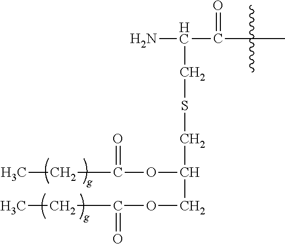
wherein each g is independently 10, 11, 12, 13, 14, 15, 16, 17 or 18;
Y is 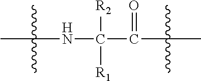
wherein R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₂ are not both H;
and
B comprises or consists of Polyethylene Glycol (PEG),
or a pharmaceutically acceptable salt or prodrug thereof.

49. A method or use according to any one of claims 1 to 31, wherein the compound is of formula (I): 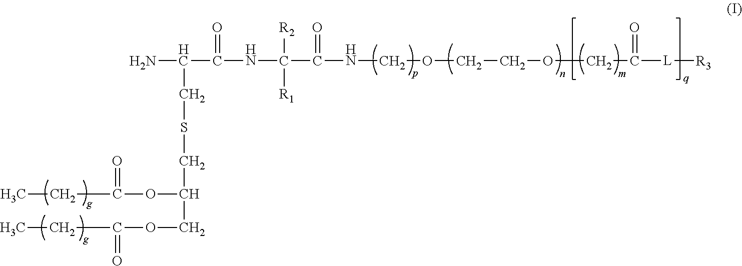
wherein
n is 3 to 100;
m is 1, 2, 3 or 4;
each g is independently 10, 11, 12, 13, 14, 15, 16, 17 or 18;
p is 2, 3 or 4;
q is null or 1;
R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₂ are not both H;
wherein when q=1, R₃ is —NH₂ or —OH;
wherein when q=0, R₃ is H;
L is null or consists of 1 to 10 units, wherein each unit is a natural alpha amino acid or derived from a natural alpha amino acid, and has the formula: 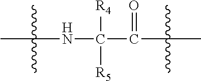
wherein R₄ is H; and
R₅ is the side chain, or second hydrogen of the amino acid
or a pharmaceutically acceptable salt or prodrug thereof.

50. A method or use according to any one of claims 1 to 31, wherein the compound has the structure of compound (1): 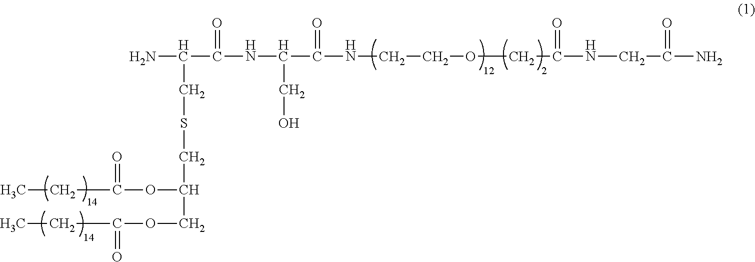
or a pharmaceutically acceptable salt or prodrug thereof.

51. A method or use according to any one of claims 1 to 31, wherein the compound has the structure of compound (5): 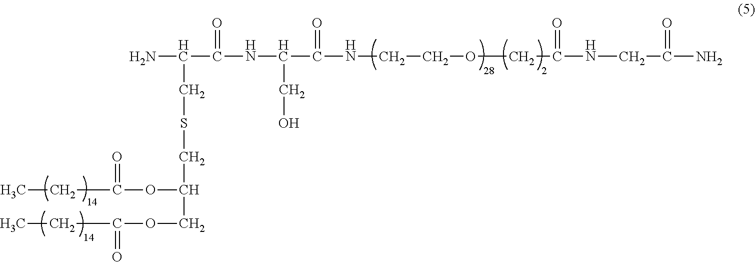
or a pharmaceutically acceptable salt or prodrug thereof.

52. A method or use according to any one of claims 1 to 31, wherein the compound is selected from the group consisting of:

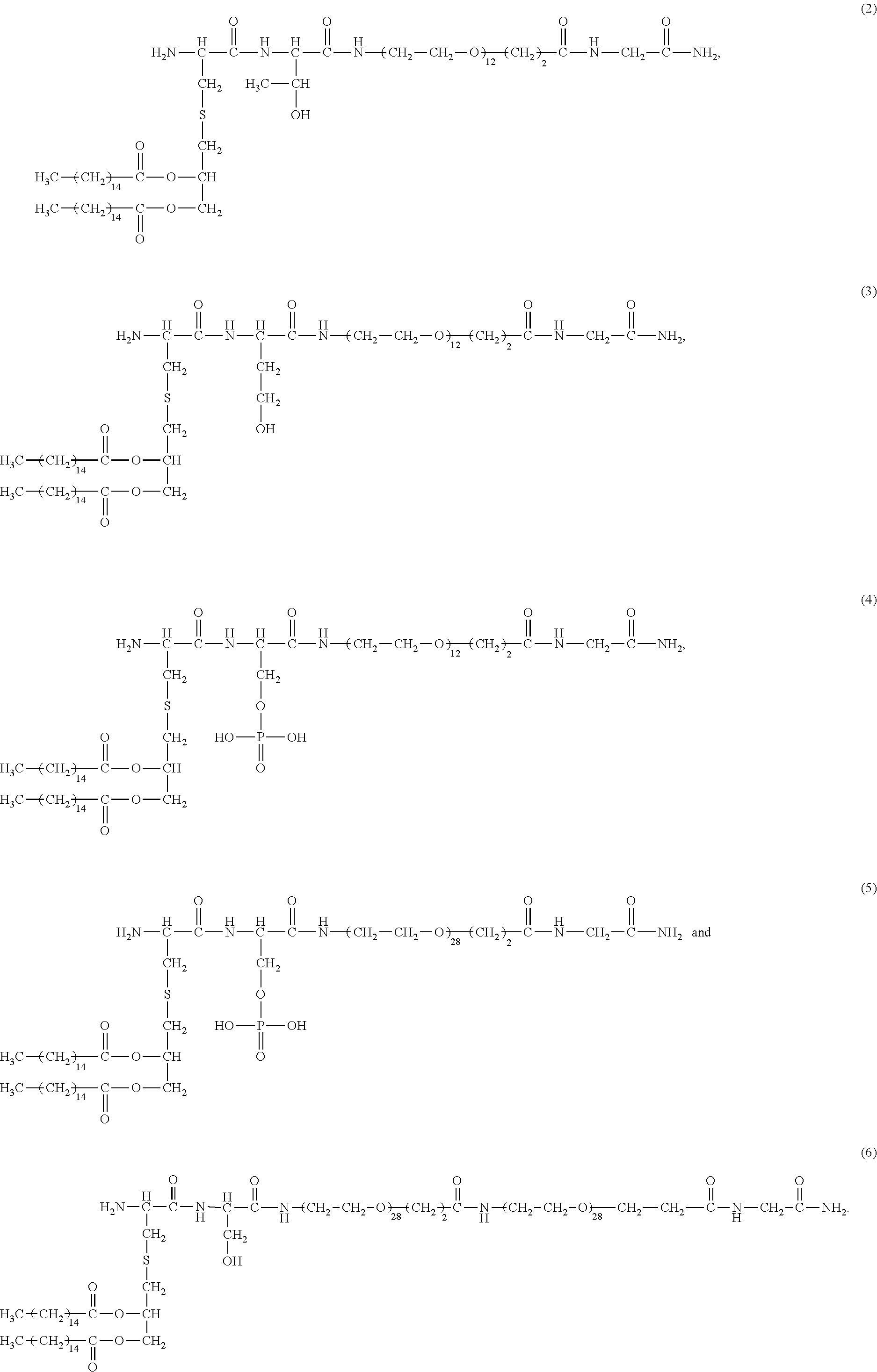 [SMILES: CCC(=O)OCC(CSCC(N)C(=O)NC(C(=O)NCCOCC(=O)NCC(N)=O)C(C)O)OC(=O)CC ; CCC(=O)OCC(CSCC(N)C(=O)NC(CCO)C(=O)NCCOCC(=O)NCC(N)=O)OC(=O)CC ; CCC(=O)OCC(CSCC(N)C(=O)NC(COP(=O)(O)O)C(=O)NCCOCC(=O)NCC(N)=O)OC(=O)CC ; CCC(=O)OCC(CSCC(N)C(=O)NC(COP(=O)(O)O)C(=O)NCCOCC(=O)NCC(N)=O)OC(=O)CC ; "and" ; CCC(=O)OCC(CSCC(N)C(=O)NC(CO)C(=O)NCCOCC(=O)NCCOCCC(=O)NCC(N)=O)OC(=O)CC]

53. A method or use according to any one of claims 1 to 31, wherein the compound comprising a TLR2 agonist comprises the structure:
A-Y-B
wherein A is: 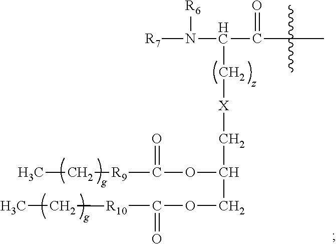
Y is 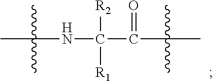
R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;
R₆ and R₇ are independently selected from the group consisting of H, a straight or branched C₁-C₄ alkyl, and —C(═O)CH₃;
R₈ is selected from the group consisting of H and a straight or branched C₁-C₆ alkyl;
R₉ and R₁₀ are independently selected from the group consisting of —NH—, —O— or a single bond;
each g is independently 10, 11, 12, 13, 14, 15, 16, 17 or 18;
z is 1 or 2;
X is S or S(═O);
and
B is polyethylene glycol (PEG),
or a pharmaceutically acceptable salt or prodrug thereof.

54. A method or use according to any one of claims 1 to 31, wherein the compound is of formula 00: 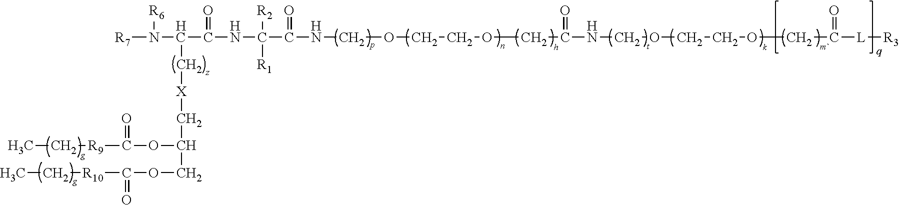
wherein
n is 3 to 100;
k is 3 to 100;
m is 1, 2, 3 or 4;
each g is independently 10, 11, 12, 13, 14, 15, 16, 17 or 18;
p is 2, 3 or 4;
t is 2, 3 or 4;
h is 1, 2, 3 or 4;
q is null or 1;
R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;
R₆ and R₇ are independently selected from the group consisting of H, a straight or branched C₁-C₄ alkyl, and —C(═O)CH₃;
R₈ is selected from the group consisting of H and a straight or branched C₁-C₆ alkyl;
R₉ and R₁₀ are independently selected from the group consisting of —NH—, —O— or a single bond;
z is 1 or 2;
X is S or S(═O);
wherein when q=1, R₃ is —NH₂ or —OH;
wherein when q=0, R₃ is H;
L is null or consists of 1 to 10 units, wherein each unit is a natural alpha amino acid or derived from a natural alpha amino acid, and has the formula: 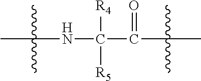
wherein R₄ is H; and
R₅ is the side chain, or second hydrogen of the amino acid,
or a pharmaceutically acceptable salt or prodrug thereof.

55. A method or use according to any one of claims 1 to 31, wherein the compound is:

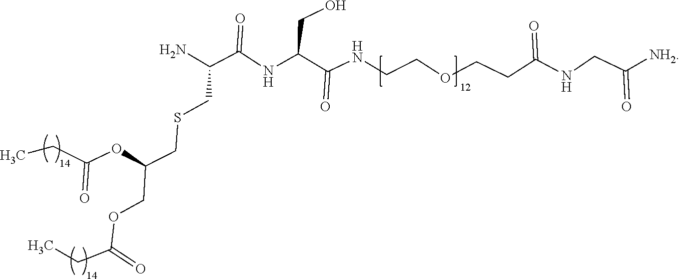

56. A method or use according to any one of claims 1 to 31, wherein the compound is:

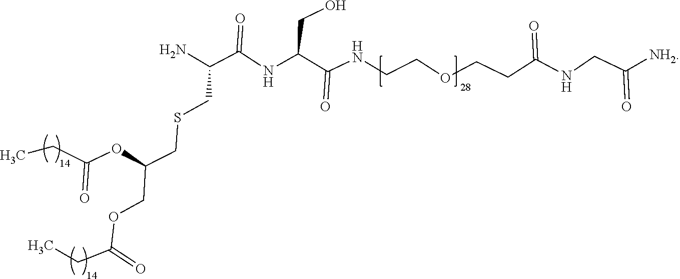

57. A method or use according to any one of claims 1 to 56, wherein the compound comprising a TLR2 agonist is administered to the individual in a single dose.

58. A method or use according to any one of claims 1 to 56, wherein the compound comprising a TLR2 agonist is administered to the individual in multiple doses.

59. A method or use according to any one of claims 1 to 58, wherein the TLR2 agonist is administered once per day.

60. A method or use according to any one of claims 1 to 58, wherein the TLR2 agonist is administered 3 times per day.

61. A method or use according to any one of claims 1 to 58, wherein the TLR2 agonist is administered once per week.

62. A method or use according to any one of claims 1 to 58, wherein the TLR2 agonist is administered three times per week.

63. A method or use according to any one of claims 1 to 62, wherein the TLR2 agonist is administered at a dose of between about 250 nmoles/kg body weight/dose to about 0.05 nmoles/kg body weight/dose.
